(19) United States
(12) Patent Application Publication
(10) Pub. No.: US 2023/0192805 A1
(43) Pub. Date: Jun. 22, 2023

(54) CHIMERIC RECEPTOR POLYPEPTIDES AND USES THEREOF

(71) Applicant: Nanjing Legend Biotech Co., Ltd., Nanjing, Jiangsu (CN)

(72) Inventors: Ming ZENG, Irving, TX (US); Huihui ZHANG, Nanjing, Jiangsu (CN)

(21) Appl. No.: 17/422,475

(22) PCT Filed: Jan. 14, 2020
(86) PCT No.: PCT/CN2020/071947

§ 371 Date: Jul. 13, 2021

(30) Foreign Application Priority Data
Jan. 14, 2019 (WO) PCT/CN2019/071609

(51) Int. Cl. C07K 14/725; C07K 16/28; A61K 35/17

(52) U.S. Cl. CPC C07K 14/7051; A61K 35/17; C07K 16/2809; C07K 16/2878; A61K 38/00; C07K 2317/622; C07K 2319/03; C07K 2319/33

(57) ABSTRACT

Provided is a chimeric receptor polypeptide comprising: a) an extracellular target binding domain; b) an extracellular TCR binding domain; c) a transmembrane domain comprising a transmembrane domain of a first TCR subunit; and/or d) an intracellular domain comprising an intracellular domain of a second TCR subunit. Also provided are nucleic acids encoding such chimeric receptor polypeptide and immune cells expressing such chimeric receptor polypeptide and uses thereof.

### CROSS REFERENCE TO RELATED APPLICATIONS

This application is a national stage application under 35 U.S.C. § 371 of International Patent Application No. PCT/CN2020/071947, filed on Jan. 14, 2020, which claims priority benefits of International Patent Application No. PCT/CN2019/071609, filed on Jan. 14, 2019, the contents of each of which are incorporated herein by reference in their entirety.

### SUBMISSION OF SEQUENCE LISTING ON ASCII TEXT FILE

The content of the following submission on ASCII text file is incorporated herein by reference in its entirely: a computer readable form (CRF) of the Sequence Listing (file name: 761422001500SEQLIST.TXT, date recorded: Jul. 7, 2021, size: 153 KB).

### FIELD OF THE INVENTION

This invention pertains to chimeric receptor polypeptide comprising an extracellular target binding domain, an extracellular T cell receptor (TCR) binding domain, a transmembrane domain, and/or an intracellular signaling domain.

### BACKGROUND OF THE INVENTION

The development and functions of all T cells depend on its antigen receptor. The T cell receptor (TCR) is a multi-protein complex, comprised of two functionally different modules: a ligand binding module and a signal transmission module. The ligand-binding module is composed of two variable polypeptide chains, TCRα and TCRβ, which form a covalently linked heterodimer and are responsible for the ligand specificity of the TCR. The signal-transmission module of the TCR complex is composed of invariant polypeptide chains, including CD3ε, CD3γ, CD3δ, and ζ. Among them, CD3ε, CD3γ, and CD3δ form non-covalently linked CD3εγ and CD3εδ heterodimers, whereas ζ forms a covalently linked ζζ homodimer. Surface expression of the TCR complex requires a fully assembled set of the complex subunits. Assembly begins with the formation, in the endoplasmic reticulum, of CD3εδ and CD3εγ heterodimers. These then associate with TCRα and TCRβ, respectively, to generate intermediate complexes. The ζζ homodimer is the last subunit to join, and upon its incorporation, the whole TCR complex is transported to the plasma membrane (Klausner et al., (1990); Exley et al., (1991); Dave et al., (1997); Marie-Cardine and Schraven, (1999); Kane et al., (2000); Matthew et al., (2004).

pMHC binding to TCRαβ is transmitted into the cell via the CD3-signaling units, involving TCR-CD3 clustering and conformational changes. Many experiments have shown that T cell activation involves a cascade of TCR-mediated signals that are regulated by three distinct intracellular signaling motifs located within the cytoplasmic tails of the CD3 chains (CD3 ζζ, CD3εγ, and CD3εδ) (Sun et al, J Immunol(185), (2010). Studies using chimeric molecules have demonstrated that the cytoplasmic tails of all signaling chains of the TCR complex can independently transduce signals leading to cellular cytotoxicity and/or cytokine production, bypassing the αβ recognition modality of the TCR. However, it was previously reported that signals through CD3ζ chain alone are insufficient to prime resting T lymphocytes (Thomas et al., J. Exp. Med., 1995), and mutated CD3ε signaling domain in mice showed incomplete T cell function (Matthew et al., J Immunol (193), 2014). Thus CD3εγ, CD3εδ, and ζζ chains complement each other in contributing to T cell functions, such as synergistic effect (Borroto et al., J Immunol (163), (1999).

Chimeric antigen receptor (CAR) is a modular fusion protein comprising binding domain, spacer domain, transmembrane domain, and intracellular signaling domain containing CD3ζ linked with one or two costimulatory molecules. CAR structure has evolved significantly from the initial composition involving only the CD3ζ signaling domain, dubbed a “first-generation CAR.” Since then, in an effort to augment T-cell persistence and proliferation, costimulatory end domains were added, giving rise to second- (e.g., CD3ζ plus 41BB- or CD28-signaling domains) and third-generation (e.g., CD3ζ plus 41BB- and CD28-signaling domains) CARs.

The adoptive transfer of CAR T cells has demonstrated remarkable success in treating blood-borne tumors; prominently, the use of CD 19 CARs in leukemias (Gill, S, et al, Blood Rev, (2015), and indications in patients with lymphoma and myeloma are being explored. A growing number of clinical trials have focused on solid tumors. Unfortunately, the clinical results have been much less encouraging. To date, the two most positive trials reported have used GD2 CARs to target neuroblastoma (3 of 11 patients with complete remissions) (Louis et al, Blood (118), (2011), and HER2 CARs for sarcoma (4 of 17 patients showing stable disease) (Ahmed et al, J Clin Oncol (33), (2015).

It has been suggested that poor trafficking, limited persistence and T-cell inhibitory activity in patients’ serum contributed to the observed lack of efficacy (Kershaw, et al. Clin. Cancer Res (12), (2006). Some armored CAR-T designs (constitutive secretion of immune-checkpoint inhibitors, IL-12, etc.) were expected to enhance antitumor efficacy by better T cell activity or better cell trafficking to the tumor (Oladapo et al, Nature scientific reports, (2017). While, new designs to improve the comprehensive functions of CAR T cells with better cell-killing effect, persistence in vivo and better tolerance to tumor microenvironments still core requirements.

T-cell mediated immunity is an adaptive process of developing antigen (Ag) - specific T lymphocytes to eliminate malignant cells. In recently years, fundamental advances in immunology and tumor biology have led to significant progress in the field of cell-based immunotherapy. For example, one promising approach has been developed in recent years to engage T cells for targeted cancer immunotherapy. This new approach is called Chimeric Antigen Receptor T cell Therapy (CAR-T). T cells equipped with CARs can be redirected to attack a broad variety of cells, including those that do not match the major histocompatibility complex (MHC) type of the T cell receptors (TCRs) on the T cells but express the target cell-surface antigens. Several attempts have also been made to engineer TCR molecules having antibody specificity. See, for example, WO2015/117229 and WO2016/187349.

One most notable adverse effect of current CAR-T and TCR-T approach is cytokine release syndrome (CRS) or cytokine storm. The basal signaling in the absence of a tumor antigen (tonic signaling) of current CAR-T and TCR-T can be substantial, which can increase differentiation and exhaustion of T cells and limit their potency. Due to tonic signaling, CAR-T and TCR-T cells exhibit cytokine production even in the absence of stimulation.

The disclosures of all publications, patents, patent applications and published patent applications referred to herein are hereby incorporated herein by reference in their entirety.

### BRIEF SUMMARY OF THE INVENTION

The present invention in one aspect provides a chimeric receptor polypeptide (also referred to herein as “STS polypeptides”) comprising: a) an extracellular target binding domain (e.g., anti-BCMA sdAb); b) an extracellular TCR binding domain (e.g., anti-CD3 scFv or sdAb, or anti-TCR scFv); c) a transmembrane domain; and d) optionally an intracellular domain, wherein the transmembrane domain and/or intracellular domain comprises a transmembrane and/or intracellular domain of a TCR subunit selected from the group consisting of CD3ε, CD3γ, CD3δ, TCRα, TCRβ, TCRγ, and TCRδ. In some embodiments, there is provided a chimeric receptor polypeptide comprising: a) an extracellular target binding domain (e.g., anti-BCMA sdAb); b) an extracellular TCR binding domain (e.g., anti-CD3 scFv or sdAb, or anti-TCR scFv); c) a transmembrane domain comprising a transmembrane domain of a first TCR subunit; and d) optionally an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ.

In some embodiments according to any of the embodiments described above, the extracellular TCR binding domain comprises a TCR antigen binding domain (e.g., sdAb, scFv) specifically recognizing a TCR subunit (e.g., extracellular domain of a TCR subunit) selected from the group consisting of TCRα, TCRβ, TCRγ, TCRδ, CD3ε, CD3δ, CD3γ, and CD3ζ, such as CD3ε or TCRγ/δ. In some embodiments, the TCR antigen binding domain is a single chain Fv (scFv; e.g., anti-CD3 scFv or anti-TCR scFv) or a single domain antibody (sdAb; e.g., anti-CD3 sdAb). In some embodiments, the extracellular TCR binding domain comprises two or more TCR antigen binding domains (e.g., scFv, sdAb), such as two or more TCR antigen binding domains arranged in tandem. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-TCR scFv comprising the amino acid sequence of SEQ ID NO: 27.

In some embodiments according to any of the embodiments described above, the first TCR subunit and the second TCR subunit are both selected from the group consisting of TCRα, TCRβ, TCRγ, and TCRδ, and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRγ, and TCRδ.

In some embodiments according to any of the embodiments described above, the chimeric receptor polypeptide does not comprise an extracellular domain of the first TCR subunit or the second TCR subunit. In some embodiments, the chimeric receptor polypeptide does not comprise an extracellular domain of any TCR subunit.

In some embodiments according to any of the embodiments described above, the chimeric receptor polypeptide does not comprise an intracellular co-stimulatory domain.

In some embodiments according to any of the embodiments described above, the first TCR subunit and the second TCR subunit are different. In some embodiments, the first TCR subunit is CD3ε. In some embodiments, the second TCR subunit is CD3ε.

In some embodiments according to any of the embodiments described above, the first TCR subunit and the second TCR subunit are the same. In some embodiments, the transmembrane domain of the chimeric receptor polypeptide comprises a transmembrane domain of CD3ε and the intracellular domain of the chimeric receptor polypeptide comprises an intracellular domain of CD3ε. In some embodiments, the transmembrane domain of the chimeric receptor polypeptide comprises a transmembrane domain of CD3γ and the intracellular domain of the chimeric receptor polypeptide comprises an intracellular domain of CD3γ. In some embodiments, the transmembrane domain of the chimeric receptor polypeptide comprises a transmembrane domain of CD3δ and the intracellular domain of the chimeric receptor polypeptide comprises an intracellular domain of CD3δ. In some embodiments, the transmembrane domain of the chimeric receptor polypeptide comprises a transmembrane domain of TCRα and the intracellular domain of the chimeric receptor polypeptide comprises an intracellular domain of TCRα. In some embodiments, the transmembrane domain of the chimeric receptor polypeptide comprises a transmembrane domain of TCRβ and the intracellular domain of the chimeric receptor polypeptide comprises an intracellular domain of TCRβ. In some embodiments, the transmembrane domain of the chimeric receptor polypeptide comprises a transmembrane domain of TCRγ and the intracellular domain of the chimeric receptor polypeptide comprises an intracellular domain of TCRγ. In some embodiments, the transmembrane domain of the chimeric receptor polypeptide comprises a transmembrane domain of TCRδ and the intracellular domain of the chimeric receptor polypeptide comprises an intracellular domain of TCRδ.

In some embodiments according to any of the embodiments described above, the extracellular target binding domain is N-terminal to the extracellular TCR binding domain. In some embodiments, the extracellular target binding domain is C-terminal to the extracellular TCR binding domain.

In some embodiments according to any of the embodiments described above, the extracellular target binding domain comprises a target antigen binding domains specifically recognizing a target antigen (e.g., BCMA). In some embodiments, the target antigen binding domain is an scFv, an sdAb (e.g., anti-BCMA sdAb), or a designed ankyrin repeat protein (DARPin). In some embodiments, the extracellular target binding domain comprises two or more target antigen binding domains (e.g., anti-BCMA sdAb), such as two or more target antigen binding domains arranged in tandem. In some embodiments, the two or more target antigen binding domains each specifically recognizes a same epitope on a same target antigen (e.g., same anti-BCMA sdAbs). In some embodiments, the two or more target antigen binding domains each specifically recognizes a different epitope on a same target antigen. In some embodiments, the two or more target antigen binding domains each specifically recognizes a different target antigen.

In some embodiments according to any of the embodiments described above, the target antigen is selected from the group consisting of BCMA, NY-ESO-1, VEGFR2, MAGE-A3, AFP, CD4, CD19, CD20, CD22, CD30, CD33, CD38, CD70, CD123, CEA, EGFR (such as EGFRvIII), GD2, GPC-2, GPC-3, HER2, LILRB4, IL-13Rα2, IGF1R, mesothelin, PSMA, ROR1, WT1, NKG2D, CLL1, Mesothelin, TGFaRII, TGFbRII, CCR5, CXCR4, CCR4, HPV related antigen, EBV related antigen (such as LMP1 and LMP2). In some embodiments, the target antigen is BCMA. In some embodiments, the target antigen binding domain is an anti-BCMA sdAb comprising the amino acid sequence of SEQ ID NO: 26.

In some embodiments according to any of the embodiments described above, the TCR subunit recognized by the TCR antigen binding domain, the first TCR subunit, and the second TCR subunit are all the same. In some embodiments, the TCR subunit recognized by the TCR antigen binding domain is different from the first TCR subunit or the second TCR subunit.

In some embodiments according to any of the embodiments described above, the chimeric receptor polypeptide further comprises a first linker connecting the extracellular target binding domain with the extracellular TCR binding domain. In some embodiments, the chimeric receptor polypeptide further comprise a second linker connecting the extracellular target binding domain and/or the extracellular TCR binding domain with the transmembrane domain. In some embodiments, the first linker and/or the second linker is a GS linker, an α-helical linker, a glycine-alanine polymer linker, an alanine-serine polymer linker, or an IgG4-Fc linker.

In some embodiments according to any of the embodiments described above, the chimeric receptor polypeptide further comprises a signal peptide at the N-terminus of the extracellular target binding domain or the extracellular TCR binding domain.

In some embodiments according to any of the embodiments described above, the chimeric receptor polypeptide further comprises a hinge region (e.g., CD8 hinge region) N-terminal to the transmembrane domain of the chimeric receptor polypeptide. In some embodiments, the hinge region comprises the amino acid sequence of SEQ ID NO: 31.

In some embodiments according to any of the embodiments described above, the chimeric receptor polypeptide comprises (or consists essentially of or consists of), from the N-terminus to the C-terminus: a) optional signal peptide - extracellular target binding domain -optional first linker - extracellular TCR binding domain - optional second linker - optional hinge region - transmembrane domain - intracellular domain; or b) optional signal peptide -extracellular TCR binding domain - optional first linker - extracellular target binding domain -optional second linker - optional hinge region - transmembrane domain - intracellular domain.

In some embodiments according to any of the embodiments described above, the chimeric receptor polypeptide comprises (or consists essentially of or consists of) from the N-terminus to the C-terminus: optional signal peptide - anti-BCMA sdAb - first linker - anti-CD3ε scFv - second linker - full length CD3ε without CD3ε signal peptide. In some embodiments, the chimeric receptor polypeptide comprises (or consists essentially of or consists of) from the N-terminus to the C-terminus: optional signal peptide - anti-BCMA sdAb - first linker - anti-CD3ε scFv - second linker - full length CD3γ without CD3γ signal peptide. In some embodiments, the chimeric receptor polypeptide comprises (or consists essentially of or consists of) from the N-terminus to the C-terminus: optional signal peptide - anti-BCMA sdAb - first linker - anti-CD3ε scFv - second linker - full length CD3δ without CD3δ signal peptide. In some embodiments, the chimeric receptor polypeptide comprises (or consists essentially of or consists of) from the N-terminus to the C-terminus: optional signal peptide - anti-BCMA sdAb - first linker - anti-CD3ε scFv - second linker - CD8 hinge region - CD3ε transmembrane domain - CD3ε intracellular domain. In some embodiments, the chimeric receptor polypeptide comprises (or consists essentially of or consists of) from the N-terminus to the C-terminus: optional signal peptide -anti-BCMA sdAb - first linker - anti-CD3ε scFv - second linker - CD8 hinge region - - CD3γ transmembrane domain - CD3γ intracellular domain. In some embodiments, the chimeric receptor polypeptide comprises (or consists essentially of or consists of) from the N-terminus to the C-terminus: optional signal peptide - anti-BCMA sdAb - first linker - anti-CD3ε scFv -second linker - CD8 hinge region - CD3δ transmembrane domain - CD3δ intracellular domain. In some embodiments, the chimeric receptor polypeptide comprises (or consists essentially of or consists of) from the N-terminus to the C-terminus: optional signal peptide - anti-BCMA sdAb -first linker - anti-CD3ε scFv - second linker - TCRα constant region - TCRα transmembrane domain - TCRα intracellular domain. In some embodiments, the chimeric receptor polypeptide comprises (or consists essentially of or consists of) from the N-terminus to the C-terminus: optional signal peptide - anti-BCMA sdAb - first linker - anti-CD3ε scFv - second linker -TCRβ constant region - TCRβ transmembrane domain - TCRβ intracellular domain. In some embodiments, the chimeric receptor polypeptide comprises (or consists essentially of or consists of) from the N-terminus to the C-terminus: optional signal peptide - anti-BCMA sdAb - first linker - anti-CD3ε scFv - second linker - TCRγ constant region - TCRγ transmembrane domain - TCRγ intracellular domain. In some embodiments, the chimeric receptor polypeptide comprises (or consists essentially of or consists of) from the N-terminus to the C-terminus: optional signal peptide - anti-BCMA sdAb - first linker - anti-CD3ε scFv - second linker -TCRδ constant region - TCRδ transmembrane domain - TCRδ intracellular domain. In some embodiments, the chimeric receptor polypeptide comprises (or consists essentially of or consists of) from the N-terminus to the C-terminus: optional signal peptide - anti-BCMA sdAb - first linker - anti-CD3ε scFv - second linker - TCRα transmembrane domain - TCRα intracellular domain. In some embodiments, the chimeric receptor polypeptide comprises (or consists essentially of or consists of) from the N-terminus to the C-terminus: optional signal peptide -anti-BCMA sdAb - first linker - anti-CD3ε scFv - second linker - TCRβ transmembrane domain - TCRβ intracellular domain. In some embodiments, the chimeric receptor polypeptide comprises (or consists essentially of or consists of) from the N-terminus to the C-terminus: optional signal peptide - anti-BCMA sdAb - first linker - anti-CD3ε scFv - second linker -TCRγ transmembrane domain - TCRγ intracellular domain. In some embodiments, the chimeric receptor polypeptide comprises (or consists essentially of or consists of) from the N-terminus to the C-terminus: optional signal peptide - anti-BCMA sdAb - first linker - anti-CD3ε scFv -second linker - TCRδ transmembrane domain - TCRδ intracellular domain. In some embodiments, the chimeric receptor polypeptide comprises (or consists essentially of or consists of) from the N-terminus to the C-terminus: optional signal peptide - anti-BCMA sdAb - first linker - anti-TCR scFv - second linker - TCRα constant region - TCRα transmembrane domain - TCRα intracellular domain. In some embodiments, the chimeric receptor polypeptide comprises (or consists essentially of or consists of) from the N-terminus to the C-terminus: optional signal peptide - anti-BCMA sdAb - first linker - anti-TCR scFv - second linker -TCRβ constant region - TCRβ transmembrane domain - TCRβ intracellular domain. In some embodiments, the chimeric receptor polypeptide comprises (or consists essentially of or consists of) from the N-terminus to the C-terminus: optional signal peptide - anti-BCMA sdAb - first linker - anti-TCR scFv - second linker - TCRγ constant region - TCRγ transmembrane domain - TCRγ intracellular domain. In some embodiments, the chimeric receptor polypeptide comprises (or consists essentially of or consists of) from the N-terminus to the C-terminus: optional signal peptide - anti-BCMA sdAb - first linker - anti-TCR scFv - second linker -TCRδ constant region - TCRδ transmembrane domain - TCRδ intracellular domain. In some embodiments, the chimeric receptor polypeptide comprises (or consists essentially of or consists of) from the N-terminus to the C-terminus: optional signal peptide - first anti-BCMA sdAb -first linker - second anti-BCMA sdAb - second linker - anti-CD3ε scFv - third linker - full length CD3ε without CD3ε signal peptide.

In some embodiments according to any of the embodiments described above, the first, second, and/or third linker comprises the sequence of any of SEQ ID NOs: 1-21, 67, and 68.

In another aspect, there is provided an isolated nucleic acid encoding any of the chimeric receptor polypeptides described above. Also provided are nucleic acid vectors comprising any of the chimeric receptor polypeptide-encoding nucleic acids described herein. In some embodiments, the nucleic acid vector comprises two or more any of the chimeric receptor polypeptide-encoding nucleic acids described herein connected via one or more linking sequences. In some embodiments, the linking sequence is selected from the group consisting of nucleic acids encoding P2A, T2A, E2A, F2A, BmCPV 2A, and BmIFV 2A, and internal ribosome entry site (IRES) sequence. In some embodiments, the nucleic acid vector comprises a nucleic acid encoding from the N-terminus to the C-terminus: optional first signal peptide - first anti-BCMA sdAb - first linker - first anti-CD3ε scFv - second linker - TCRα constant region -TCRα transmembrane domain - TCRα intracellular domain - P2A - optional second signal peptide - second anti-BCMA sdAb - third linker - second anti-CD3ε scFv - fourth linker -TCRβ constant region - TCRβ transmembrane domain - TCRβ intracellular domain. In some embodiments, the nucleic acid vector comprises a nucleic acid encoding from the N-terminus to the C-terminus: optional first signal peptide - first anti-BCMA sdAb - first linker - first anti-CD3ε scFv - second linker - TCRγ constant region - TCRγ transmembrane domain - TCRγ intracellular domain - P2A - optional second signal peptide - second anti-BCMA sdAb - third linker - second anti-CD3ε scFv - fourth linker - TCRδ constant region - TCRδ transmembrane domain - TCRδ intracellular domain. In some embodiments, the nucleic acid vector comprises a nucleic acid encoding from the N-terminus to the C-terminus: optional first signal peptide - first anti-BCMA sdAb - first linker - first anti-CD3ε scFv - second linker - TCRα transmembrane domain - TCRα intracellular domain - P2A - optional second signal peptide - second anti-BCMA sdAb - third linker - second anti-CD3ε scFv - fourth linker - TCRβ transmembrane domain - TCRβ intracellular domain. In some embodiments, the nucleic acid vector comprises a nucleic acid encoding from the N-terminus to the C-terminus: optional first signal peptide - first anti-BCMA sdAb - first linker - first anti-CD3ε scFv - second linker - TCRγ transmembrane domain - TCRγ intracellular domain - P2A - optional second signal peptide - second anti-BCMA sdAb - third linker - second anti-CD3ε scFv - fourth linker - TCRδ transmembrane domain - TCRδ intracellular domain. In some embodiments, the nucleic acid vector comprises a nucleic acid encoding from the N-terminus to the C-terminus: optional first signal peptide - first anti-BCMA sdAb - first linker - first anti-TCR scFv - second linker - TCRγ constant region -TCRγ transmembrane domain - TCRγ intracellular domain - P2A - optional second signal peptide - second anti-BCMA sdAb - third linker - second anti-TCR scFv - fourth linker - TCRδ constant region - TCRδ transmembrane domain - TCRδ intracellular domain.

In another aspect, there is provided an isolated immune cell comprising one or more chimeric receptor polypeptides described above, one or more nucleic acids described above, or one or more nucleic acid vectors described above. In some embodiments, the isolated immune cell comprises two or more chimeric receptor polypeptides described above. In some embodiments, there is provided an isolated immune cell comprising any of the nucleic acids or nucleic acid vectors described above. In some embodiments, the isolated immune cell is selected from the group consisting of Tαβ cells, Tγδ cells, effector T cells, memory T cells, cytotoxic T cells, T helper cells, Natural Killer T (NKT) cells, regulatory T cells (Tregs), tumor infiltrating lymphocytes (TILs). In some embodiments, the isolated immune cell is a T cell (e.g., effector T cell). In some embodiments, the isolated immune cell further comprises a chimeric antigen receptor (CAR) and/or an engineered TCR.

In another aspect, there is provided a pharmaceutical composition comprising any of the immune cells described above, and an optional pharmaceutically acceptable excipient. In some embodiments, there is provided a method of treating a disease in an individual (e.g., human), comprising administering to the individual an effective amount of any of the immune cells described above, or any of the pharmaceutical compositions described above. In some embodiments, the pharmaceutical composition is administered intravenously, intratumorally, or subcutaneously. In some embodiments, the disease is cancer. In some embodiments, the cancer is selected from the group consisting of acute leukemias (including but not limited to acute myeloid leukemia (AML), B-cell acute lymphoid leukemia (BALL), T-cell acute lymphoid leukemia (TALL), and acute lymphoid leukemia (ALL)), chronic leukemias (including but not limited to chronic myelogenous leukemia (CML) and chronic lymphocytic leukemia (CLL)), multiple myeloma (MM), myelodysplastic syndrome (MDS), myeloproliferative neoplasms (MPNs), chronic myeloid leukemia (CML), and blastic plasmacytoid dendritic cell neoplasm (BPDCN).

Also provided are methods of making any of the constructs described herein, articles of manufacture, and kits that are suitable for the methods described herein.

### BRIEF DESCRIPTION OF THE DRAWINGS

FIG. 1 shows an exemplary configuration of a chimeric receptor polypeptide expressed on the surface of a T cell, comprising a target antigen binding domain, a TCR antigen binding domain, and CD3ε. As illustrated in FIG. 1, without being bound by the theory, the chimeric receptor polypeptide may transduce TCR signaling via another TCR complex.

FIG. 2 shows an exemplary configuration of a chimeric receptor polypeptide expressed on the surface of a T cell, comprising a target antigen binding domain, a TCR antigen binding domain, and CD3γ. As illustrated in FIG. 2, without being bound by the theory, the chimeric receptor polypeptide may transduce TCR signaling via another TCR complex.

FIG. 3 shows an exemplary configuration of a chimeric receptor polypeptide expressed on the surface of a T cell, comprising a target antigen binding domain, a TCR antigen binding domain, and CD3δ. As illustrated in FIG. 3, without being bound by the theory, the chimeric receptor polypeptide may transduce TCR signaling via another TCR complex.

FIG. 4 shows an exemplary configuration of a chimeric receptor polypeptide expressed on the surface of a T cell, comprising a target antigen binding domain, a TCR antigen binding domain, and TCRα. As illustrated in FIG. 4, without being bound by the theory, the chimeric receptor polypeptide may transduce TCR signaling via another TCR complex.

FIG. 5 shows an exemplary configuration of a chimeric receptor polypeptide expressed on the surface of a T cell, comprising a target antigen binding domain, a TCR antigen binding domain, and TCRβ. As illustrated in FIG. 5, without being bound by the theory, the chimeric receptor polypeptide may transduce TCR signaling via another TCR complex.

FIG. 6 shows an exemplary configuration of a chimeric receptor polypeptide expressed on the surface of a T cell, comprising a target antigen binding domain, a TCR antigen binding domain, and CD3ε. As illustrated in FIG. 6, without being bound by the theory, the chimeric receptor polypeptide may transduce TCR signaling in the same TCR complex into which the chimeric receptor polypeptide is incorporated.

FIG. 7 shows an exemplary configuration of a chimeric receptor polypeptide expressed on the surface of a T cell, comprising a target antigen binding domain, a TCR antigen binding domain, and the transmembrane and intracellular domains of a TCR subunit (including CD3ε, CD3γ, CD3δ, TCRα, TCRβ, TCRγ, and TCRδ), without an extracellular domain of the TCR subunit. As illustrated in FIG. 7, without being bound by the theory, two chimeric receptor polypeptides can dimerize with each other and function together to transduce TCR signaling, and the antigen binding domain on the two chimeric receptor polypeptides can be the same or different.

FIG. 8 shows an exemplary configuration of a chimeric receptor polypeptide expressed on the surface of a T cell, comprising a target antigen binding domain, an extracellular TCR binding domain comprising two TCR antigen binding domains arranged in tandem, and the transmembrane and intracellular domain of a TCR subunit (including CD3ε, CD3γ, CD3δ, TCRα, TCRβ, TCRγ, and TCRδ), without an extracellular domain of the TCR subunit. As illustrated in FIG. 8, without being bound by the theory, two chimeric receptor polypeptides can dimerize with each other and function together to transduce TCR signaling, the antigen binding domains on the two chimeric receptor polypeptides can be the same or different, and the TCR antigen binding domains on the two chimeric receptor polypeptides can be the same or different.

FIG. 9 shows an exemplary configuration of a chimeric receptor polypeptide expressed on the surface of a T cell, comprising an extracellular target binding domain comprising two target antigen binding domains (which can be the same or different) arranged in tandem, a TCR antigen binding domain, and the transmembrane and intracellular domains of a TCR subunit (including CD3ε, CD3γ, CD3δ, TCRα, TCRβ, TCRγ, and TCRδ), without an extracellular domain of the TCR subunit. As illustrated in FIG. 9, without being bound by the theory, two chimeric receptor polypeptides can dimerize with each other and function together to transduce TCR signaling, and target antigen binding domains on the two chimeric receptor polypeptides can be the same or different, and the TCR antigen binding domains on the two chimeric receptor polypeptides can be the same or different.

FIGS. 10A-10B provide cell killing assay result (FIG. 10A) and IFNy secretion result (FIG. 10B) of various STS-T cells (T cells expressing the STS polypeptides) on RPMI8226 cells, including STS-T cells expressing sdAbBCMA-anti-CD3 scFv-e, sdAbBCMA-anti-CD3 scFv-g, and sdAb BCMA-anti CD3 scFv-d. Untransfected T cells (UnT) served as control.

FIGS. 11A-11B provide cell killing assay result (FIG. 11A) and IFNy secretion result (FIG. 11B) of various STS-T cells on RPMI8226 cells, including STS-T cells expressing sdAbBCMA-anti-CD3 scFv-se, sdAbBCMA-anti-CD3 scFv-sg, and sdAbBCMA-anti-CD3 scFv-sd. Untransfected T cells (UnT) served as control.

FIG. 12 shows the expression of exogenous receptor, endogenous TCR, and endogenous CD3 on untransfected T cells (UnT), T cells transfected with an sdAbBCMA-anti-CD3 scFv-sd construct.

FIG. 13 shows cell killing assay results of STS-T cells expressing sdAbBCMA-anti-TCR Ab-tgC/tdC on H929/Luc cells. Untransfected T cells (UnT) served as control.

FIG. 14 pro shows vide cell killing assay results of STS-T cells expressing sdAbBCMA-anti-CD3 scFv-sta/stb on CHO/BCMA cells. Untransfected T cells (UnT) served as control.

FIG. 15 shows cell killing assay results of STS-T cells expressing tandem sdAbBCMA-anti-CD3 scFv-e on CHO/BCMA cells. Untransfected T cells (UnT) served as control.

FIG. 16A shows a vector construct encoding anti-BCMA-anti-CD3 scFv-CD3ε. FIG. 16B shows a vector construct of encoding anti-BCMA-anti-CD3 scFv -se. FIG. 16C shows a vector construct encoding BCMA-anti-TCR-tgC/tdC. FIG. 16D shows a vector construct encoding anti-BCMA-anti-CD3 scFv-sta/stb.

### DETAILED DESCRIPTION OF THE INVENTION

The present invention provides a chimeric receptor polypeptide (also referred to herein as “STS polypeptides”) comprising: a) an extracellular target binding domain; b) an extracellular TCR binding domain; c) a transmembrane domain; and d) an intracellular domain, wherein the transmembrane domain and/or the intracellular domain comprises a transmembrane and/or an intracellular domain of a TCR subunit selected from the group consisting of CD3ε, CD3γ, CD3δ, TCRα, TCRβ, TCRγ, and TCRδ. Also provided are isolated nucleic acids and vectors encoding STS polypeptides, engineered immune cells (e.g., T cells) expressing STS polypeptides, pharmaceutical compositions thereof, and use of such STS polypeptides or pharmaceutical compositions thereof for treating diseases (such as cancer).

The STS polypeptides, when expressed in an immune cell, e.g., a T cell such as effector T cell, showed significant cell killing effect. Surprisingly, the cell killing effect of the engineered T cells expressing STS polypeptides (referred to as “STS-T cell”) did not require the presence of an intracellular co-stimulatory domain in the STS polypeptide, even though the intracellular domains of CD3ε, CD3γ, CD3δ, TCRα, TCRβ, TCRγ, and TCRδ are traditionally believed to either lack a signaling domain or be insufficient to stimulate a T cell without co-stimulation. Further, cell killing effect was observed even when the STS polypeptide does not contain any extracellular domain of a TCR subunit, which is believed to be required for transmitting an extracellular signal to the intracellular domain(s). Comparing to prior constructs such as chimeric antigen receptor (CAR) armed T cells (CAR-T), the STS-T cells had low cytokine release upon antigen stimulation, had improved ex vivo expansion properties, and persisted longer in vivo upon administration. Further, unlike engineered T cell receptor (engineered TCR) and T cell Antigen Coupler (TAC) platforms (e.g., engineered TCR-T cells, TAC-T cells), endogenous TCR and CD3 molecules on STS-T cells were not down-regulated on the cell surface as one would have expected in a cell expressing a binding domain for a TCR subunit (e.g., anti-CD3ε scFv or sdAb, or anti-TCR scFv).

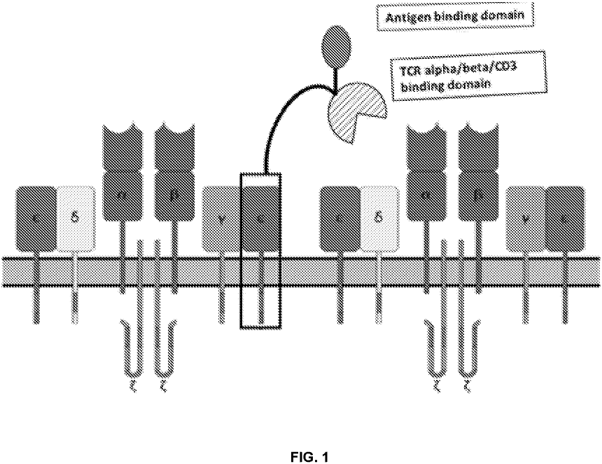

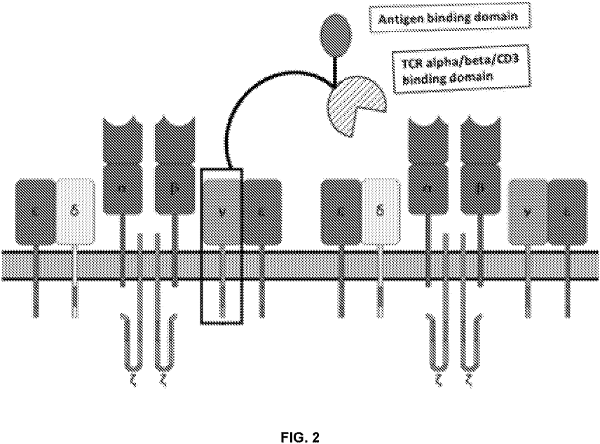

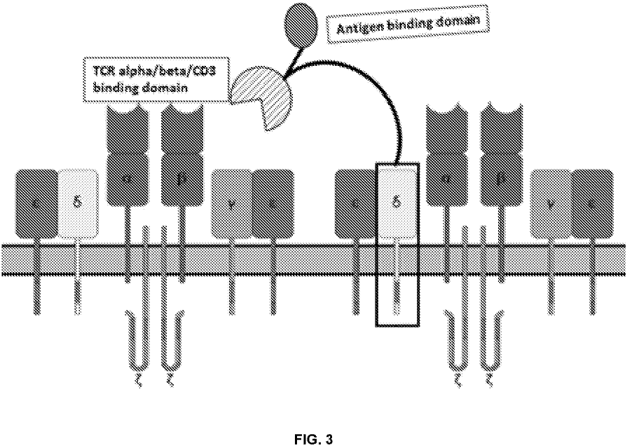

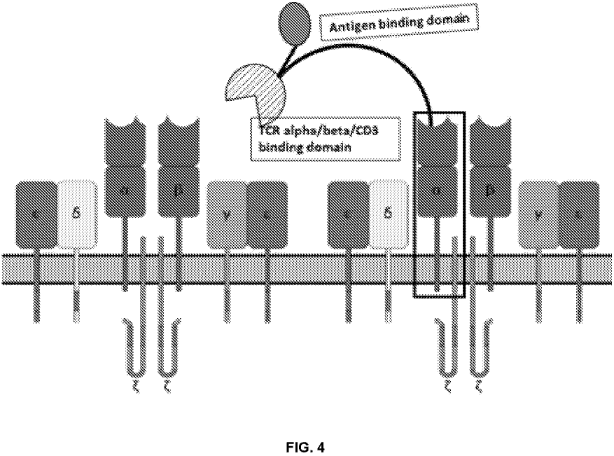

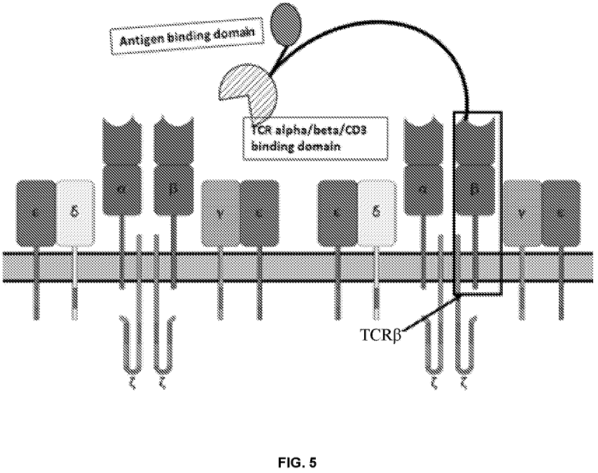

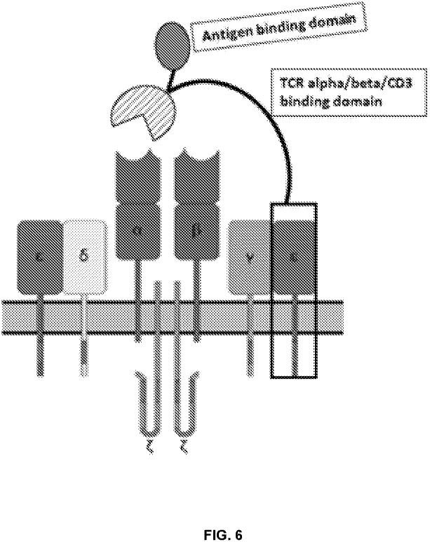

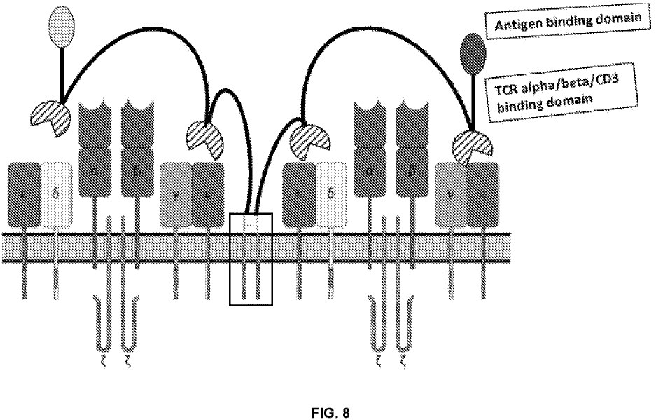

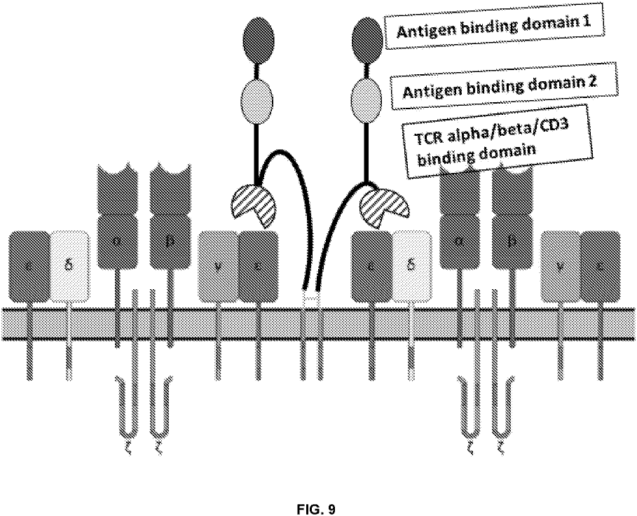

Without being bound by theory, the extracellular target binding domain of the STS polypeptide is able to specifically bind to a target antigen (e.g., BCMA) and bring the immune cell (e.g., T cell) expressing the STS polypeptides to the proximity of the target cell (e.g., tumor cell). The extracellular TCR binding domain is able to bind to a TCR subunit (e.g., extracellular domain of a TCR subunit) in a TCR complex on the surface of the immune cell (e.g., T cell) and transmits signal through the endogenous TCR complex. Additionally or alternatively, the STS polypeptide may be incorporated into a TCR complex together with one or more endogenous TCR subunits, and transduces the signal in the same TCR complex. The various configurations and possible mechanisms of action of an STS polypeptide are shown in FIGS. 1-9.

Thus, the present application in one aspect provides a chimeric receptor polypeptide comprising an extracellular target binding domain, an extracellular TCR binding domain, a transmembrane, and an intracellular domain, wherein the transmembrane domain and/or intracellular domain comprises the transmembrane domain and/or intracellular domain of a TCR subunit selected from the group consisting of CD3ε, CD3γ, CD3δ, TCRα, TCRβ, TCRγ, and TCRδ. Also provided are nucleic acids encoding such chimeric receptor polypeptide, and nucleic acid vectors comprising such nucleic acids

In another aspect, there is provided an immune cell expressing on its surface a chimeric receptor polypeptide comprising an extracellular target binding domain, an extracellular TCR binding domain, a transmembrane, and an intracellular domain, wherein the transmembrane domain and/or intracellular domain comprises the transmembrane domain and/or intracellular domain of a TCR subunit selected from the group consisting of CD3ε, CD3γ, CD3δ, TCRα, TCRβ, TCRγ, and TCRδ.

Also provided are methods of making and using immune cells expressing a chimeric receptor polypeptide for treatment purposes, as well as kits and articles of manufacture useful for such methods.

### Definitions

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” for cancer if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” is at least the minimum amount required to effect a measurable improvement of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder.

As used herein, “in conjunction with” refers to administration of one treatment modality in addition to another treatment modality. As such, “in conjunction with” refers to administration of one treatment modality before, during, or after administration of the other treatment modality to the individual.

A “subject” or an “individual” for purposes of treatment refers to any animal classified as a mammal, including humans, domestic and farm animals, and zoo, sports, or pet animals, such as dogs, horses, cats, cows, etc.

The term “antibody” herein is used in the broadest sense and specifically covers monoclonal antibodies (including full length monoclonal antibodies), multispecific antibodies (e.g., bispecific antibodies), and antibody fragments so long as they exhibit the desired biological activity.

The terms “native antibody”, “full length antibody,” “intact antibody” and “whole antibody” are used herein interchangeably to refer to an antibody in its substantially intact form, not antibody fragments as defined below. The terms particularly refer to an antibody with heavy chains that contain an Fc region. Native antibodies are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the C_{H}1, C_{H}2 and C_{H}3 domains (collectively, C_{H}) of the heavy chain and the C_{L} domain of the light chain.

The “variable region” or “variable domain” of an antibody refers to the amino-terminal domains of the heavy or light chain of the antibody. The variable domain of the heavy chain may be referred to as “V_{H}.” The variable domain of the light chain may be referred to as “V_{L}.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various immune effector functions, such as participation of the antibody in antibody-dependent cellular toxicity (ADCC).

The “light chains” of antibodies (immunoglobulins) from any mammalian species can be assigned to one of two clearly distinct types, called kappa (“κ”) and lambda (“λ”), based on the amino acid sequences of their constant domains

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and µ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody, preferably comprising the antigen binding region thereof. In some embodiments, the antibody fragment described herein is an antigen-binding fragment. Examples of antibody fragments include Fab, Fab′, F(ab′)₂, and Fv fragments; diabodies; linear antibodies; single-chain antibody molecules, such as single-chain variable fragment (scFv); single domain antibodies (sdAbs), such as V_{H}H fragments or V_{NAR} fragments; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, The Pharmacology of Monoclonal Antibodies. Springer Berlin Heidelberg, 1994. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9: 129-134 (2003).

The term “heavy chain-only antibody” or “HCAb” refers to a functional antibody, which comprises heavy chains, but lacks the light chains usually found in antibodies. Camelid animals (such as camels, llamas, or alpacas) are known to produce HCAbs.

The term “single-domain antibody” or “sdAb” refers to an antibody fragment consisting of a single monomeric variable antibody domain. In some cases, single domain antibodies are engineered from camelid HCAbs, and such sdAbs are referred herein as “nanobodies” or “V_{H}Hs”. Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual, (Cold Spring Harbor Laboratory Press, 2nd ed. 1988); Hammerling et al., Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits el al., Nature 362: 255-258 (1993); Bruggemann et αl., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg el al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995)).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison etal., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol. 227:381 (1991); Marks et al., J. Mol. Biol. 222:581 (1991). Also available for the preparation of human monoclonal antibodies are methods described in Cole et al., Monoclonal Antibodies and Cancer Therapy, Alan R. Liss, 77 (1985); Boerner et al., J. Immunol. 147(1):86-95 (1991). See also van Dijk and van de Winkel, Curr. Opin Pharmacol. 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSETM technology). See also, for example, Li et al., Proc. Natl. Acad. Sci. USA 103:3557-3562 (2006) regarding human antibodies generated via a human B-cell hybridoma technology.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤ 1 µM, ≤ 100 nM, ≤ 10 nM, ≤ 1 nM, or ≤ 0.1 nM. In certain embodiments, an antibody specifically binds to an epitope on a protein that is conserved among the protein from different species. In another embodiment, specific binding can include, but does not require exclusive binding.

“Chimeric antigen receptor” or “CAR” as used herein refers to genetically engineered receptors, which graft one or more antigen specificity onto cells, such as T cells. CARs are also known as “artificial T-cell receptors,” “chimeric T cell receptors,” or “chimeric immune receptors.” In some embodiments, the CAR comprises an extracellular variable domain of an antibody specific for a tumor antigen, and an intracellular signaling domain of a T cell or other receptors, such as one or more co-stimulatory signaling domains. “CAR-T” refers to a T cell that expresses a CAR.

“T cell receptor” or “TCR” as used herein refers to endogenous or recombinant/engineered T cell receptor comprising an extracellular antigen binding domain that binds to a specific antigenic peptide bound in an MHC molecule. In some embodiments, the TCR comprises a TCRα polypeptide chain and a TCR β polypeptide chain. In some embodiments, the TCR comprises a TCRγ polypeptide chain and a TCRδ polypeptide chain. In some embodiments, the TCR specifically binds a tumor antigen. In some embodiments, the TCR specifically binds a tumor antigen/MHC. “TCR-T” refers to a T cell that expresses a recombinant/engineered TCR.

“TCR complex” as used herein refers to a complex of TCR and CD3. “TCR subunits” used herein refers to a subunit of the TCR complex, which include, for example, TCRα, TCRβ, TCRγ, TCRδ, CD3ε, CD3γ, CD3δ, and CD3ζ.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

The term “express” refers to translation of a nucleic acid into a protein. Proteins may be expressed and remain intracellular, become a component of the cell surface membrane, or be secreted into extracellular matrix or medium.

The term “host cell” refers to a cell which can support the replication or expression of the expression vector. Host cells may be prokaryotic cells such as E.coli, or eukaryotic cells, such as yeast, insect cells, amphibian cells, or mammalian cells (e.g., CHO cells, immune cells such as effector T cells).

The term “transfected” or “transformed” or “transduced” as used herein refers to a process by which exogenous nucleic acid is transferred or introduced into the host cell A “transfected” or “transformed” or “transduced” cell is one which has been transfected, transformed or transduced with exogenous nucleic acid.

The term “in vivo” refers to inside the body of the organism from which the cell is obtained. “Ex vivo” or “in vitro” means outside the body of the organism from which the cell is obtained.

The term “cell” includes the primary subject cell and its progeny.

As used herein, the term “immunomodulator” refers to any protein or peptide-based agent that has an effect (such as inhibitory or stimulatory effect) on the immune system.

As used herein, the term “immune checkpoint inhibitor” refers to a molecule that totally or partially reduces, inhibits or interferes with one or more checkpoint proteins, which can regulate T-cell activation and function.

As used herein, the term “immunoactivator” refers to a molecule that stimulates, activates, or increases the intensity of an immune response.

As used herein, the term “therapeutic protein” refers to any protein or peptide-based agent that has a therapeutic effect.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### Chimeric Receptor Polypeptides (STS Polypeptides)

In one aspect, the present invention provides a chimeric receptor polypeptide (or STS polypeptide). In some embodiments, there is provided a chimeric receptor polypeptide comprising (or consisting essentially of or consisting of): a) an extracellular target binding domain (e.g., anti-BCMA sdAb); b) an extracellular TCR binding domain (e.g., anti-CD3 scFv or sdAb, or anti-TCR scFv); c) a transmembrane domain, and d) an intracellular domain comprising an intracellular domain of a TCR subunit, wherein the TCR subunit is selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of): a) an extracellular target binding domain (e.g., anti-BCMA sdAb); b) an extracellular TCR binding domain (e.g., anti-CD3 scFv or sdAb, or anti-TCR scFv); and c) a transmembrane domain comprising a transmembrane domain of a TCR subunit, wherein the TCR subunit is selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of): a) an extracellular target binding domain (e.g., anti-BCMA sdAb); b) an extracellular TCR binding domain (e.g., anti-CD3 scFv or sdAb, or anti-TCR scFv); c) a transmembrane domain comprising a transmembrane domain of a TCR subunit, wherein the TCR subunit is selected from the group consisting of CD3ε, CD3γ, CD3δ, TCRα, TCRβ, TCRγ, and TCRδ, and d) an intracellular domain. In some embodiments, the chimeric receptor polypeptide does not comprise an extracellular domain of the TCR subunit (or an extracellular domain of any TCR subunit). In some embodiments, the TCR subunit is selected from the group consisting of TCRα, TCRβ, TCRγ, and TCRδ and the chimeric receptor polypeptide does not comprise a variable region of the extracellular domain of the TCR subunit (or a variable region of the extracellular domain of any of TCRα, TCRβ, TCRγ, and TCRδ. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain.

In some embodiments, there is provided a chimeric receptor polypeptide comprising (or consisting essentially of or consisting of): a) an extracellular target binding domain (e.g., anti-BCMA sdAb); b) an extracellular TCR binding domain (e.g., anti-CD3 scFv or sdAb, or anti-TCR scFv); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the first TCR subunit is CD3ε and/or the second TCR subunit is CD3ε. In some embodiments, the first TCR subunit is CD3γ and/or the second TCR subunit is CD3γ. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRγand/or the second TCR subunit is TCRγ In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are different. In some embodiments, the first TCR subunit and the second TCR subunit are both selected from the group consisting of TCRα, TCRβ, TCRy, and TCRδ and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an extracellular domain of the first TCR subunit and/or the second TCR subunit. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain.

In some embodiments, there is provided a chimeric receptor polypeptide comprising (or consisting essentially of or consisting of), from the N-terminus to the C-terminus: a) an optional signal peptide; b) an extracellular target binding domain (e.g., anti-BCMA sdAb); c) an optional first linker (e.g., GS linker), d) an extracellular TCR binding domain (e.g., anti-CD3 scFv or sdAb, or anti-TCR scFv); e) an optional second linker (e.g., GS linker); f) an optional hinge region (e.g., CD8 hinge region); g) a transmembrane domain comprising a transmembrane domain of a first TCR subunit; and h) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of), from the N-terminus to the C-terminus: a) an optional signal peptide; b) an extracellular TCR binding domain (e.g., anti-CD3 scFv or sdAb, or anti-TCR scFv); c) an optional first linker (e.g., GS linker), d) an extracellular target binding domain (e.g., anti-BCMA sdAb); e) an optional second linker (e.g., GS linker); f) an optional hinge region (e.g., CD8 hinge region); g) a transmembrane domain comprising a transmembrane domain of a first TCR subunit; and h) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the first TCR subunit is CD3ε and/or the second TCR subunit is CD3ε. In some embodiments, the first TCR subunit is CD3γ and/or the second TCR subunit is CD3γ. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRy and/or the second TCR subunit is TCRy. In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are different. In some embodiments, the first TCR subunit and the second TCR subunit are both selected from the group consisting of TCRα, TCRβ, TCRy, and TCRδ and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an extracellular domain of the first TCR subunit and/or the second TCR subunit. In some embodiments, the chimeric receptor polypeptide does not comprise an extracellular domain of any TCR subunits. In some embodiments, the chimeric receptor polypeptide does not comprise an intracellular co-stimulatory domain.

In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to a TCR subunit (e.g., extracellular domain of a TCR subunit, such as extracellular domain of CD3ε or TCR); c) a transmembrane domain, and d) an intracellular domain comprising an intracellular domain of a TCR subunit, wherein the TCR subunit is selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to a TCR subunit (e.g., extracellular domain of a TCR subunit, such as extracellular domain of CD3ε or TCR); and c) a transmembrane domain comprising a transmembrane domain of a TCR subunit, wherein the TCR subunit is selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to a TCR subunit (e.g., extracellular domain of a TCR subunit, such as extracellular domain of CD3ε or TCR); c) a transmembrane domain comprising a transmembrane domain of a TCR subunit, wherein the TCR subunit is selected from the group consisting of CD3ε, CD3y, CD3δ, TCRα, TCRβ, TCRy, and TCRδ, and d) an intracellular domain. In some embodiments, the TCR subunit is selected from the group consisting of TCRα, TCRβ, TCRy, and TCRδ and the chimeric receptor polypeptide does not comprise a variable region of the extracellular domain of the TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an extracellular domain of the TCR subunit (or an extracellular domain of any TCR subunit). In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the target antigen binding domain is an sdAb specifically binding to BCMA, such as any of the anti-BCMA sdAbs disclosed in PCT/CN2016/094408 and PCT/CN2017/096938, the contents of which are incorporated herein by reference in their entirety. In some embodiments, the anti-BCMA sdAb comprises the amino acid sequence of SEQ ID NO: 26. In some embodiments, the TCR antigen binding domain (e.g., scFv, sdAb) specifically binds to CD3ε(e.g., N-terminus of CD3ε. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-TCR scFv comprising the amino acid sequence of SEQ ID NO: 27. In some embodiments, the extracellular TCR binding domain comprises two or more TCR antigen binding domains (e.g., scFv or sdAb) arranged in tandem. In some embodiments, the extracellular target binding domain comprises two or more target antigen binding domains (e.g., scFv, sdAb, or DARPin) arranged in tandem. In some embodiments, the chimeric receptor polypeptide further comprises a hinge region (e.g., CD8 hinge region) N-terminal to the transmembrane domain of the chimeric receptor polypeptide. In some embodiments, the chimeric receptor polypeptide further comprises a first linker connecting the extracellular target binding domain with the extracellular TCR binding domain. In some embodiments, the chimeric receptor polypeptide further comprises a second linker connecting the extracellular target binding domain and/or the extracellular TCR binding domain with the transmembrane domain. In some embodiments, the first linker and/or the second linker is a GS linker, an α-helical linker, a glycine-alanine polymer linker, an alanine-serine polymer linker, or an IgG4-Fc linker. In some embodiments, the first linker and/or the second linker comprises the amino acid sequence of any of SEQ ID NOs: 1-21, 67, and 68.

In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to a TCR subunit (e.g., extracellular domain of a TCR subunit, such as extracellular domain of CD3ε or TCR); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the first TCR subunit is CD3ε and/or the second TCR subunit is CD3ε. In some embodiments, the first TCR subunit is CD3γ and/or the second TCR subunit is CD3γ. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRy and/or the second TCR subunit is TCRy. In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are different. In some embodiments, the first TCR subunit and the second TCR subunit are both selected from the group consisting of TCRα, TCRβ, TCRy, and TCRδ and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an extracellular domain of the first and/or second TCR subunits. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the target antigen binding domain is an sdAb specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the TCR antigen binding domain (e.g., scFv, sdAb) specifically binds to CD3ε(e.g., N-terminus of CD3ε). In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-TCR scFv comprising the amino acid sequence of SEQ ID NO: 27. In some embodiments, the extracellular TCR binding domain comprises two or more TCR antigen binding domains (e.g., scFv or sdAb) arranged in tandem. In some embodiments, the extracellular target binding domain comprises two or more target antigen binding domains (e.g., scFv, sdAb, or DARPin) arranged in tandem. In some embodiments, the chimeric receptor polypeptide further comprises a hinge region (e.g., CD8 hinge region) N-terminal to the transmembrane domain of the chimeric receptor polypeptide. In some embodiments, the chimeric receptor polypeptide further comprises a first linker connecting the extracellular target binding domain with the extracellular TCR binding domain. In some embodiments, the chimeric receptor polypeptide further comprises a second linker connecting the extracellular target binding domain and/or the extracellular TCR binding domain with the transmembrane domain. In some embodiments, the first linker and/or the second linker is a GS linker, an α-helical linker, a glycine-alanine polymer linker, an alanine-serine polymer linker, or an IgG4-Fc linker. In some embodiments, the first linker and/or the second linker comprises the amino acid sequence of any of SEQ ID NOs: 1-21, 67, and 68.

In some embodiments, there is provided a chimeric receptor polypeptide comprising (or consisting essentially of or consisting of), from the N-terminus to the C-terminus: a) an optional signal peptide; b) an extracellular target binding domain comprising a target antigen binding domain (such as scFv, sdAb, or DARPin) specifically binding to a target antigen (e.g., BCMA); c) an optional first linker (e.g., GS linker), d) an extracellular TCR binding domain comprising a TCR antigen binding domain (such as scFv or sdAb) specifically binding to a TCR subunit (e.g., extracellular domain of a TCR subunit, such as extracellular domain of CD3ε or TCR); e) an optional second linker (e.g., GS linker); f) an optional hinge region (e.g., CD8 hinge); g) a transmembrane domain comprising a transmembrane domain of a first TCR subunit; and h) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of), from the N-terminus to the C-terminus: a) an optional signal peptide; b) an extracellular TCR binding domain comprising a TCR antigen binding domain (such as scFv or sdAb) specifically binding to a TCR subunit (e.g., extracellular domain of a TCR subunit, such as extracellular domain of CD3ε or TCR); c) an optional first linker (e.g., GS linker), d) an extracellular target binding domain comprising a target antigen binding domain (such as scFv, sdAb, or DARPin) specifically binding to a target antigen (e.g., BCMA); e) an optional second linker (e.g., GS linker); f) an optional hinge region (e.g., CD8 hinge); g) a transmembrane domain comprising a transmembrane domain of a first TCR subunit; and h) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the first TCR subunit is CD3ε and/or the second TCR subunit is CD3ε. In some embodiments, the first TCR subunit is CD3y and/or the second TCR subunit is CD3y. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRy and/or the second TCR subunit is TCRy. In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are different. In some embodiments, the first TCR subunit and the second TCR subunit are both selected from the group consisting of TCRα, TCRβ, TCRy, and TCRδ and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an extracellular domain of the first and/or second TCR subunits. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the target antigen binding domain is an sdAb specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the TCR antigen binding domain (e.g., scFv, sdAb) specifically binds to CD3ε(e.g., N-terminus of CD3ε. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-TCR scFv comprising the amino acid sequence of SEQ ID NO: 27. In some embodiments, the extracellular TCR binding domain comprises two or more TCR antigen binding domains (e.g., scFv or sdAb) arranged in tandem. In some embodiments, the extracellular target binding domain comprises two or more target antigen binding domains (e.g., scFv, sdAb, or DARPin) arranged in tandem. In some embodiments, the first linker and/or the second linker is a GS linker, an α-helical linker, a glycine-alanine polymer linker, an alanine-serine polymer linker, or an IgG4-Fc linker. In some embodiments, the first linker and/or the second linker comprises the amino acid sequence of any of SEQ ID NOs: 1-21, 67, and 68.

In some embodiments, the extracellular target binding domain comprises two or more (such as two) target antigen binding domains (e.g., scFv, sdAb, DARPin), such as two or more target antigen binding domains (e.g., scFv, sdAb, DARPin) arranged in tandem. For example, in some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (e.g., BCMA); b) an extracellular TCR binding domain comprising a TCR antigen binding domain (such as scFv or sdAb) specifically binding to a TCR subunit (e.g., extracellular domain of a TCR subunit, such as extracellular domain of CD3ε or TCR); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the two or more target antigen binding domains are arranged in tandem. In some embodiments, the two or more target antigen binding domains are connected by one or more optional linkers. In some embodiments, the target antigen binding domain is an scFv, sdAb, or DARPin. In some embodiments, the first TCR subunit is CD3ε and/or the second TCR subunit is CD3ε. In some embodiments, the first TCR subunit is CD3γ and/or the second TCR subunit is CD3γ. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRy and/or the second TCR subunit is TCRy. In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are different. In some embodiments, the two or more target antigen binding domains bind to the same antigen or the same epitope on an antigen. In some embodiments, the two or more target antigen binding domains bind to different epitopes on the same antigen. In some embodiments, the two or more target antigen binding domains bind to different antigens (i.e., different epitopes of different target antigens). In some embodiments, the two or more target antigen binding domains are the same. In some embodiments, the two or more target antigen binding domains are different. In some embodiments, the first TCR subunit and the second TCR subunit are both selected from the group consisting of TCRα, TCRβ, TCRy, and TCRδ and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an extracellular domain of the first and/or second TCR subunits. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the chimeric receptor polypeptide further comprises a hinge region (e.g., CD8 hinge region) N-terminal to the transmembrane domain of the chimeric receptor polypeptide. In some embodiments, the chimeric receptor polypeptide comprises a linker between the extracellular target binding domain and the extracellular TCR binding domain. In some embodiments, the chimeric receptor polypeptide comprises a linker between the extracellular target binding domain (or the extracellular TCR binding domain) and the transmembrane domain. In some embodiments, the two or more target antigen binding domains are sdAbs specifically binding to BCMA, such as any of the anti-BCMA sdAbs disclosed in PCT/CN2016/094408 and PCT/CN2017/096938. In some embodiments, the two or more target antigen binding domains (sdAbs) specifically binding to BCMA are the same. In some embodiments, the two or more target antigen binding domains (sdAbs) specifically binding to BCMA are different. In some embodiments, the anti-BCMA sdAb comprises the amino acid sequence of SEQ ID NO: 26. In some embodiments, the TCR antigen binding domain (e.g., scFv, sdAb) specifically binds to CD3ε(e.g., N-terminus of CD3ε. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-TCR scFv comprising the amino acid sequence of SEQ ID NO: 27. In some embodiments, the linker is a GS linker, an α-helical linker, a glycine-alanine polymer linker, an alanine-serine polymer linker, or an IgG4-Fc linker. In some embodiments, the linker is selected from the group consisting of SEQ ID NOs: 1-21, 67, and 68. In some embodiments, the extracellular TCR binding domain comprises two or more TCR antigen binding domains (e.g., scFv or sdAb) arranged in tandem.

In some embodiments, the extracellular TCR binding domain comprises two or more (such as two) TCR antigen binding domains (e.g., scFv, sdAb), such as two or more TCR antigen binding domains arranged in tandem. For example, in some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising two or more (such as two) TCR antigen binding domains (such as scFv or sdAb), each specifically binding to a TCR subunit (e.g., extracellular domain of a TCR subunit, such as extracellular domain of CD3ε or TCR); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the two or more TCR antigen binding domains are arranged in tandem. In some embodiments, the two or more TCR antigen binding domains are connected by one or more optional linkers. In some embodiments, the TCR antigen binding domain is scFv or sdAb. In some embodiments, the first TCR subunit is CD3ε and/or the second TCR subunit is CD3ε. In some embodiments, the first TCR subunit is CD3y and/or the second TCR subunit is CD3y. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRy and/or the second TCR subunit is TCRy. In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are different. In some embodiments, the two or more TCR antigen binding domains bind to the same epitope on the TCR subunit. In some embodiments, the two or more TCR antigen binding domains bind to different epitopes on the same TCR subunit. In some embodiments, the two or more TCR antigen binding domains bind to a different TCR subunits (i.e., different epitopes of different TCR subunits). In some embodiments, the two or more TCR antigen binding domains are the same. In some embodiments, the two or more TCR antigen binding domains are different. In some embodiments, the TCR subunit(s) recognized by the two or more TCR antigen binding domains is/are the same as the first TCR subunit and the second TCR subunit. In some embodiments, the TCR subunit(s) recognized by the two or more TCR antigen binding domains is/are different from the first TCR subunit or the second TCR subunit. In some embodiments, the first TCR subunit and the second TCR subunit are both selected from the group consisting of TCRα, TCRβ, TCRy, and TCRδ and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an extracellular domain of the first and/or second TCR subunits. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the chimeric receptor polypeptide further comprises a hinge region (e.g., CD8 hinge region) N-terminal to the transmembrane domain of the chimeric receptor polypeptide. In some embodiments, the chimeric receptor polypeptide comprises a linker between the extracellular target binding domain and the extracellular TCR binding domain. In some embodiments, the chimeric receptor polypeptide comprises a linker between the extracellular target binding domain (or the extracellular TCR binding domain) and the transmembrane domain. In some embodiments, the target antigen binding domain is an sdAb specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the two or more (such as two) TCR antigen binding domains (e.g., scFv, sdAb) specifically bind to CD3ε(e.g., N-terminus of CD3ε. In some embodiments, the two or more (such as two) TCR antigen binding domains are anti-CD3 sdAbs comprising the amino acid sequence of SEQ ID NO: 22 or 23. In some embodiments, the two or more (such as two) TCR antigen binding domains are anti-CD3 scFvs comprising the amino acid sequence of SEQ ID NO: 24. In some embodiments, the two or more (such as two) TCR antigen binding domains are anti-TCR scFvs comprising the amino acid sequence of SEQ ID NO: 27. In some embodiments, one of the two or more TCR antigen binding domains is an anti-CD3 sdAb comprising the amino acid sequence of SEQ ID NO: 22 or 23, and the other of the two or more TCR antigen binding domains is an anti-CD3 scFv comprising the amino acid sequence of SEQ ID NO: 24. In some embodiments, one of the two or more TCR antigen binding domains is an anti-CD3 sdAb comprising the amino acid sequence of SEQ ID NO: 22 or 23, and the other of the two or more TCR antigen binding domains is an anti-TCR scFv comprising the amino acid sequence of SEQ ID NO: 27. In some embodiments, one of the two or more TCR antigen binding domains is an anti-CD3 scFv comprising the amino acid sequence of SEQ ID NO: 24 and the other of the two or more TCR antigen binding domains is an anti-TCR scFv comprising the amino acid sequence of SEQ ID NO: 27. In some embodiments, the two or more TCR antigen binding domains (e.g., scFv, sdAb) are the same. In some embodiments, the two or more TCR antigen binding domains (e.g., scFv, sdAb) are different (e.g., different structure, or bind to different epitopes). In some embodiments, the extracellular target binding domain comprises two or more (such as two) target antigen binding domains (e.g., scFv, sdAb, or DARPin) arranged in tandem. In some embodiments, the linker is a GS linker, an α-helical linker, a glycine-alanine polymer linker, an alanine-serine polymer linker, or an IgG4-Fc linker. In some embodiments, the linker is selected from the group consisting of SEQ ID NOs: 1-21, 67, and 68.

In some embodiments, the extracellular TCR binding domain comprises a TCR antigen binding domain (such as scFv or sdAb) specifically binding to CD3 (such as CD3ε, for example the N-terminus of CD3ε). For example, in some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain (such as scFv or sdAb) specifically binding to CD3 (such as CD3ε, for example the N-terminus of CD3ε); c) a transmembrane domain, and d) an intracellular domain comprising an intracellular domain of a TCR subunit, wherein the TCR subunit is selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain (such as scFv or sdAb) specifically binding to CD3 (such as CD3ε, for example the N-terminus of CD3ε); and c) a transmembrane domain comprising a transmembrane domain of a TCR subunit, wherein the TCR subunit is selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain (such as scFv or sdAb) specifically binding to CD3 (such as CD3ε, for example the N-terminus of CD3ε,); c) a transmembrane domain comprising a transmembrane domain of a TCR subunit, wherein the TCR subunit is selected from the group consisting of CD3ε, CD3y, CD3δ, TCRα, TCRβ, TCRy, and TCRδ, and d) an intracellular domain. In some embodiments, the chimeric receptor polypeptide does not comprise an extracellular domain of the TCR subunit (or an extracellular domain of any TCR subunit). In some embodiments, the TCR subunit is selected from the group consisting of TCRα, TCRβ, TCRy, and TCRδ and the chimeric receptor polypeptide does not comprise a variable region of the extracellular domain of the TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the TCR antigen binding domain is an anti-CD3 sdAb comprising the amino acid sequence of SEQ ID NO: 22 or 23. In some embodiments, the TCR antigen binding domain is an anti-CD3 sdAb comprising CDR1, CDR2, and CDR3 of the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-CD3 scFv comprising HC-CDR 1-3 and LC-CDR 1-3 of the amino acid sequence of SEQ ID NO: 24. Thus in some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain, and d) an intracellular domain comprising an intracellular domain of a TCR subunit, wherein the TCR subunit is selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; and c) a transmembrane domain comprising a transmembrane domain of a TCR subunit, wherein the TCR subunit is selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of a TCR subunit, wherein the TCR subunit is selected from the group consisting of CD3ε, CD3y, CD3δ, TCRα, TCRβ, TCRy, and TCRδ, and d) an intracellular domain.

In some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the first TCR subunit is CD3ε and/or the second TCR subunit is CD3ε. In some embodiments, the first TCR subunit is CD3y and/or the second TCR subunit is CD3y. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRy and/or the second TCR subunit is TCRy. In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are different. In some embodiments, the first TCR subunit and the second TCR subunit are both selected from the group consisting of TCRα, TCRβ, TCRy, and TCRδ and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an extracellular domain of the first and/or second TCR subunits. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-CD3 sdAb comprising CDR1, CDR2, and CDR3 of the amino acid sequence of SEQ ID NO: 22 or 23. In some embodiments, the TCR antigen binding domain is an anti-CD3 scFv comprising HC-CDR 1-3 and LC-CDR 1-3 of the amino acid sequence of SEQ ID NO: 24. Thus in some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRγ, and TCRδ.

In some embodiments, there is provided a chimeric receptor polypeptide comprising (or consisting essentially of or consisting of), from the N-terminus to the C-terminus: a) an optional signal peptide; b) an extracellular target binding domain (e.g., anti-BCMA sdAb); c) an optional first linker (e.g., GS linker), d) an extracellular TCR binding domain comprising a TCR antigen binding domain (such as scFv or sdAb) specifically binding to CD3 (such as CD3ε, for example the N-terminus of CD3ε); e) an optional second linker (e.g., GS linker); f) an optional hinge region (e.g., CD8 hinge); g) a transmembrane domain comprising a transmembrane domain of a first TCR subunit; and h) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of), from the N-terminus to the C-terminus: a) an optional signal peptide; b) an extracellular TCR binding domain comprising a TCR antigen binding domain (such as scFv or sdAb) specifically binding to CD3 (such as CD3ε, for example the N-terminus of CD3ε); c) an optional first linker (e.g., GS linker), d) an extracellular target binding domain (e.g., anti-BCMA sdAb); e) an optional second linker (e.g., GS linker); f) an optional hinge region (e.g., CD8 hinge); g) a transmembrane domain comprising a transmembrane domain of a first TCR subunit; and h) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-CD3 sdAb comprising CDR1, CDR2, and CDR3 of the amino acid sequence of SEQ ID NO: 22 or 23. In some embodiments, the TCR antigen binding domain is an anti-CD3 scFv comprising HC-CDR 1-3 and LC-CDR 1-3 of the amino acid sequence of SEQ ID NO: 24. Thus in some embodiments, there is provided a chimeric receptor polypeptide comprising (or consisting essentially of or consisting of), from the N-terminus to the C-terminus: a) an optional signal peptide; b) an extracellular target binding domain (e.g., anti-BCMA sdAb); c) an optional first linker (e.g., GS linker), d) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; e) an optional second linker (e.g., GS linker); f) an optional hinge region (e.g., CD8 hinge); g) a transmembrane domain comprising a transmembrane domain of a first TCR subunit; and h) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of), from the N-terminus to the C-terminus: a) an optional signal peptide; b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) an optional first linker (e.g., GS linker), d) an extracellular target binding domain (e.g., anti-BCMA sdAb); e) an optional second linker (e.g., GS linker); f) an optional hinge region (e.g., CD8 hinge); g) a transmembrane domain comprising a transmembrane domain of a first TCR subunit; and h) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the first TCR subunit is CD3ε and/or the second TCR subunit is CD3ε. In some embodiments, the first TCR subunit is CD3γ and/or the second TCR subunit is CD3γ. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRy and/or the second TCR subunit is TCRy. In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are different. In some embodiments, the first TCR subunit and the second TCR subunit are both selected from the group consisting of TCRα, TCRβ, TCRy, and TCRδ and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an extracellular domain of the first and/or second TCR subunits. In some embodiments, the chimeric receptor polypeptide does not comprise an extracellular domain of any TCR subunits. In some embodiments, the chimeric receptor polypeptide does not comprise an intracellular co-stimulatory domain.

In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to CD3 (such as CD3ε, for example the N-terminus of CD3ε); c) a transmembrane domain, and d) an intracellular domain comprising an intracellular domain of a TCR subunit, wherein the TCR subunit is selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to CD3 (such as CD3ε, for example the N-terminus of CD3ε); and c) a transmembrane domain comprising a transmembrane domain of a TCR subunit, wherein the TCR subunit is selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of a TCR subunit, wherein the TCR subunit is selected from the group consisting of CD3ε, CD3y, CD3δ, TCRα, TCRβ, TCRy, and TCRδ, and d) an intracellular domain. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-CD3 sdAb comprising CDR1, CDR2, and CDR3 of the amino acid sequence of SEQ ID NO: 22 or 23. In some embodiments, the TCR antigen binding domain is an anti-CD3 scFv comprising HC-CDR 1-3 and LC-CDR 1-3 of the amino acid sequence of SEQ ID NO: 24. Thus in some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain, and d) an intracellular domain comprising an intracellular domain of a TCR subunit, wherein the TCR subunit is selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; and c) a transmembrane domain comprising a transmembrane domain of a TCR subunit, wherein the TCR subunit is selected from the group consisting of CD3ε, CD3y, CD3δ, TCRα, TCRβ, TCRy, and TCRδ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of a TCR subunit, wherein the TCR subunit is selected from the group consisting of CD3ε, CD3y, CD3δ, TCRα, TCRβ, TCRγ, and TCRδ, and d) an intracellular domain. In some embodiments, the TCR subunit is selected from the group consisting of TCRα, TCRβ, TCRγ, and TCRδ and the chimeric receptor polypeptide does not comprise a variable region of the extracellular domain of the TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an extracellular domain of the TCR subunit (or an extracellular domain of any TCR subunit). In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the target antigen binding domain is an sdAb specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the extracellular TCR binding domain comprises two or more TCR antigen binding domains (e.g., scFv or sdAb) arranged in tandem. In some embodiments, the extracellular target binding domain comprises two or more target antigen binding domains (e.g., scFv, sdAb, or DARPin) arranged in tandem. In some embodiments, the chimeric receptor polypeptide further comprises a hinge region (e.g., CD8 hinge region) N-terminal to the transmembrane domain of the chimeric receptor polypeptide. In some embodiments, the chimeric receptor polypeptide further comprises a first linker connecting the extracellular target binding domain with the extracellular TCR binding domain. In some embodiments, the chimeric receptor polypeptide further comprises a second linker connecting the extracellular target binding domain and/or the extracellular TCR binding domain with the transmembrane domain. In some embodiments, the first linker and/or the second linker is a GS linker, an α-helical linker, a glycine-alanine polymer linker, an alanine-serine polymer linker, or an IgG4-Fc linker. In some embodiments, the first linker and/or the second linker comprises the amino acid sequence of any of SEQ ID NOs: 1-21, 67, and 68.

In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-CD3 sdAb comprising CDR1, CDR2, and CDR3 of the amino acid sequence of SEQ ID NO: 22 or 23. In some embodiments, the TCR antigen binding domain is an anti-CD3 scFv comprising HC-CDR 1-3 and LC-CDR 1-3 of the amino acid sequence of SEQ ID NO: 24. Thus in some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the first TCR subunit is CD3ε and/or the second TCR subunit is CD3ε. In some embodiments, the first TCR subunit is CD3y and/or the second TCR subunit is CD3y. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRy and/or the second TCR subunit is TCRy. In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are different. In some embodiments, the first TCR subunit and the second TCR subunit are both selected from the group consisting of TCRα, TCRβ, TCRy, and TCRδ and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an extracellular domain of the first and/or second TCR subunits. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the target antigen binding domain is an sdAb specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the extracellular TCR binding domain comprises two or more TCR antigen binding domains (e.g., scFv or sdAb) arranged in tandem. In some embodiments, the extracellular target binding domain comprises two or more target antigen binding domains (e.g., scFv, sdAb, or DARPin) arranged in tandem. In some embodiments, the chimeric receptor polypeptide further comprises a hinge region (e.g., CD8 hinge region) N-terminal to the transmembrane domain of the chimeric receptor polypeptide. In some embodiments, the chimeric receptor polypeptide further comprises a first linker connecting the extracellular target binding domain with the extracellular TCR binding domain. In some embodiments, the chimeric receptor polypeptide further comprises a second linker connecting the extracellular target binding domain and/or the extracellular TCR binding domain with the transmembrane domain. In some embodiments, the first linker and/or the second linker is a GS linker, an α-helical linker, a glycine-alanine polymer linker, an alanine-serine polymer linker, or an IgG4-Fc linker. In some embodiments, the first linker and/or the second linker comprises the amino acid sequence of any of SEQ ID NOs: 1-21, 67, and 68.

In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (e.g., BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-CD3 sdAb comprising CDR1, CDR2, and CDR3 of the amino acid sequence of SEQ ID NO: 22 or 23. In some embodiments, the TCR antigen binding domain is an anti-CD3 scFv comprising HC-CDR 1-3 and LC-CDR 1-3 of the amino acid sequence of SEQ ID NO: 24. Thus in some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (e.g., BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3y, CD3δ, TCRα, TCRβ, TCRy, and TCRδ. In some embodiments, the first TCR subunit is CD3ε and/or the second TCR subunit is CD3ε. In some embodiments, the first TCR subunit is CD3y and/or the second TCR subunit is CD3y. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRy and/or the second TCR subunit is TCRy. In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are different. In some embodiments, the two or more (such as two) target antigen binding domains are arranged in tandem. In some embodiments, the two or more (such as two) target antigen binding domains bind to the same antigen or the same epitope on an antigen. In some embodiments, the two or more (such as two) target antigen binding domains bind to different epitopes on the same antigen. In some embodiments, the two or more (such as two) target antigen binding domains bind to different antigens. In some embodiments, the two or more (such as two) target antigen binding domains are the same. In some embodiments, the two or more (such as two) target antigen binding domains are different. In some embodiments, the first TCR subunit and the second TCR subunit are both selected from the group consisting of TCRα, TCRβ, TCRy, and TCRδ and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRy, and TCRδ. In some embodiments, the chimeric receptor polypeptide does not comprise an extracellular domain of the first and/or second TCR subunits. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the chimeric receptor polypeptide comprises a linker between the extracellular target binding domain and the extracellular TCR binding domain. In some embodiments, the chimeric receptor polypeptide comprises a linker between the extracellular target binding domain (or the extracellular TCR binding domain) and the transmembrane domain. In some embodiments, the extracellular target binding domain comprises two or more target antigen binding domains (e.g., scFv, sdAb, or DARPin) arranged in tandem. In some embodiments, the chimeric receptor polypeptide further comprises a hinge region (e.g., CD8 hinge region) N-terminal to the transmembrane domain of the chimeric receptor polypeptide. In some embodiments, the chimeric receptor polypeptide comprises a linker between the two or more (such as two) target antigen binding domains within the extracellular target binding domain. In some embodiments, the linker is a GS linker, an α-helical linker, a glycine-alanine polymer linker, an alanine-serine polymer linker, or an IgG4-Fc linker. In some embodiments, the linker comprises the amino acid sequence of any of SEQ ID NOs: 1-21, 67, and 68. In some embodiments, the two or more (e.g., two) target antigen binding domains are sdAbs specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26.

In some embodiments, the first TCR subunit and the second TCR subunits are selected from the group consisting of CD3ε, CD3γ, and CD3δ. For example, in some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of CD3ε; and d) an intracellular domain comprising an intracellular domain of CD3ε. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of CD3y; and d) an intracellular domain comprising an intracellular domain of CD3y. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of CD3δ; and d) an intracellular domain comprising an intracellular domain of CD3δ. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-CD3 sdAb comprising CDR1, CDR2, and CDR3 of the amino acid sequence of SEQ ID NO: 22 or 23. In some embodiments, the TCR antigen binding domain is an anti-CD3 scFv comprising HC-CDR 1-3 and LC-CDR 1-3 of the amino acid sequence of SEQ ID NO: 24. Thus in some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of CD3ε; and d) an intracellular domain comprising an intracellular domain of CD3ε. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of CD3y; and d) an intracellular domain comprising an intracellular domain of CD3y. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of CD3δ; and d) an intracellular domain comprising an intracellular domain of CD3δ. In some embodiments, the chimeric receptor polypeptide does not comprise an extracellular domain of CD3ε. In some embodiments, the chimeric receptor polypeptide does not comprise an extracellular domain of CD3y. In some embodiments, the chimeric receptor polypeptide does not comprise an extracellular domain of CD3δ. In some embodiments, the chimeric receptor polypeptide does not comprise an extracellular domain of any TCR subunits.In some embodiments, the first TCR subunit and the second TCR subunits are selected from the group consisting of TCRα, TCRβ, TCRy, and TCRδ. For example, in some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of TCRα; and d) an intracellular domain comprising an intracellular domain of TCRα. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of TCRβ; and d) an intracellular domain comprising an intracellular domain of TCRβ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of TCRy; and d) an intracellular domain comprising an intracellular domain of TCRy. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of TCRδ; and d) an intracellular domain comprising an intracellular domain of TCRδ. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-CD3 sdAb comprising CDR1, CDR2, and CDR3 of the amino acid sequence of SEQ ID NO: 22 or 23. In some embodiments, the TCR antigen binding domain is an anti-CD3 scFv comprising HC-CDR 1-3 and LC-CDR 1-3 of the amino acid sequence of SEQ ID NO: 24. Thus in some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of TCRα; and d) an intracellular domain comprising an intracellular domain of TCRα. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of TCRβ; and d) an intracellular domain comprising an intracellular domain of TCRβ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of TCRγ; and d) an intracellular domain comprising an intracellular domain of TCRγ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of TCRδ; and d) an intracellular domain comprising an intracellular domain of TCRδ. In some embodiments, the chimeric receptor polypeptide does not comprise the variable region of TCRα but comprises the constant region of TCRα (e.g., comprises the TCRα constant region N-terminal to the TCRα transmembrane domain and intracellular domain). In some embodiments, the chimeric receptor polypeptide does not comprise the variable region of TCRβ but comprises the constant region of TCRβ (e.g., comprises the TCRβ constant region N-terminal to the TCRβ transmembrane domain and intracellular domain). In some embodiments, the chimeric receptor polypeptide does not comprise the variable region of TCRγ but comprises the constant region of TCRγ (e.g., comprises the TCRγ constant region N-terminal to the TCRγ transmembrane domain and intracellular domain). In some embodiments, the chimeric receptor polypeptide does not comprise the variable region of TCRδ but comprises the constant region of TCRδ (e.g., comprises the TCRδ constant region N-terminal to the TCRδ transmembrane domain and intracellular domain). In some embodiments, the chimeric receptor polypeptide does not comprise an extracellular domain of TCRα. In some embodiments, the chimeric receptor polypeptide does not comprise an extracellular domain of TCRβ. In some embodiments, the chimeric receptor polypeptide does not comprise an extracellular domain of TCRγ. In some embodiments, the chimeric receptor polypeptide does not comprise an extracellular domain of TCRδ. In some embodiments, the chimeric receptor polypeptide does not comprise an extracellular domain of any of TCRα, TCRβ, TCRy, and TCRδ. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the extracellular TCR binding domain comprises two or more TCR antigen binding domains (e.g., scFv or sdAb) arranged in tandem. In some embodiments, the extracellular target binding domain comprises two or more target antigen binding domains (e.g., scFv, sdAb, or DARPin) arranged in tandem. In some embodiments, the chimeric receptor polypeptide further comprises a hinge region (e.g., CD8 hinge region) N-terminal to the transmembrane domain of the chimeric receptor polypeptide. In some embodiments, the extracellular target binding domain comprises an sdAb specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the chimeric receptor polypeptide further comprises a first linker connecting the extracellular target binding domain with the extracellular TCR binding domain. In some embodiments, the chimeric receptor polypeptide further comprises a second linker connecting the extracellular target binding domain and/or the extracellular TCR binding domain with the transmembrane domain. In some embodiments, the first linker and/or the second linker is a GS linker, an α-helical linker, a glycine-alanine polymer linker, an alanine-serine polymer linker, or an IgG4-Fc linker. In some embodiments, the first linker and/or the second linker comprises the amino acid sequence of any of SEQ ID NOs: 1-21, 67, and 68.

In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of CD3ε; and d) an intracellular domain comprising an intracellular domain of CD3ε. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of CD3γ; and d) an intracellular domain comprising an intracellular domain of CD3γ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of CD3δ; and d) an intracellular domain comprising an intracellular domain of CD3δ. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-CD3 sdAb comprising CDR1, CDR2, and CDR3 of the amino acid sequence of SEQ ID NO: 22 or 23. In some embodiments, the TCR antigen binding domain is an anti-CD3 scFv comprising HC-CDR 1-3 and LC-CDR 1-3 of the amino acid sequence of SEQ ID NO: 24. Thus in some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of CD3ε; and d) an intracellular domain comprising an intracellular domain of CD3ε. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of CD3γ; and d) an intracellular domain comprising an intracellular domain of CD3γ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of CD3δ; and d) an intracellular domain comprising an intracellular domain of CD3δ. In some embodiments, the chimeric receptor polypeptide does not comprise an extracellular domain of CD3ε. In some embodiments, the chimeric receptor polypeptide does not comprise an extracellular domain of CD3γ. In some embodiments, the chimeric receptor polypeptide does not comprise an extracellular domain of CD3δ. In some embodiments, the chimeric receptor polypeptide does not comprise an extracellular domain of any TCR subunits. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of TCRα; and d) an intracellular domain comprising an intracellular domain of TCRα. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of TCRβ; and d) an intracellular domain comprising an intracellular domain of TCRβ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of TCRγ; and d) an intracellular domain comprising an intracellular domain of TCRγ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of TCRδ; and d) an intracellular domain comprising an intracellular domain of TCRδ. In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-CD3 sdAb comprising CDR1, CDR2, and CDR3 of the amino acid sequence of SEQ ID NO: 22 or 23. In some embodiments, the TCR antigen binding domain is an anti-CD3 scFv comprising HC-CDR 1-3 and LC-CDR 1-3 of the amino acid sequence of SEQ ID NO: 24. Thus in some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of TCRα; and d) an intracellular domain comprising an intracellular domain of TCRα. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of TCRβ; and d) an intracellular domain comprising an intracellular domain of TCRβ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of TCRγ; and d) an intracellular domain comprising an intracellular domain of TCRγ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of TCRδ; and d) an intracellular domain comprising an intracellular domain of TCRδ. In some embodiments, the chimeric receptor polypeptide does not comprise the variable region of TCRα but comprises the constant region of TCRα (e.g., comprises the TCRα constant region N-terminal to the TCRα transmembrane domain and intracellular domain). In some embodiments, the chimeric receptor polypeptide does not comprise the variable region of TCRβ but comprises the constant region of TCRβ (e.g., comprises the TCRβ constant region N-terminal to the TCRβ transmembrane domain and intracellular domain). In some embodiments, the chimeric receptor polypeptide does not comprise the variable region of TCRγ but comprises the constant region of TCRγ (e.g., comprises the TCRγ constant region N-terminal to the TCRγ transmembrane domain and intracellular domain). In some embodiments, the chimeric receptor polypeptide does not comprise the variable region of TCRδ but comprises the constant region of TCRδ (e.g., comprises the TCRδ constant region N-terminal to the TCRδ transmembrane domain and intracellular domain). In some embodiments, the chimeric receptor polypeptide does not comprise an extracellular domain of TCRα. In some embodiments, the chimeric receptor polypeptide does not comprise an extracellular domain of TCRβ. In some embodiments, the chimeric receptor polypeptide does not comprise an extracellular domain of TCRγ. In some embodiments, the chimeric receptor polypeptide does not comprise an extracellular domain of TCRδ. In some embodiments, the chimeric receptor polypeptide does not comprise an extracellular domain of any of TCRα, TCRβ, TCRγ, and TCRδ. In some embodiments, the chimeric receptor polypeptide does not comprise an extracellular domain of any TCR subunits. In some embodiments, the two or more target antigen binding domains bind to the same antigen or the same epitope on an antigen. In some embodiments, the two or more target antigen binding domains bind to different epitopes on the same antigen. In some embodiments, the two or more target antigen binding domains bind to different antigens. In some embodiments, the two or more target antigen binding domains are the same. In some embodiments, the two or more target antigen binding domains are different. In some embodiments, the chimeric receptor polypeptide does not comprise an intracellular co-stimulatory domain. In some embodiments, the two or more target antigen binding domains are arranged in tandem. In some embodiments, the two or more target antigen binding domains are connected by one or more optional linkers. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the extracellular TCR binding domain comprises two or more TCR antigen binding domains (e.g., scFv or sdAb) arranged in tandem. In some embodiments, the chimeric receptor polypeptide further comprises a hinge region (e.g., CD8 hinge region) N-terminal to the transmembrane domain of the chimeric receptor polypeptide. In some embodiments, the chimeric receptor polypeptide comprises a linker between the extracellular target binding domain and the extracellular TCR binding domain. In some embodiments, the chimeric receptor polypeptide comprises a linker between the extracellular target binding domain (or extracellular TCR binding domain) and the transmembrane domain. In some embodiments, the chimeric receptor polypeptide comprises a linker between the two or more target antigen binding domains within the extracellular target binding domain. In some embodiments, the two or more target antigen binding domains are sdAbs specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the liner is a GS linker, an α-helical linker, a glycine-alanine polymer linker, an alanine-serine polymer linker, or an IgG4-Fc linker. In some embodiments, the two or more target antigen binding domains are connected by a linker selected from the group consisting of SEQ ID NOs: 1-21, 67, and 68.

In some embodiments, the extracellular TCR binding domain comprises a TCR antigen binding domain (such as scFv or sdAb) specifically binding to TCR (such as TCRγ/δ, for example the constant region of TCRγ/δ). For example, in some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain (such as scFv or sdAb) specifically binding to TCR (such as TCRγ/δ, for example the constant region of TCRγ/δ); c) a transmembrane domain, and d) an intracellular domain comprising an intracellular domain of a TCR subunit, wherein the TCR subunit is selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain (such as scFv or sdAb) specifically binding to TCR (such as TCRγ/δ, for example the constant region of TCRγ/δ); and c) a transmembrane domain comprising a transmembrane domain of a TCR subunit, wherein the TCR subunit is selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of a TCR subunit, wherein the TCR subunit is selected from the group consisting of CD3ε, CD3γ, CD3δ, TCRα, TCRβ, TCRγ, and TCRδ, and d) an intracellular domain. In some embodiments, the chimeric receptor polypeptide does not comprise an extracellular domain of the TCR subunit (or an extracellular domain of any TCR subunit). In some embodiments, the TCR subunit is selected from the group consisting of TCRα, TCRβ, TCRγ, and TCRδ, and the chimeric receptor polypeptide does not comprise a variable region of the extracellular domain of the TCR subunit. In some embodiments, the chimeric receptor polypeptide does not comprise a variable region of the extracellular domain of any of TCRα, TCRβ, TCRγ, and TCRδ. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the TCR antigen binding domain is an anti-TCR scFv comprising the amino acid sequence of SEQ ID NO: 27. In some embodiments, the TCR antigen binding domain is an anti-TCR scFv comprising HC-CDR 1-3 and LC-CDR 1-3 of the amino acid sequence of SEQ ID NO: 27. Thus in some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain, and d) an intracellular domain comprising an intracellular domain of a TCR subunit, wherein the TCR subunit is selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain specifically binding to TCRγ/δ, wherein the TCR antigen binding domain is an scFv comprising the amino acid sequence of SEQ ID NO: 27; and c) a transmembrane domain comprising a transmembrane domain of a TCR subunit, wherein the TCR subunit is selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of a TCR subunit, wherein the TCR subunit is selected from the group consisting of CD3ε, CD3γ, CD3δ, TCRα, TCRβ, TCRγ, and TCRδ, and d) an intracellular domain.

In some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, the TCR antigen binding domain is an anti-TCR scFv comprising the amino acid sequence of SEQ ID NO: 27. In some embodiments, the TCR antigen binding domain is an anti-TCR scFv comprising HC-CDR 1-3 and LC-CDR 1-3 of the amino acid sequence of SEQ ID NO: 27. Thus in some embodiments, there is provided a chimeric receptor polypeptide comprising (or consisting essentially of or consisting of): a) an extracellular target binding domain (e.g., anti-BCMA sdAb); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, the TCR antigen binding domain is an anti-TCR scFv comprising the amino acid sequence of SEQ ID NO: 27. In some embodiments, the TCR antigen binding domain is an anti-TCR scFv comprising HC-CDR 1-3 and LC-CDR 1-3 of the amino acid sequence of SEQ ID NO: 27. Thus in some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (e.g., BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, the TCR antigen binding domain is an anti-TCR scFv comprising the amino acid sequence of SEQ ID NO: 27. In some embodiments, the TCR antigen binding domain is an anti-TCR scFv comprising HC-CDR 1-3 and LC-CDR 1-3 of the amino acid sequence of SEQ ID NO: 27. Thus in some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (e.g., BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, the two or more (such as two) target antigen binding domains are arranged in tandem. In some embodiments, the two or more (such as two) target antigen binding domains bind to the same antigen or the same epitope on an antigen. In some embodiments, the two or more (such as two) target antigen binding domains bind to different epitopes on the same antigen. In some embodiments, the two or more (such as two) target antigen binding domains bind to different antigens. In some embodiments, the two or more (such as two) target antigen binding domains are the same. In some embodiments, the two or more (such as two) target antigen binding domains are different. In some embodiments, the first TCR subunit is CD3ε and/or the second TCR subunit is CD3ε. In some embodiments, the first TCR subunit is CD3γ and/or the second TCR subunit is CD3γ. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRγ and/or the second TCR subunit is TCRγ. In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are different. In some embodiments, the first TCR subunit and the second TCR subunit are both selected from the group consisting of TCRα, TCRβ, TCRγ, and TCRδ, and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRγ, and TCRδ. In some embodiments, the chimeric receptor polypeptide does not comprise an extracellular domain of the first and/or second TCR subunits. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the target antigen binding domain is an sdAb specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the extracellular TCR binding domain comprises two or more TCR antigen binding domains (e.g., scFv or sdAb) arranged in tandem. In some embodiments, the extracellular target binding domain comprises two or more target antigen binding domains (e.g., scFv, sdAb, or DARPin) arranged in tandem. In some embodiments, the chimeric receptor polypeptide further comprises a hinge region (e.g., CD8 hinge region) N-terminal to the transmembrane domain of the chimeric receptor polypeptide. In some embodiments, the chimeric receptor polypeptide further comprises a first linker connecting the extracellular target binding domain with the extracellular TCR binding domain. In some embodiments, the chimeric receptor polypeptide further comprises a second linker connecting the extracellular target binding domain and/or the extracellular TCR binding domain with the transmembrane domain. In some embodiments, the first linker and/or the second linker is a GS linker, an α-helical linker, a glycine-alanine polymer linker, an alanine-serine polymer linker, or an IgG4-Fc linker. In some embodiments, the first linker and/or the second linker comprises the amino acid sequence of any of SEQ ID NOs: 1-21, 67, and 68.

In some embodiments, the first TCR subunit and the second TCR subunits are selected from the group consisting of CD3ε, CD3γ, and CD3δ. For example, in some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of CD3ε; and d) an intracellular domain comprising an intracellular domain of CD3ε. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of CD3γ; and d) an intracellular domain comprising an intracellular domain of CD3γ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of CD3δ; and d) an intracellular domain comprising an intracellular domain of CD3δ. In some embodiments, the TCR antigen binding domain is an anti-TCR scFv comprising the amino acid sequence of SEQ ID NO: 27. In some embodiments, the TCR antigen binding domain is an anti-TCR scFv comprising HC-CDR 1-3 and LC-CDR 1-3 of the amino acid sequence of SEQ ID NO: 27. Thus in some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of CD3ε; and d) an intracellular domain comprising an intracellular domain of CD3ε. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of CD3γ; and d) an intracellular domain comprising an intracellular domain of CD3γ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of CD3δ; and d) an intracellular domain comprising an intracellular domain of CD3δ. In some embodiments, the chimeric receptor polypeptide does not comprise an extracellular domain of CD3ε. In some embodiments, the chimeric receptor polypeptide does not comprise an extracellular domain of CD3γ. In some embodiments, the chimeric receptor polypeptide does not comprise an extracellular domain of CD3δ. In some embodiments, the chimeric receptor polypeptide does not comprise an extracellular domain of any TCR subunits. In some embodiments, the first TCR subunit and the second TCR subunits are selected from the group consisting of TCRα, TCRβ, TCRγ, and TCRδ. For example, in some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of TCRα; and d) an intracellular domain comprising an intracellular domain of TCRα. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of TCRβ; and d) an intracellular domain comprising an intracellular domain of TCRβ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of TCRγ; and d) an intracellular domain comprising an intracellular domain of TCRγ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of TCRδ; and d) an intracellular domain comprising an intracellular domain of TCRδ. In some embodiments, the TCR antigen binding domain is an anti-TCR scFv comprising the amino acid sequence of SEQ ID NO: 27. In some embodiments, the TCR antigen binding domain is an anti-TCR scFv comprising HC-CDR 1-3 and LC-CDR 1-3 of the amino acid sequence of SEQ ID NO: 27. Thus in some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of TCRα; and d) an intracellular domain comprising an intracellular domain of TCRα. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of TCRβ; and d) an intracellular domain comprising an intracellular domain of TCRβ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of TCRγ; and d) an intracellular domain comprising an intracellular domain of TCRγ. In some embodiments, there is provided a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of TCRδ; and d) an intracellular domain comprising an intracellular domain of TCRδ. In some embodiments, the chimeric receptor polypeptide does not comprise the variable region of TCRα but comprises the constant region of TCRα (e.g., comprises the TCRα constant region N-terminal to the TCRα transmembrane domain and intracellular domain). In some embodiments, the chimeric receptor polypeptide does not comprise the variable region of TCRβ but comprises the constant region of TCRβ (e.g., comprises the TCRβ constant region N-terminal to the TCRβ transmembrane domain and intracellular domain). In some embodiments, the chimeric receptor polypeptide does not comprise the variable region of TCRγ but comprises the constant region of TCRγ (e.g., comprises the TCRγ constant region N-terminal to the TCRγ transmembrane domain and intracellular domain). In some embodiments, the chimeric receptor polypeptide does not comprise the variable region of TCRδ but comprises the constant region of TCRδ (e.g., comprises the TCRδ constant region N-terminal to the TCRδ transmembrane domain and intracellular domain). In some embodiments, the chimeric receptor polypeptide does not comprise an extracellular domain of TCRα. In some embodiments, the chimeric receptor polypeptide does not comprise an extracellular domain of TCRβ. In some embodiments, the chimeric receptor polypeptide does not comprise an extracellular domain of TCRγ. In some embodiments, the chimeric receptor polypeptide does not comprise an extracellular domain of TCRδ. In some embodiments, the chimeric receptor polypeptide does not comprise an extracellular domain of any of TCRα, TCRβ, TCRγ, and TCRδ. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the extracellular TCR binding domain comprises two or more TCR antigen binding domains (e.g., scFv or sdAb) arranged in tandem. In some embodiments, the extracellular target binding domain comprises two or more target antigen binding domains (e.g., scFv, sdAb, or DARPin) arranged in tandem. In some embodiments, the chimeric receptor polypeptide further comprises a hinge region (e.g., CD8 hinge region) N-terminal to the transmembrane domain of the chimeric receptor polypeptide. In some embodiments, the extracellular target binding domain comprises an sdAb specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the chimeric receptor polypeptide further comprises a first linker connecting the extracellular target binding domain with the extracellular TCR binding domain. In some embodiments, the chimeric receptor polypeptide further comprises a second linker connecting the extracellular target binding domain and/or the extracellular TCR binding domain with the transmembrane domain. In some embodiments, the first linker and/or the second linker is a GS linker, an α-helical linker, a glycine-alanine polymer linker, an alanine-serine polymer linker, or an IgG4-Fc linker. In some embodiments, the first linker and/or the second linker comprises the amino acid sequence of any of SEQ ID NOs: 1-21, 67, and 68.

In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of CD3ε; and d) an intracellular domain comprising an intracellular domain of CD3ε. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of CD3γ; and d) an intracellular domain comprising an intracellular domain of CD3γ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of CD3δ; and d) an intracellular domain comprising an intracellular domain of CD3δ. In some embodiments, the TCR antigen binding domain is an anti-TCR scFv comprising the amino acid sequence of SEQ ID NO: 27. In some embodiments, the TCR antigen binding domain is an anti-TCR scFv comprising HC-CDR 1-3 and LC-CDR 1-3 of the amino acid sequence of SEQ ID NO: 27. Thus in some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of CD3ε; and d) an intracellular domain comprising an intracellular domain of CD3ε. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of CD3γ; and d) an intracellular domain comprising an intracellular domain of CD3γ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of CD3δ; and d) an intracellular domain comprising an intracellular domain of CD3δ. In some embodiments, the chimeric receptor polypeptide does not comprise an extracellular domain of CD3ε. In some embodiments, the chimeric receptor polypeptide does not comprise an extracellular domain of CD3γ. In some embodiments, the chimeric receptor polypeptide does not comprise an extracellular domain of CD3δ. In some embodiments, the chimeric receptor polypeptide does not comprise an extracellular domain of any TCR subunits. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of TCRα; and d) an intracellular domain comprising an intracellular domain of TCRα. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of TCRβ; and d) an intracellular domain comprising an intracellular domain of TCRβ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of TCRγ; and d) an intracellular domain comprising an intracellular domain of TCRγ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of TCRδ; and d) an intracellular domain comprising an intracellular domain of TCRδ. In some embodiments, the TCR antigen binding domain is an anti-TCR scFv comprising the amino acid sequence of SEQ ID NO: 27. In some embodiments, the TCR antigen binding domain is an anti-TCR scFv comprising HC-CDR 1-3 and LC-CDR 1-3 of the amino acid sequence of SEQ ID NO: 27. Thus in some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of TCRα; and d) an intracellular domain comprising an intracellular domain of TCRα. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of TCRβ; and d) an intracellular domain comprising an intracellular domain of TCRβ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of TCRγ; and d) an intracellular domain comprising an intracellular domain of TCRγ. In some embodiments, there is provided a chimeric receptor polypeptide comprising (or consisting essentially of or consisting of): a) an extracellular target binding domain comprising two or more (such as two) target antigen binding domains (such as scFv, sdAb, or DARPin), each specifically binding to a target antigen (such as BCMA); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of TCRδ; and d) an intracellular domain comprising an intracellular domain of TCRδ. In some embodiments, the chimeric receptor polypeptide does not comprise the variable region of TCRα but comprises the constant region of TCRα (e.g., comprises the TCRα constant region N-terminal to the TCRα transmembrane domain and intracellular domain). In some embodiments, the chimeric receptor polypeptide does not comprise the variable region of TCRβ but comprises the constant region of TCRβ (e.g., comprises the TCRβ constant region N-terminal to the TCRβ transmembrane domain and intracellular domain). In some embodiments, the chimeric receptor polypeptide does not comprise the variable region of TCRγ but comprises the constant region of TCRγ (e.g., comprises the TCRγ constant region N-terminal to the TCRγ transmembrane domain and intracellular domain). In some embodiments, the chimeric receptor polypeptide does not comprise the variable region of TCRδ but comprises the constant region of TCRδ (e.g., comprises the TCRδ constant region N-terminal to the TCRδ transmembrane domain and intracellular domain). In some embodiments, the chimeric receptor polypeptide does not comprise an extracellular domain of TCRα. In some embodiments, the chimeric receptor polypeptide does not comprise an extracellular domain of TCRβ. In some embodiments, the chimeric receptor polypeptide does not comprise an extracellular domain of TCRγ. In some embodiments, the chimeric receptor polypeptide does not comprise an extracellular domain of TCRδ. In some embodiments, the chimeric receptor polypeptide does not comprise an extracellular domain of any of TCRα, TCRβ, TCRγ, and TCRδ. In some embodiments, the chimeric receptor polypeptide does not comprise an extracellular domain of any TCR subunits. In some embodiments, the two or more target antigen binding domains bind to the same antigen or the same epitope on an antigen. In some embodiments, the two or more target antigen binding domains bind to different epitopes on the same antigen. In some embodiments, the two or more target antigen binding domains bind to different antigens. In some embodiments, the two or more target antigen binding domains are the same. In some embodiments, the two or more target antigen binding domains are different. In some embodiments, the chimeric receptor polypeptide does not comprise an intracellular co-stimulatory domain. In some embodiments, the two or more target antigen binding domains are arranged in tandem. In some embodiments, the two or more target antigen binding domains are connected by one or more optional linkers. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the extracellular TCR binding domain comprises two or more TCR antigen binding domains (e.g., scFv or sdAb) arranged in tandem. In some embodiments, the chimeric receptor polypeptide further comprises a hinge region (e.g., CD8 hinge region) N-terminal to the transmembrane domain of the chimeric receptor polypeptide. In some embodiments, the chimeric receptor polypeptide comprises a linker between the extracellular target binding domain and the extracellular TCR binding domain. In some embodiments, the chimeric receptor polypeptide comprises a linker between the extracellular target binding domain (or extracellular TCR binding domain) and the transmembrane domain. In some embodiments, the chimeric receptor polypeptide comprises a linker between the two or more target antigen binding domains within the extracellular target binding domain. In some embodiments, the two or more (e.g., two) target antigen binding domains are sdAbs specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the liner is a GS linker, an α-helical linker, a glycine-alanine polymer linker, an alanine-serine polymer linker, or an IgG4-Fc linker. In some embodiments, the two or more target antigen binding domains are connected by a linker selected from the group consisting of SEQ ID NOs: 1-21, 67, and 68.

In some embodiments, the extracellular target binding domain comprises an sdAb specifically binding to BCMA, such as any of the anti-BCMA sdAbs disclosed in PCT/CN2016/094408 and PCT/CN2017/096938. In some embodiments, the anti-BCMA sdAb comprises CDR1, CDR2, and CDR3 of the amino acid sequence of any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the anti-BCMA sdAb comprises CDR1, CDR2, and CDR3 of the amino acid sequence of SEQ ID NO: 26. Thus in some embodiments, there is provided a chimeric receptor polypeptide comprising (or consisting essentially of or consisting of): a) an extracellular target binding domain comprising an anti-BCMA sdAb (e.g., comprising SEQ ID NO: 26); b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of): a) an extracellular target binding domain comprising an anti-BCMA sdAb (e.g., comprising SEQ ID NO: 26); b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of): a) an extracellular target binding domain comprising two or more (such as two) anti-BCMA sdAbs; b) an extracellular TCR binding domain comprising a TCR antigen binding domain (such as scFv or sdAb) specifically binding to CD3 (such as CD3ε, for example the N-terminus of CD3ε); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of): a) an extracellular target binding domain comprising two or more (such as two) anti-BCMA sdAbs; b) an extracellular TCR binding domain comprising a TCR antigen binding domain specifically binding to CD3ε, wherein the TCR antigen binding domain is an sdAb comprising the amino acid sequence of SEQ ID NO: 22 or 23, or is an scFv comprising the amino acid sequence of SEQ ID NO: 24; c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) CD3ε extracellular domain without CD3ε signal peptide; g) a transmembrane domain comprising a transmembrane domain of CD3ε; and h) an intracellular domain comprising an intracellular domain of CD3ε. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) CD3γ extracellular domain without CD3γ signal peptide; g) a transmembrane domain comprising a transmembrane domain of CD3γ; and h) an intracellular domain comprising an intracellular domain of CD3γ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) CD3δ extracellular domain without CD3δ signal peptide; g) a transmembrane domain comprising a transmembrane domain of CD3δ; and h) an intracellular domain comprising an intracellular domain of CD3δ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) an optional hinge region (e.g., CD8 hinge); g) a transmembrane domain comprising a transmembrane domain of CD3ε; and h) an intracellular domain comprising an intracellular domain of CD3ε. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) an optional hinge region (e.g., CD8 hinge); g) a transmembrane domain comprising a transmembrane domain of CD3γ; and h) an intracellular domain comprising an intracellular domain of CD3γ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) an optional hinge region (e.g., CD8 hinge); g) a transmembrane domain comprising a transmembrane domain of CD3δ; and h) an intracellular domain comprising an intracellular domain of CD3δ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) CD3ε extracellular domain without CD3ε signal peptide; g) a transmembrane domain comprising a transmembrane domain of CD3ε; and h) an intracellular domain comprising an intracellular domain of CD3ε. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) CD3γ extracellular domain without CD3γ signal peptide; g) a transmembrane domain comprising a transmembrane domain of CD3γ; and h) an intracellular domain comprising an intracellular domain of CD3γ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) CD3δ extracellular domain without CD3δ signal peptide; g) a transmembrane domain comprising a transmembrane domain of CD3δ; and h) an intracellular domain comprising an intracellular domain of CD3δ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) an optional hinge region (e.g., CD8 hinge); g) a transmembrane domain comprising a transmembrane domain of CD3ε; and h) an intracellular domain comprising an intracellular domain of CD3ε. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) an optional hinge region (e.g., CD8 hinge); g) a transmembrane domain comprising a transmembrane domain of CD3γ; and h) an intracellular domain comprising an intracellular domain of CD3γ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) an optional hinge region (e.g., CD8 hinge); g) a transmembrane domain comprising a transmembrane domain of CD3δ; and h) an intracellular domain comprising an intracellular domain of CD3δ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) constant region of TCRα; g) a transmembrane domain comprising a transmembrane domain of TCRα; and h) an intracellular domain comprising an intracellular domain of TCRα. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) constant region of TCRβ; g) a transmembrane domain comprising a transmembrane domain of TCRβ; and h) an intracellular domain comprising an intracellular domain of TCRβ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) constant region of TCRγ; g) a transmembrane domain comprising a transmembrane domain of TCRγ; and h) an intracellular domain comprising an intracellular domain of TCRγ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) constant region of TCRδ; g) a transmembrane domain comprising a transmembrane domain of TCRδ; and h) an intracellular domain comprising an intracellular domain of TCRδ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) a transmembrane domain comprising a transmembrane domain of TCRα; and g) an intracellular domain comprising an intracellular domain of TCRα. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) a transmembrane domain comprising a transmembrane domain of TCRβ; and g) an intracellular domain comprising an intracellular domain of TCRβ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) a transmembrane domain comprising a transmembrane domain of TCRγ; and g) an intracellular domain comprising an intracellular domain of TCRγ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-CD3 scFv (e.g., comprising SEQ ID NO: 24); e) an optional second linker (e.g., GS linker); f) a transmembrane domain comprising a transmembrane domain of TCRδ; and g) an intracellular domain comprising an intracellular domain of TCRδ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) constant region of TCRα; g) a transmembrane domain comprising a transmembrane domain of TCRα; and h) an intracellular domain comprising an intracellular domain of TCRα. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) constant region of TCRβ; g) a transmembrane domain comprising a transmembrane domain of TCRβ; and h) an intracellular domain comprising an intracellular domain of TCRβ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) constant region of TCRγ; g) a transmembrane domain comprising a transmembrane domain of TCRγ; and h) an intracellular domain comprising an intracellular domain of TCRγ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) constant region of TCRδ; g) a transmembrane domain comprising a transmembrane domain of TCRδ; and h) an intracellular domain comprising an intracellular domain of TCRδ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) a transmembrane domain comprising a transmembrane domain of TCRα; and g) an intracellular domain comprising an intracellular domain of TCRα. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) a transmembrane domain comprising a transmembrane domain of TCRβ; and g) an intracellular domain comprising an intracellular domain of TCRβ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) a transmembrane domain comprising a transmembrane domain of TCRγ; and g) an intracellular domain comprising an intracellular domain of TCRγ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-CD3 scFv (e.g., comprising SEQ ID NO: 24); e) an optional third linker (e.g., GS linker); f) a transmembrane domain comprising a transmembrane domain of TCRδ; and g) an intracellular domain comprising an intracellular domain of TCRδ. In some embodiments, the anti-CD3 scFv comprises the amino acid sequence of SEQ ID NO: 24. In some embodiments, the anti-CD3 scFv comprises HC-CDR 1-3 and LC-CDR 1-3 of the amino acid sequence of SEQ ID NO: 24. In some embodiments, the anti-BCMA sdAb is any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the extracellular TCR binding domain comprises two or more anti-CD3 scFvs arranged in tandem. In some embodiments, the first linker, second linker, and/or the third linker is a GS linker, an α-helical linker, a glycine-alanine polymer linker, an alanine-serine polymer linker, or an IgG4-Fc linker. In some embodiments, the first linker, second linker, and/or the third linker comprises the amino acid sequence of any of SEQ ID NOs: 1-21, 67, and 68. In some embodiments, there is provided a chimeric receptor polypeptide comprising (or consisting essentially of or consisting of) the amino acid sequence of any of SEQ ID NOs: 43-48. In some embodiments, there is provided a chimeric receptor polypeptide comprising (or consisting essentially of or consisting of) the amino acid sequence of any of SEQ ID NOs: 51-58. In some embodiments, there is provided a chimeric receptor polypeptide comprising (or consisting essentially of or consisting of) the amino acid sequence of SEQ ID NO: 61.

In some embodiments, there is provided a chimeric receptor polypeptide comprising (or consisting essentially of or consisting of): a) an extracellular target binding domain comprising an anti-BCMA sdAb (e.g., comprising SEQ ID NO: 26); b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of): a) an extracellular target binding domain comprising an anti-BCMA sdAb (e.g., comprising SEQ ID NO: 26); b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of): a) an extracellular target binding domain comprising two or more (such as two) anti-BCMA sdAbs; b) an extracellular TCR binding domain comprising a TCR antigen binding domain (such as scFv or sdAb) specifically binding to TCR (such as TCRγ/δ, for example the constant region of TCRγ/δ); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of): a) an extracellular target binding domain comprising two or more (such as two) anti-BCMA sdAbs; b) an extracellular TCR binding domain comprising a TCR antigen binding domain specifically binding to TCRγ/δ, wherein the TCR antigen binding domain is an scFv comprising the amino acid sequence of SEQ ID NO: 27; c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) CD3ε extracellular domain without CD3ε signal peptide; g) a transmembrane domain comprising a transmembrane domain of CD3ε; and h) an intracellular domain comprising an intracellular domain of CD3ε. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) CD3γ extracellular domain without CD3γ signal peptide; g) a transmembrane domain comprising a transmembrane domain of CD3γ; and h) an intracellular domain comprising an intracellular domain of CD3γ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) CD3δ extracellular domain without CD3δ signal peptide; g) a transmembrane domain comprising a transmembrane domain of CD3δ; and h) an intracellular domain comprising an intracellular domain of CD3δ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) an optional hinge region (e.g., CD8 hinge); g) a transmembrane domain comprising a transmembrane domain of CD3ε; and h) an intracellular domain comprising an intracellular domain of CD3ε. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) an optional hinge region (e.g., CD8 hinge); g) a transmembrane domain comprising a transmembrane domain of CD3γ; and h) an intracellular domain comprising an intracellular domain of CD3γ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) an optional hinge region (e.g., CD8 hinge); g) a transmembrane domain comprising a transmembrane domain of CD3δ; and h) an intracellular domain comprising an intracellular domain of CD3δ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) CD3ε extracellular domain without CD3ε signal peptide; g) a transmembrane domain comprising a transmembrane domain of CD3ε; and h) an intracellular domain comprising an intracellular domain of CD3ε. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) CD3γ extracellular domain without CD3γ signal peptide; g) a transmembrane domain comprising a transmembrane domain of CD3γ; and h) an intracellular domain comprising an intracellular domain of CD3γ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) CD3δ extracellular domain without CD3δ signal peptide; g) a transmembrane domain comprising a transmembrane domain of CD3δ; and h) an intracellular domain comprising an intracellular domain of CD3δ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) an optional hinge region (e.g., CD8 hinge); g) a transmembrane domain comprising a transmembrane domain of CD3ε; and h) an intracellular domain comprising an intracellular domain of CD3ε. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) an optional hinge region (e.g., CD8 hinge); g) a transmembrane domain comprising a transmembrane domain of CD3γ; and h) an intracellular domain comprising an intracellular domain of CD3γ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) an optional hinge region (e.g., CD8 hinge); g) a transmembrane domain comprising a transmembrane domain of CD3δ; and h) an intracellular domain comprising an intracellular domain of CD3δ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) constant region of TCRα; g) a transmembrane domain comprising a transmembrane domain of TCRα; and h) an intracellular domain comprising an intracellular domain of TCRα. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) constant region of TCRβ; g) a transmembrane domain comprising a transmembrane domain of TCRβ; and h) an intracellular domain comprising an intracellular domain of TCRβ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) constant region of TCRγ; g) a transmembrane domain comprising a transmembrane domain of TCRγ; and h) an intracellular domain comprising an intracellular domain of TCRγ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) constant region of TCRδ; g) a transmembrane domain comprising a transmembrane domain of TCRδ; and h) an intracellular domain comprising an intracellular domain of TCRδ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) a transmembrane domain comprising a transmembrane domain of TCRα; and g) an intracellular domain comprising an intracellular domain of TCRα. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) a transmembrane domain comprising a transmembrane domain of TCRβ; and g) an intracellular domain comprising an intracellular domain of TCRβ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) a transmembrane domain comprising a transmembrane domain of TCRγ; and g) an intracellular domain comprising an intracellular domain of TCRγ. In some embodiments, there is provided a chimeric receptor polypeptide comprising (or consisting essentially of or consisting of), from N′ to C′: a) an optional signal peptide; b) an extracellular target binding domain comprising an anti-BCMA sdAb (e.g., comprising SEQ ID NO: 26); c) an optional first linker (e.g., GS linker); d) an extracellular TCR binding domain comprising an anti-TCR scFv (e.g., comprising SEQ ID NO: 27); e) an optional second linker (e.g., GS linker); f) a transmembrane domain comprising a transmembrane domain of TCRδ; and g) an intracellular domain comprising an intracellular domain of TCRδ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) constant region of TCRα; g) a transmembrane domain comprising a transmembrane domain of TCRα; and h) an intracellular domain comprising an intracellular domain of TCRα. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) constant region of TCRβ; g) a transmembrane domain comprising a transmembrane domain of TCRβ; and h) an intracellular domain comprising an intracellular domain of TCRβ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) constant region of TCRγ; g) a transmembrane domain comprising a transmembrane domain of TCRγ; and h) an intracellular domain comprising an intracellular domain of TCRγ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) constant region of TCRδ; g) a transmembrane domain comprising a transmembrane domain of TCRδ; and h) an intracellular domain comprising an intracellular domain of TCRδ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) a transmembrane domain comprising a transmembrane domain of TCRα; and g) an intracellular domain comprising an intracellular domain of TCRα. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) a transmembrane domain comprising a transmembrane domain of TCRβ; and g) an intracellular domain comprising an intracellular domain of TCRβ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) a transmembrane domain comprising a transmembrane domain of TCRγ; and g) an intracellular domain comprising an intracellular domain of TCRγ. In some embodiments, there is provided a chimeric receptor polypeptide comprising (or consisting essentially of or consisting of), from N′ to C′: a) an optional signal peptide; b) an extracellular target binding domain comprising a first anti-BCMA sdAb (e.g., comprising SEQ ID NO: 26) and a second anti-BCMA sdAb (e.g., comprising SEQ ID NO: 26) arranged in tandem, optionally connected by an optional first linker (e.g., GS linker); c) an optional second linker (e.g., GS linker); d) an extracellular TCR binding domain comprising an anti-TCR scFv (e.g., comprising SEQ ID NO: 27); e) an optional third linker (e.g., GS linker); f) a transmembrane domain comprising a transmembrane domain of TCRδ; and g) an intracellular domain comprising an intracellular domain of TCRδ. In some embodiments, the anti-TCR scFv comprises the amino acid sequence of SEQ ID NO: 27. In some embodiments, the anti-TCR scFv comprises HC-CDR 1-3 and LC-CDR 1-3 of the amino acid sequence of SEQ ID NO: 27. In some embodiments, the anti-BCMA sdAb is any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the extracellular TCR binding domain comprises two or more anti-TCR scFvs arranged in tandem. In some embodiments, the first linker, second linker, and/or the third linker is a GS linker, an α-helical linker, a glycine-alanine polymer linker, an alanine-serine polymer linker, or an IgG4-Fc linker. In some embodiments, the first linker, second linker, and/or the third linker comprises the amino acid sequence of any of SEQ ID NOs: 1-21, 67, and 68. In some embodiments, there is provided a chimeric receptor polypeptide comprising (or consisting essentially of or consisting of) the amino acid sequence of SEQ ID NO: 62 or 63.

The different aspects are discussed in various sections below in further detail.

### Extracellular Target Binding Domain

The extracellular target binding domain specifically binds to a target molecule.

The extracellular target binding domain in some embodiments comprises two or more (such as two) target antigen binding domains (e.g., scFv, sdAb, DARPin). In some embodiments, the two or more (such as two) target antigen binding domains (e.g., scFv, sdAb, DARPin) specifically bind to the same target molecules. For example, in some embodiments the extracellular target binding domain comprises two or more (such as two) target antigen binding domains (e.g., scFv, sdAb, DARPin), each recognizing the same or a different epitope on the same target antigen (e.g., BCMA). In some embodiments, the extracellular target binding domain comprises two or more (such as two) target antigen binding domains (e.g., scFv, sdAb, DARPin). each recognizing a different target antigen. In some embodiments, the extracellular target binding domain comprises two or more (such as two) VHH domains or sdAbs, each recognizing the same or a different epitope on a same target antigen (e.g., BCMA). In some embodiments, the extracellular target binding domain comprises two or more (such as two) VHH domains or sdAbs, each recognizing a different target antigen. In some embodiments, the two or more target antigen binding domains (e.g., scFv, sdAb, DARPin) are arranged in tandem. In some embodiments, the two or more target antigen binding domains (e.g., scFv, sdAb, DARPin) are connected by one or more linkers (such as any linker described herein). In some embodiments, the one or more linkers are selected from any of SEQ ID NOs: 1-21, 67, and 68. In some embodiments, the two or more target antigen binding domains (e.g., scFv, sdAb, DARPin) are the same. the two or more target antigen binding domains (e.g., scFv, sdAb, DARPin) are different.

In some embodiments, the target antigen binding domain comprises a ligand that specifically binds to a cognate receptor. Suitable ligand/receptor pairs include, but are not limited to, NGK2D ligand/receptor pair, IL2 ligand/receptor pair, BCMA ligand/receptor pair, TACI ligand/receptor pair, IL-13 ligand/receptor pair, IL-3 ligand/receptor pair, IL-4 ligand/receptor pair, VEGF ligand/receptor pair, HER1 ligand/receptor pair, HER2 ligand/receptor pair, and the like.

In some embodiments, the target antigen binding domain is an antigen binding domain that specifically binds to a target antigen (e.g., BCMA). Suitable target antigen binding domains include, but are not limited to, antibody or fragment thereof, such as single chain Fv (scFv), single domain antibody (sdAb), a VH, a VL, an scFv-scFv, an Fv, a Fab, a Fab′, a (Fab′)₂, a minibody, a diabody, a domain antibody variant (dAb), a V_{H}H, a fibronectin 3 domain variant, an ankyrin repeat variant such as a Designed Ankyrin Repeat Protein (“DARPin”), and other antigen-specific binding domains derived from other protein scaffolds. In some embodiments, the target antigen binding domain is an sdAb. In some embodiments, the target antigen binding domain is an scFv. In some embodiments, the target antigen binding domain is a DARPin. In some embodiments, reference to a target antigen binding domain that specifically binds to a target antigen means that the target antigen binding domain binds to the target antigen with a) an affinity that is at least about 10 (including for example at least about any of 10, 20, 30, 40, 50, 75, 100, 200, 300, 400, 500, 750, 1000 or more) times its binding affinity for other molecules; or b) a K_{d} no more than about ⅒ (such as no more than about any of ⅒, 1/20, 1/30, 1/40, 1/50, 1/75, 1/100, 1/200, 1/300, 1/400, 1/500, 1/750, 1/1000 or less) of the K_{d} for binding to other molecules. Binding affinity can be determined by methods known in the art, such as ELISA, fluorescence activated cell sorting (FACS) analysis, or radioimmunoprecipitation assay (RIA). K_{d} can be determined by methods known in the art, such as surface plasmon resonance (SPR) assay utilizing, for example, Biacore instruments, or kinetic exclusion assay (KinExA) utilizing, for example, Sapidyne instruments.

In some embodiments, the target molecule (such as target antigen) is a cell surface molecule (such as a cell surface antigen). In some embodiments, the cell surface molecule or antigen is selected from the group consisting of a protein, a carbohydrate, and a lipid. For example, tumor antigen can be a tumor-associated carbohydrate antigen (TACA), which is aberrant carbohydrate structure displayed on cancer cells that can be distinguished from normal cells. In some embodiments, the cell surface molecule or antigen is a disease-associated molecule or antigen expressed in a diseased cell. In some embodiments, the disease is cancer and the disease-associated molecule or antigen is a tumor-associated molecule or antigen expressed in a cancer cell. In some embodiments, the tumor-associated molecule or antigen is an oncoprotein. In some embodiments, the oncoprotein is the result of a mutation in a proto-oncogene, and the oncoprotein comprises a neoepitope comprising the mutation. For example, in some embodiments, the target molecule or antigen is a cell surface tumor-associated antigen (e.g., an oncoprotein comprising a neoepitope). In some embodiments, the disease is viral infection and the disease-associated molecule or antigen is a virus-associated molecule or antigen expressed in an infected cell. For example, in some embodiments, the target molecule or antigen is a cell surface virus-associated molecule or antigen. In some embodiments, the chimeric receptor polypeptide binds the target molecule or antigen with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values).

In some embodiments, the target molecule or antigen is selected from the group consisting of: tumor associated molecule, immune system associated molecule, virus infection associated molecule, and microbial proteins.

In some embodiments, the target molecule (such as target antigen) is a tumor associated antigen (TAA), such as a tumor associated antigen selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, bcr-abl p190 (e1a2), bcr-abl p210 (b2a2), bcr-abl p210 (b3a2), BING-4, CA-125, CAG-3, CAIX, CAMEL, Caspase-8, CD171, CD19, CD20, CD22, CD23, CD24, CD30, CD33, CD38, CD44v⅞, CD70, CD123, CD133, CDC27, CDK-4, CEA, CLCA2, CLL-1, CTAG1B, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, Glypican-3 (GPC3), GPC-2, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, L1-CAM, LILRB4, IGF1R, CCR5, CCR4, CXCR4, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1 /Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, a-folate receptor, and _{K}-light chain. In some embodiments, a first epitope and/or a second epitope can be AFP, EGFR, EGFRvIII, GPC3, GPC-2, DLL3, BCMA, CD19, CD20, CD22, CD123, CLL-1, CD30, CD33, HER2, MSLN, PSMA, CEA, GD2, IL,13Rα2, CAIX, L1-CAM, CA125, CD133, FAP, CTAG1B, MUC1, FR-α, CD70, CD171, ROR1, and any combination thereof. In some embodiments, the TAA is selected from the group consisting of BCMA, NY-ESO-1, VEGFR2, MAGE-A3, AFP, CD19, CD20, CD22, CD30, CD33, CD38, CD70, CD123, CEA, EGFR (such as EGFRvIII), GD2, GPC-2, GPC3, HER2, LILRB4, II,-13Rα2, IGF1R, mesothelin, PSMA, ROR1, WT1, NKG2D, CLL1, TGFaRII, TGFbRII, CCR5, CXCR4, and CCR4. In some embodiments, the TAA is BCMA.

In some embodiments, the target antigen binding domain is an sdAb specifically binding to BCMA, such as any of the anti-BCMA sdAbs disclosed in PCT/CN2016/094408 and PCT/CN2017/096938, the contents of which are incorporated herein by reference in their entirety. In some embodiments, the target antigen binding domain is an sdAb specifically binding to BCMA, comprising CDR1, CDR2, and CDR3 of the anti-BCMA sdAbs disclosed in PCT/CN2016/094408 and PCT/CN2017/096938. In some embodiments, the target antigen binding domain is an anti-BCMA sdAb comprising the amino acid sequence of SEQ ID NO: 26. In some embodiments, the target antigen binding domain is an anti-BCMA sdAb comprising CD1, CD2, and CD3 of the amino acid sequence of SEQ ID NO: 26. In some embodiments, the extracellular target binding domain comprises two or more (such as two) anti-BCMA sdAbs (e.g., arranged in tandem), such as any of the anti-BCMA sdAbs disclosed in PCT/CN2016/094408 and PCT/CN2017/096938. In some embodiments, the extracellular target binding domain comprises two or more (such as two) anti-BCMA sdAbs (e.g., arranged in tandem), each comprising the amino acid sequence of SEQ ID NO: 26.

In some embodiments, the target antigen is a neoantigen or neoepitope, such as a neoantigen or neoepitope encoded by a mutated gene. The gene can be selected from the group consisting of: ABL1, ACO1 1997, ACVR2A, AFP, AKT1, ALK, ALPPL2, ANAPC1, APC, ARID1A, AR, AR-v7, ASCL2, β2M, BRAF, BTK, C150RF40, CDH1, CLDN6, CNOT1, CT45A5, CTAG1B, DCT, DKK4,EEF1B2, EEF1DP3, EGFR, EIF2B3, env, EPHB2, ERBB3, ESR1, ESRP1, FAM11 IB, FGFR3, FRG1B,GAGE1, GAGE 10, GATA3, GBP3, HER2, IDH1, JAK1, KIT, KRAS, LMAN1, MABEB 16, MAGEA1,MAGEA10, MAGEA4, MAGEA8, MAGEB 17, MAGEB4, MAGEC1, MEK, MLANA, MLL2, MMP13,MSH3, MSH6, MYC, NDUFC2, NRAS, NY-ESO, PAGE2, PAGE5, PDGFRa, PIK3CA, PMEL, pol protein, POLE,PTEN, RAC1, RBM27, RNF43, RPL22, RUNX1, SEC31A, SEC63, SF3B 1, SLC35F5, SLC45A2, SMAP1, SMAP1, SPOP, TFAM, TGFBR2, THAP5, TP53, TTK, TYR, UBR5, VHL, and XPOT.

In some embodiments, the target antigen is a pathogen antigen, such as a fungal, viral, or bacterial antigen.

In some embodiments, the target antigen is a fungal antigen from Aspergillus or Candida, Cryplococcus , Histoplasma, Pneumocystis, or Stachybotrys.

In some embodiments, the target antigen is a virus-associated molecule or antigen (e.g., viral antigen) expressed in an infected cell. Some human cancers are caused by viruses, such as hepatitis B virus (liver cancer), papillomaviruses (cervical and other anogenital cancers; e.g., HPV), Epstein-Barr virus (EBV; Burkitt’s lymphoma and nasopharyngeal carcinoma), Kaposi’s sarcoma-associated herpesvirus (Kaposi’s sarcoma), and human T-cell lymphotropic virus (adult T-cell leukemia). Cancers that develop in AIDS patients are indirectly caused by HIV, as a result of immunodeficiency. In some embodiments, the target antigen is HIV related antigen, such as CCR5, CD4. Hepatitis C virus (an RNA virus) is an indirect cause of liver cancers resulting from chronic tissue damage. In some embodiments, the target antigen is a viral antigen from an oncogenic virus. Exemplary oncogenic viruses include, but are not limited to, EBV: EBNA-1, LMP-1, LMP-2A; HPV: E6, E7, E5; HBV: HBx; HCV: Core, NS3, NsSA; HTLV: Tax, HBZ; KSHV: vFLIP, LANA, vGPCR, vIRF-1. In some embodiments, the target antigen is a viral oncoprotein including, but not limited to, Tax, E7, E6/E7, E6, HBx, EBNA proteins (e.g., EBNA3 A, EBNA3 C, and EBNA 2), v-cyclin, LANA1, LANA2, LMP-1, k-bZIP, RTA, KSHV K8, and fragments thereof. Exemplary viral pathogens include those of the families of Adenoviridae, Epstein-Barr virus (EBV), Cytomegalovirus (CMV), Respiratory Syncytial Virus (RSV), JC virus, BK virus, HSV, HHV family of viruses, Picornaviridae, Herpesviridae, Hepadnaviridae, Flaviviridae, Retroviridae, Orthomyxoviridae, Parainyxoviridae, Papovaviridae, Polyomavirus, Rhabdoviridae, and Togavkidae. Exemplary pathogenic viruses cause smallpox, influenza, mumps, measles, chickenpox, ebola, and rubella. Target antigen can derive from any of the above-mentioned viruses. In some embodiments, the target antigen is a virus antigen of an opportunistic viral pathogen selected from the group consisting of CMV, adenovirus, BK virus, Human Herpes Virus-6 (HHV6) or other herpes viruses, influenza, respiratory syncytial virus (RSV), parainfluenza virus, and Varicella Zoster virus, HSV (Herpes simplex virus), EBV, JC virus, or Ebola.

In some embodiments, the target antigen is a bacterial antigen, such as Borrelia afzelii antigen, Borrelia garinii antigen, Brucella abortus antigen, Campylobacter jejuni antigen, Helicobacter pylori antigen, Legionella pneumophila antigen, Leptospira biflexa antigen, Mycoplasma pneumoniae antigen. Exemplary pathogenic bacteria include Streptococcus, Pseudomonas, Shigella, Campylobacter, Staphylococcus, Helicobacter, E, coli, Rickettsia, Bacillus, Bordetella, Chlamydia, Spirochetes, and Salmonella.

In some embodiments, the target antigen is an immune system associated molecule involved in tumor progression and/or escape from immune system surveillance, such as the immune system associated molecule on T cells, B cells, NK cells, macrophages, monocytes, etc. In some embodiments, the target antigen is CD4, CD8, CD45R, HLA-DR, immune checkpoint molecules, including but not limited to PD-L1 (B7-H1, CD274), PD-L2 (B7DC, CD273), B7-1 (CD80), B7-2 (CD86), Galectin9, HVEM, B7-H3 (CD276), FGL1, CD155, CD112, CD113, Galectin-9, CEACAM-1, and B7-H4.

In some embodiments, the target antigen is an autoimmune antigen. “Autoimmune antigen” refers to any self-protein or self-component that serves either as a target or causes of an autoimmune disease. Examples of autoimmune antigens include, but are not limited to, myelin basic protein, proteolipid protein, or myelin oligodendrocyte protein (multiple sclerosis); peripheral myelin proteins P0 and P2 (Guillain-Barre syndrome); acetylcholine receptor (myasthenia gravis); cardiac myosin (rheumatic fever/myocarditis); proteins of the beta cells in the Isles of Langerhans-GAD (glutamic acid decarboxylase), insulin (Type I autoimmune diabetes mellitus), the thyroid-stimulating hormone receptor (Grave’s disease), platelets (thrombocytopenic purpura), neuromuscular junction (myasthenia gravis), red blood cells (autoimmune hemolytic anemia and intracellular antigens (spliceosomes, ribosomes, nucleic acid, etc in systemic lupus erythematosus).

### Extracellular TCR Binding Domain

The extracellular TCR binding domain described herein specifically binds to one or more subunits (e.g., the extracellular domain of the subunits) in a TCR complex. The TCR complex is a complex of TCR subunits present on the surface of T cells composed of a TCR heterodimer (TCRα/TCRβ or TCRγ/TCRδ) and multiple CD3 subunits, namely, CD3ε, CD3δ, CD3γ, and CD3ζ. It participates in the activation of T cells in response to the binding of an antigen.

In some embodiments, the extracellular TCR binding domain comprises a TCR antigen binding domain (e.g., scFv, sdAb) specifically recognizing a TCR subunit (such as the extracellular domain of the TCR subunit), including for example any of TCRα, TCRβ, TCRγ, TCRδ, CD3ε, CD3δ, CD3y, and CD3ζ. In some embodiments, the TCR subunit specifically recognized by the TCR antigen binding domain (e.g., scFv, sdAb) is the same as the first TCR subunit (where transmembrane domain of the chimeric receptor peptide is derived from) and/or the second TCR subunit (where intracellular domain of the chimeric receptor peptide is derived from) described herein. In some embodiments, the TCR subunit specifically recognized by the TCR antigen binding domain (e.g., scFv, sdAb) is different from the first TCR subunit and/or the second TCR subunit described herein. In some embodiments, the TCR subunit specifically recognized by the TCR antigen binding domain (e.g., scFv, sdAb), the first TCR subunit, and the second TCR subunit described herein are all the same. In some embodiments, the first TCR subunit and the second TCR subunit described herein are the same, but different from the TCR subunit specifically recognized by the TCR antigen binding domain (e.g., scFv, sdAb).

In some embodiments, the TCR antigen binding domain comprises an antibody or antigen binding fragment thereof that binds to a subunit of CD3 (e.g., extracellular domain of CD3), such as CD3ε, e.g., N-terminus of CD3ε, CD3ε/γ, CD3₈/δ. Examples of CD3 antibodies are known in the art (for example muromonab, otelixizumab, and visilizumab). In some embodiments, the antibody or antigen binding fragment thereof that binds to a subunit of CD3 (such as CD3ε, e.g., N-terminus of CD3ε, CD3ε/γ, CD3ε/δ) is a single chain antibody, such as an sdAb or scFv. In some embodiments, the antibody or antigen binding fragment thereof that binds to a subunit of CD3 is derived from an anti-CD3 antibody, including but not limited to OKT3, UCHT1, TRX4, HuM291, SK7, and sdAbs disclosed in CN106084046A and CN106084047A, the contents of which are incorporated herein by reference in their entirety. In some embodiments, the TCR antigen binding domain (such as scFv) specifically binding to CD3 (e.g., extracellular domain of CD3ε) comprises HC-CDR1, HC-CDR2, HC-CDR3, LC-CDR1, LC-CDR2, and LC-CDR3 of UCHT1 anti-CD3 antibody (SEQ ID NO: 24). In some embodiments, the TCR antigen binding domain (such as scFv) specifically binding to CD3 (e.g., extracellular domain of CD3ε, CD3ε/γ, CD3ε/δ ) comprises VH and VL of UCHT1 anti-CD3 antibody (SEQ ID NO: 24). In some embodiments, the TCR antigen binding domain is an scFv specifically binding to CD3ε (e.g., extracellular domain of CD3ε), wherein the anti-CD3ε scFv comprises the amino acid sequence of SEQ ID NO: 24. In some embodiments, the TCR antigen binding domain is an sdAb specifically binding to CD3ε (e.g., extracellular domain of CD3ε). In some embodiments, the TCR antigen binding domain is an sdAb comprising CDR1, CDR2, and CDR3 of an anti-CD3 sdAb comprising the amino acid sequence of SEQ ID NO: 22 or 23. In some embodiments, the TCR antigen binding domain is an anti-CD3ε sdAb comprising the amino acid sequence of SEQ ID NO: 22 or 23.

In some embodiments, the TCR antigen binding domain comprises an antibody or antigen binding fragment thereof that binds to a TCR (e.g., extracellular domain of TCR), such as any of TCRα, TCRβ, TCRγ, or TCRδ, e.g., constant region of TCRγ/δ. Examples of TCR antibodies are known in the art (for example B1.1, IP26, H57-597, 1B2, WT31, 7F18, 3C10,

KJ1298). In some embodiments, the antibody or antigen binding fragment thereof that binds to TCR (such as TCRγ/δ, e.g., constant region of TCRγ/δ) is a single chain antibody, such as an sdAb or scFv. In some embodiments, the antibody or antigen binding fragment thereof that binds to TCR is derived from an anti-TCR antibody, including but not limited to B1.1, IP26, H57-597, 1B2, WT31, 7F18, 3C10, KJ1298. In some embodiments, the TCR antigen binding domain (such as scFv) specifically binding to TCR (e.g., TCRγ/δ) comprises HC-CDR1, HC-CDR2, HC-CDR3, LC-CDR1, LC-CDR2, and LC-CDR3 of B1.1 anti-TCRγ/δ antibody (SEQ ID NO: 27). In some embodiments, the TCR antigen binding domain (such as scFv) specifically binding to TCR (e.g., TCRγ/δ) comprises VH and VL of B1.1 anti-TCRγ/δ antibody (SEQ ID NO: 27). In some embodiments, the TCR antigen binding domain is an scFv specifically binding to TCRγ/δ (e.g., constant region of TCRγ/δ), wherein the anti-TCRγ/δ scFv comprises the amino acid sequence of SEQ ID NO: 27.

In some embodiments, the extracellular TCR binding domain binds to a TCR subunit (such as extracellular domain of a TCR subunit) with a) an affinity that is at least about 10 (including for example at least about any of 10, 20, 30, 40, 50, 75, 100, 200, 300, 400, 500, 750, 1000 or more) times its binding affinity for other molecules; or b) a Kd no more than about ⅒ (such as no more than about any of ⅒, 1/20, 1/30, 1/40, 1/50, 1/75, 1/100, 1/200, 1/300, 1/400, 1/500, 1/750, 1/1000 or less) of the Kd for binding to other molecules. Binding affinity can be determined by methods known in the art, such as ELISA, fluorescence activated cell sorting (FACS) analysis, or radioimmunoprecipitation assay (RIA). Kd can be determined by methods known in the art, such as surface plasmon resonance (SPR) assay utilizing, for example, Biacore instruments, or kinetic exclusion assay (KinExA) utilizing, for example, Sapidyne instruments.

The extracellular TCR binding domain in some embodiments comprises two or more (such as two) TCR antigen binding domains (e.g., scFv, sdAb). In some embodiments, the two or more TCR antigen binding domains are linked in tandem. In some embodiments, the two or more TCR antigen binding domains are connected by one or more linkers (such as any linkers described herein). In some embodiments, the one or more linker is selected from any of SEQ ID NOs: 1-21, 67, and 68. In some embodiments, the two or more (such as two) TCR antigen binding domains (e.g., scFv, sdAb) specifically bind to the same TCR subunit (e.g., extracellular domain of the same TCR subunit). For example, in some embodiments, the extracellular TCR binding domain comprises two or more (such as two) TCR antigen binding domains (e.g., scFv, sdAb), each recognizing the same or a different epitope on the same TCR subunit (e.g., epitope on the extracellular domain of the same TCR subunit). In some embodiments, the extracellular TCR binding domain comprises two or more (such as two) TCR antigen binding domains (e.g., scFv, sdAb), each recognizing a different TCR subunit (e.g., extracellular domain of different TCR subunits). In some embodiments, the two or more TCR antigen binding domains are the same. In some embodiments, the two or more TCR antigen binding domains are different.

In some embodiments, the extracellular TCR binding domain binds the TCR subunit (e.g., extracellular domain of the TCR subunit) with a Kd between about 0.1 pM to about 5 µM (such as about any of 0.1 pM, 1.0 pM, 10 pM, 50 pM, 100 pM, 500 pM, 1 nM, 10 nM, 50 nM, 100 nM, 500 nM, 1 µM, 2 µM, 3 µM, 4 µM, 5 µM, including any ranges between these values).

### Transmembrane Domain

The chimeric receptor polypeptides in some embodiments comprise a transmembrane domain. In some embodiments, the transmembrane domain comprises a transmembrane domain of a TCR subunit, such as any of CD3ε, CD3γ, CD3δ, TCRα, TCRβ, TCRγ, and TCRδ. In some embodiments, the transmembrane domain further comprises one or more (for example up to about 5) additional amino acids adj acent to the TCR subunit transmembrane domain in the extracellular and/or intracellular region.

The transmembrane domain of the chimeric receptor polypeptide may be derived either from a natural or from a synthetic source. As used herein, a “transmembrane domain” refers to any protein structure that is thermodynamically stable in a cell membrane, preferably a eukaryotic cell membrane. Transmembrane domains compatible for use in the chimeric receptor polypeptides described herein may be obtained from a naturally occurring protein, e.g., naturally occurring TCR subunit. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane- spanning segments and may be further sub-classified based on the number of transmembrane segments and the location of N- and C-termini.

In some embodiments, the transmembrane domain of the chimeric receptor polypeptide described herein is derived from a Type I single-pass membrane protein. In some embodiments, transmembrane domains from multi-pass membrane proteins may also be compatible for use in the chimeric receptor polypeptides described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In some embodiments when the chimeric receptor polypeptide comprises both the transmembrane domain of a TCR subunit and the intracellular domain of a TCR subunit, the transmembrane domain and the intracellular domain in the chimeric receptor polypeptide can be derived from the same TCR subunit or from different TCR subunits.

In some embodiments when the chimeric receptor polypeptide comprises both a transmembrane domain and an intracellular domain, and the intracellular domain comprises the intracellular domain of a TCR subunit, the transmembrane domain can be derived from the transmembrane of a protein that is not a TCR subunit, including for example CD28, CD45, CD4, CD5, CD8, CD9, CD16, CD22, CD27, CD33, CD37, CD40, CD40L, CD45, CD64, CD70, CD80, CD86, CD95, CD134, CD137 (4-1BB), CD154, CD278 (ICOS), KIRDS2, OX40, CD2, LFA-1 (CD11a, CD18), GITR, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD 11d, ITGAE, CD103, ITGAL, ITGAM, CD11b, ITGAX, CD11c, ITGB1, CD29, ITGB2, CD18, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some embodiments, the transmembrane domain comprises a transmembrane domain of a TCR subunit selected from the group consisting of CD3ε, CD3γ, CD3δ, TCRα, TCRβ, TCRγ, and TCRδ. In some embodiments, the chimeric receptor polypeptide further comprises an extracellular domain of a TCR subunit selected from the group consisting of CD3ε, CD3γ, CD3δ, TCRα, TCRβ, TCRγ, and TCRδ, adjacent to the transmembrane domain of the chimeric receptor polypeptide in the extracellular region. In some embodiments, such extracellular domain does not comprise a signal peptide. In some embodiments, the TCR subunit from which the extracellular domain is derived is the same as the TCR subunit from which the transmembrane domain is derived. In some embodiments, the TCR subunit from which the extracellular domain is derived is different from the TCR subunit from which the transmembrane domain is derived. In some embodiments, the TCR subunit from which the extracellular domain is derived is the same as the TCR subunit from which the intracellular domain is derived. In some embodiments, the TCR subunit from which the extracellular domain is derived is different from the TCR subunit from which the intracellular domain is derived. In some embodiments, the TCR subunit from which the extracellular domain is derived, the TCR subunit from which the transmembrane domain is derived, and the TCR subunit from which the intracellular domain is derived are all the same. In some embodiments, the TCR subunit from which the extracellular domain is derived, the TCR subunit from which the transmembrane domain is derived, and the TCR subunit from which the intracellular domain is derived are all different. In some embodiments, the TCR subunit from which the extracellular domain is derived is the same as the TCR subunit from which the transmembrane domain is derived, but different from the TCR subunit from which the intracellular domain is derived. In some embodiments, the TCR subunit from which the extracellular domain is derived is the same as the TCR subunit from which the intracellular domain is derived, but different from the TCR subunit from which the transmembrane domain is derived. In some embodiments, the TCR subunit from which the intracellular domain is derived is the same as the TCR subunit from which the transmembrane domain is derived, but different from the TCR subunit from which the extracellular domain is derived. For example, in some embodiments, the chimeric receptor polypeptide comprises the CD3ε extracellular domain (without signal peptide) adjacent to the CD3ε transmembrane domain in the extracellular region. In some embodiments, the chimeric receptor polypeptide comprises the CD3γ extracellular domain (without signal peptide) adjacent to the CD3γ transmembrane domain in the extracellular region. In some embodiments, the chimeric receptor polypeptide comprises the CD3δ extracellular domain (without signal peptide) adjacent to the CD3δ transmembrane domain in the extracellular region. In some embodiments, the chimeric receptor polypeptide comprises the TCRα extracellular domain (without signal peptide) adj acent to the TCRα transmembrane domain in the extracellular region. In some embodiments, the chimeric receptor polypeptide comprises the TCRβ extracellular domain (without signal peptide) adjacent to the TCRβ transmembrane domain in the extracellular region. In some embodiments, the chimeric receptor polypeptide comprises the TCRγ extracellular domain (without signal peptide) adjacent to the TCRγ transmembrane domain in the extracellular region. In some embodiments, the chimeric receptor polypeptide comprises the TCRδ extracellular domain (without signal peptide) adjacent to the TCRδ transmembrane domain in the extracellular region. In some embodiments, the TCR subunit from which the extracellular domain is derived is selected from the group of TCRα, TCRβ, TCRγ, and TCRδ, and the chimeric receptor polypeptide comprises only the constant region of said TCR subunit adj acent to the transmembrane domain of the chimeric receptor polypeptide in the extracellular region. For example, in some embodiments, the chimeric receptor polypeptide comprises the TCRα constant region (without TCRα variable region and signal peptide) adjacent to the TCRα transmembrane domain in the extracellular region. In some embodiments, the chimeric receptor polypeptide comprises the TCRβ constant region (without TCRβ variable region and signal peptide) adjacent to the TCRβ transmembrane domain in the extracellular region. In some embodiments, the chimeric receptor polypeptide comprises the TCRγ constant region (without TCRγ variable region and signal peptide) adj acent to the TCRγ transmembrane domain in the extracellular region. In some embodiments, the chimeric receptor polypeptide comprises the TCRδ constant region (without TCRδ variable region and signal peptide) adjacent to the TCRδ transmembrane domain in the extracellular region. In some embodiments, the chimeric receptor polypeptide does not comprise an extracellular domain of any TCR subunit. In some embodiments, the chimeric receptor polypeptide comprises CD3ε extracellular domain (without signal peptide), CD3ε transmembrane domain, and CD3ε intracellular domain, such as a full length CD3ε without CD3ε signal peptide (hereinafter also referred to as construct “e”). In some embodiments, the chimeric receptor polypeptide comprises CD3γ extracellular domain (without signal peptide), CD3γ transmembrane domain, and CD3γ intracellular domain, such as a full length CD3γ without CD3γ signal peptide (hereinafter also referred to as construct “g”). In some embodiments, the chimeric receptor polypeptide comprises CD3δ extracellular domain (without signal peptide), CD3δ transmembrane domain, and CD3δ intracellular domain, such as a full length CD3δ without CD3δ signal peptide (hereinafter also referred to as construct “d”). In some embodiments, the chimeric receptor polypeptide comprises CD3ε transmembrane domain and CD3ε intracellular domain without any CD3ε extracellular domain (hereinafter also referred to as construct “se”, SEQ ID NO: 40). In some embodiments, the chimeric receptor polypeptide comprises CD3γ transmembrane domain and CD3γ intracellular domain without any CD3γ extracellular domain (hereinafter also referred to as construct “sg”, SEQ ID NO: 41). In some embodiments, the chimeric receptor polypeptide comprises CD3δ transmembrane domain and CD3δ intracellular domain without any CD3δ extracellular domain (hereinafter also referred to as construct “sd”, SEQ ID NO: 42). In some embodiments, the chimeric receptor polypeptide comprises TCRα constant region (without TCRα variable region and signal peptide), TCRα transmembrane domain, and TCRα intracellular domain (hereinafter also referred to as construct “taC”, SEQ ID NO: 32). In some embodiments, the chimeric receptor polypeptide comprises TCRβ constant region (without TCRβ variable region and signal peptide), TCRβ transmembrane domain, and TCRβ intracellular domain (hereinafter also referred to as construct “tbC”, SEQ ID NO: 33). In some embodiments, the chimeric receptor polypeptide comprises TCRγ constant region (without TCRγ variable region and signal peptide), TCRγ transmembrane domain, and TCRγ intracellular domain (hereinafter also referred to as construct “tgC”, SEQ ID NO: 36). In some embodiments, the chimeric receptor polypeptide comprises TCRδ constant region (without TCRbvariable region and signal peptide), TCRδ transmembrane domain, and TCRδ intracellular domain (hereinafter also referred to as construct “tdC”, SEQ ID NO: 37). In some embodiments, the chimeric receptor polypeptide comprises TCRα transmembrane domain and TCRα intracellular domain without any TCRα extracellular domain (hereinafter also referred to as construct “sta”, SEQ ID NO: 34). In some embodiments, the chimeric receptor polypeptide comprises TCRβ transmembrane domain and TCRβ intracellular domain without any TCRβ extracellular domain (hereinafter also referred to as construct “stb”, SEQ ID NO: 35). In some embodiments, the chimeric receptor polypeptide comprises TCRγ transmembrane domain and TCRγ intracellular domain without any TCRγ extracellular domain (hereinafter also referred to as construct “stg”, SEQ ID NO: 38). In some embodiments, the chimeric receptor polypeptide comprises TCRδ transmembrane domain and TCRδ intracellular domain without any TCRδ extracellular domain (hereinafter also referred to as construct “std”, SEQ ID NO: 39.

### Hinge Region

In some embodiments, the transmembrane domain of the chimeric receptor polypeptide described herein comprises an extracellular hinge region (hereinafter also referred to as “STS hinge region”). In some embodiments, the STS hinge region comprises a hinge region of a TCR subunit or a fragment thereof, for example, a hinge region of the same TCR subunit (e.g., CD3ε) from which the transmembrane domain of the chimeric receptor polypeptide is derived from (e.g., see, “Transmembrane domain” subsection). In some embodiments, the hinge region is derived from a different TCR subunit (e.g., CD3γ) compared to where the transmembrane domain of the chimeric receptor polypeptide is derived from (e.g., CD3ε).

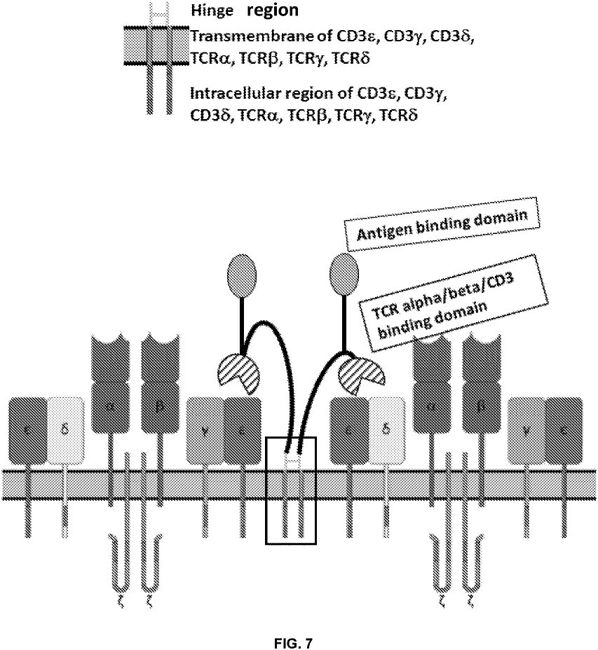

In some embodiments when the chimeric receptor polypeptide does not comprise an extracellular domain of an TCR subunit (e.g., CD3ε, CD3γ, CD3δ), a hinge region (e.g., CD8 hinge), and optionally an additional linker (such as GS linker) is used to connect the transmembrane domain of the chimeric receptor polypeptide and the extracellular TCR binding domain (when the extracellular target binding domain is N-terminal to the extracellular TCR binding domain) or the extracellular target binding domain (when the extracellular target binding domain is C-terminal to the extracellular TCR binding domain). In some embodiments, the hinge region facilitates the dimerization of two chimeric receptor polypeptides on the cell surface (see, e.g., FIGS. 7-9).

In some embodiments, the hinge region is a hinge region of a naturally occurring protein. Hinge regions of any protein known in the art to comprise a hinge region are compatible for use in the chimeric receptor polypeptides described herein. In some embodiments, the hinge region is at least a portion of a hinge region of a naturally occurring protein and confers flexibility to the chimeric receptor polypeptide. In some embodiments, the hinge region comprises a hinge region of CD8. In some embodiments, the hinge comprises the amino acid sequence of SEQ ID NO: 31.

Hinge regions of antibodies, such as an IgG, IgA, IgM, IgE, or IgD antibodies, are also compatible for use in the chimeric receptor polypeptides described herein. In some embodiments, the hinge region is the hinge region that joins the constant domains CH1 and CH2 of an antibody. In some embodiments, the hinge region is of an antibody and comprises the hinge region of the antibody and one or more constant regions of the antibody. In some embodiments, the hinge region comprises the hinge region of an antibody and the CH3 constant region of the antibody. In some embodiments, the hinge region comprises the hinge region of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge regions for the chimeric receptor polypeptides described herein.

The hinge region may contain about 5-100 amino acids, e.g., about any one of 5-15 amino acids, 15-75 amino acids, 20-50 amino acids, or 30-60 amino acids. In some embodiments, the hinge region may be at least about any one of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge region and the transmembrane domain of the chimeric receptor polypeptides described herein are derived from the same molecule (e.g., the same immune cell co-stimulator, immune cell co-inhibitor, TCR subunit, or T cell co-receptor). For example, in some embodiments, the hinge region of the chimeric receptor polypeptide comprises a TCRα hinge region or a fragment thereof, and the transmembrane domain of the chimeric receptor polypeptide comprises a TCRα transmembrane domain or a fragment thereof. In some embodiments, the hinge region and the transmembrane domain of the chimeric receptor polypeptides are derived from different molecules (e.g., different immune cell co-stimulator, immune cell co-inhibitor, TCR subunit, or T cell co-receptor). For example, in some embodiments, the hinge region of the chimeric receptor polypeptide comprises a 4-1BB hinge region or a fragment thereof, and the transmembrane domain of the chimeric receptor polypeptide comprises a CD3ε transmembrane domain or a fragment thereof.

### Intracellular Domains

The chimeric receptor polypeptides in some embodiments comprise an intracellular domain. In some embodiments, the intracellular domain comprises an intracellular domain of a TCR subunit, such as any of CD3ε, CD3γ, CD3δ, TCRα, TCRβ, TCRγ, and TCRδ. In some embodiments, the intracellular domain comprises a signaling domain (e.g., intracellular domains of CD3ε, CD3γ, and CD3δ). In some embodiments, the intracellular domain lacks a signaling domain (e.g., intracellular domains of TCRα, TCRβ, TCRγ, and TCRδ). In some embodiments, the intracellular domain lacks a co-stimulatory domain.

In some embodiments, the intracellular domain comprises a portion of or the entire intracellular domain of CD3ε. In some embodiments, the intracellular domain comprises a portion of or the entire intracellular domain of CD3γ. In some embodiments, the intracellular domain comprises a portion of or the entire intracellular domain of CD3δ.

In some embodiments, the intracellular domain comprises a portion of or the entire intracellular domain of TCRα. In some embodiments, the intracellular domain comprises a portion of or the entire intracellular domain of TCRβ. In some embodiments, the intracellular domain comprises a portion of or the entire intracellular domain of TCRγ. In some embodiments, the intracellular domain comprises a portion of or the entire intracellular domain of TCRδ.

In some embodiments, the intracellular domain does not comprise the immunoreceptor tyrosine-based activation motif (ITAM). An “ITAM,” as used herein, is a conserved protein motif that is generally present in the tail portion of signaling molecules expressed in many immune cells. The ITAM may comprise two repeats of the amino acid sequence YxxL/I separated by 6-8 amino acids, wherein each x is independently any amino acid, producing the conserved motif YxxL/Ix(6-8)YxxL/I. ITAMs within signaling molecules (e.g., CD3ζ, CD3ε, CD3γ, CD3δ) are important for signal transduction within the cell, which is mediated at least in part by phosphorylation of tyrosine residues in the ITAM (e.g., by the Src family kinases such as Lck) following activation of the signaling molecule. ITAMs may also function as docking sites for other proteins involved in signaling pathways. Exemplary ITAM-containing primary cytoplasmic signaling sequences include those derived from CD3ζ, FcR gamma (FCER1G), FcR beta (Fc Epsilon Rib), CD3γ, CD3δ, CD3ε, CD5, CD22, CD79a, CD79b, and CD66d. In some embodiments, the intracellular domain described herein is not derived from an ITAM-containing molecule. For example, in some embodiments, the intracellular domain does not comprise an intracellular domain of CD3ζ, CD3γ, CD3δ, CD3ε, or a fragment thereof. In some embodiments, the intracellular domain described herein is derived from an ITAM-containing molecule but without any ITAM sequence. For example, in some embodiments, the intracellular domain comprises an intracellular domain of CD3γ, CD3δ, or CD3ε, with ITAM sequence deleted. In some embodiments, the intracellular domain comprises a sequence of amino acids that can recruit and interact with the tyrosine kinase Lck.

In some embodiments, the TCR subunit from which the intracellular domain of the chimeric receptor polypeptide is derived is the same as the TCR subunit from which the transmembrane domain of the chimeric receptor polypeptide is derived. In some embodiments, the TCR subunit from which the intracellular domain of the chimeric receptor polypeptide is derived is different from the TCR subunit from which the transmembrane domain of the chimeric receptor polypeptide is derived.

### Linkers

The chimeric receptor polypeptide described herein in some embodiments can comprise a linker (e.g., peptide linker) between one or more domains described herein. For example, in some embodiments, the chimeric receptor polypeptide comprises a first linker connecting the extracellular target binding domain with the extracellular TCR binding domain. In some embodiments, the chimeric receptor polypeptide further comprises a second linker. When the extracellular target binding domain is at the N-terminus of the extracellular TCR binding domain, the C-terminus of the extracellular TCR binding domain and the N-terminus of the transmembrane domain (or the N-terminus of the extracellular hinge region if present at the N-terminus of the transmembrane domain) are connected by a second linker. When the extracellular target binding domain is at the C-terminus of the extracellular TCR binding domain, the C-terminus of the extracellular target binding domain and the N-terminus of the transmembrane domain (or the N-terminus of the extracellular hinge region if present at the N-terminus of the transmembrane domain) are connected by a second linker. In some embodiments, the two or more (such as two) target antigen binding domains (e.g., scFv, VHH, DARPin) comprised within the extracellular target binding domain are connected by a third linker. In some embodiments, the two or more (such as two) TCR antigen binding domains (e.g., scFv, sdAb, VHH) comprised within the extracellular TCR binding domain are connected by a fourth linker. In some embodiments, when the chimeric receptor polypeptide comprises an extracellular hinge region, the extracellular hinge region can also be linked to the N-terminus of the transmembrane domain via a linker. In some embodiments, there is a linker situated between the C-terminus of the transmembrane domain and the N-terminus of the intracellular domain of the chimeric receptor polypeptide. The linkers connecting one or more domains described herein can be the same or different, such as different in sequence and/or length.

The linkers can be peptide linkers of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the peptide linker is from about 1 amino acid to about 10 amino acids long, from about 2 amino acids to about 15 amino acids long, from about 3 amino acids to about 12 amino acids long, from about 4 amino acids to about 10 amino acids long, from about 5 amino acids to about 9 amino acids long, from about 6 amino acids to about 8 amino acids long, from about 1 amino acid to about 20 amino acids long, from about 10 amino acid to about 20 amino acids long, from about 21 amino acids to about 30 amino acids long, from about 10 amino acids to about 25 amino acids long, from about 5 amino acids to about 15 amino acids long, from about 15 amino acids to about 20 amino acids long, from about 1 amino acid to about 30 amino acids long, from about 5 amino acid to about 30 amino acids long, from about 2 amino acids to about 20 amino acids long, from about 5 amino acids to about 25 amino acids long, from about 5 amino acids to about 24 amino acids long, from about 6 amino acids to about 23 amino acids long, from about 5 amino acids to about 22 amino acids long, from about 6 amino acids to about 21 amino acids long, from about 7 amino acids to about 20 amino acids long, from about 10 amino acids to about 30 amino acids long, from about 30 amino acid to about 40 amino acids long, from about 40 amino acid to about 50 amino acids long, from about 30 amino acids to about 50 amino acids long, from about 50 amino acids to about 100 amino acids long, from about 1 amino acid to about 100 amino acids long, from about 2 amino acids to about 19 amino acids long, from about 3 amino acids to about 18 amino acids long, from about 4 amino acids to about 17 amino acids long, from about 4 amino acids to about 16 amino acids long, from about 4 amino acids to about 15 amino acids long, from about 4 amino acids to about 14 amino acids long, from about 4 amino acids to about 13 amino acids long, from about 4 amino acids to about 12 amino acids long, from about 4 amino acids to about 11 amino acids long, from about 4 amino acids to about 9 amino acids long, from about 4 amino acids to about 8 amino acids long, from about 4 amino acids to about 7 amino acids long, or from about 4 amino acids to about 6 amino acids long. In some embodiments, the peptide linker is any of 1, 2, 3, 4, 5, 6, 7, 8, 9, 10, 11, 12, 13, 14, 15, 16, 17, 18, 19 or 20 amino acids long. In some embodiments, the peptide linker is any of 21, 22, 23, 24, 25, 26, 27, 28, 29, or 30 amino acids long.

A peptide linker can have a naturally occurring sequence or a non-naturally occurring sequence. For example, a sequence derived from the hinge region of a heavy chain only antibody can be used as a linker. See, for example, WO1996/34103. In some embodiments, the peptide linker is a human IgG1 or IgG4 hinge. In some embodiments, the peptide linker is a mutated human IgG1 or IgG4 hinge. In some embodiments, the peptide linker is a IgG4-Fc-linker, such as an IgG4-Fc-linker comprising the amino acid sequence of SEQ ID NO: 21 (ESKYGPPCPPCPAPEFEGGPSVFLFPPKPKDTLMISRTPEVTCVVVDVSQEDPEVQFNWY VDGVEVHNAKTKPREEQFQSTYRVVSVLTVLHQDWLNGKEYKCKVSNKGLPSSIEKTIS KAKGQPREPQVYTLPPSQEEMTKNQVSLTCLVKGFYPSDIAVEWESNGQPENNYKTTPP VLDSDGSFFLYSRLTVDKSRWQEGNVFSCSVMHEALHNHYTQKSLSLSLGK). In some embodiments, the peptide linker is an α-helical linker. In some embodiments, the linker is a flexible linker. Exemplary flexible linkers include glycine polymers (G)ₙ (SEQ ID NO: 9), glycine-serine polymers (including, for example, (GS)ₙ (SEQ ID NO: 10), (GSGGS)ₙ (SEQ ID NO: 11), (GGGS)ₙ(SEQ ID NO: 12), or (GGGGS)ₙ (SEQ ID NO: 13), where n is an integer of at least one, such as 1, 2, 3, 4, 5), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). Exemplary flexible linkers include, but are not limited to Gly-Gly (SEQ ID NO: 14), Gly-Gly-Ser-Gly (SEQ ID NO: 15), Gly-Gly-Ser-Gly-Gly (SEQ ID NO: 16), Gly-Ser-Gly-Ser-Gly (SEQ ID NO: 17), Gly-Ser-Gly-Gly-Gly (SEQ ID NO: 18), Gly-Gly-Gly-Ser-Gly (SEQ ID NO: 19), Gly-Ser-Ser-Ser-Gly (SEQ ID NO: 20), Gly-Gly-Ser-Gly-Gly-Ser (SEQ ID NO: 5), Ser-Gly-Gly-Gly-Gly-Ser (SEQ ID NO: 6), Gly-Arg-Ala-Gly-Gly-Gly-Gly- Ala-Gly-Gly-Gly-Gly (SEQ ID NO: 7), Gly-Arg-Ala-Gly-Gly-Gly (SEQ ID NO: 8), GGGGSGGGGS GGGGSGGGGS (SEQ ID NO: 4), GGGGSGGGGSGGGGS (SEQ ID NO: 3), GGGGSGGGGS (SEQ ID NO: 2), GGGGS (SEQ ID NO: 1), GGGGGSGGGGSGGGGS (SEQ ID NO: 67), GGGGSS (SEQ ID NO: 68), and the like. The ordinarily skilled artisan will recognize that design of a chimeric receptor polypeptide can include linkers that are all or partially flexible, such that the linker can include a flexible linker portion as well as one or more portions that confer less flexible structure to provide a desired chimeric receptor polypeptide.

In some embodiments, the linker (e.g., peptide linker) is a stable linker (not cleavable by protease, especially matrix metalloproteinases (MMPs)).

In some embodiments, the linker (e.g., peptide linker) is a cleavable linker. In some embodiments, the linker comprises a protease substrate cleavage sequence, for example, an MMP substrate cleavage sequence. Substrate sequences that can be cleaved by MMPs have been extensively studied. For example, the sequence of PLGLAG (SEQ ID NO: 25) can be cleaved by most MMPs. In some embodiments, the protease cleavage site is recognized by MMP-2, MMP-9, or a combination thereof.

### Signal Peptide

The chimeric receptor polypeptide of the present application may comprise a signal peptide (also known as a signal sequence) at the N-terminus of the chimeric receptor polypeptide. In general, signal peptides are peptide sequences that target a polypeptide to the desired site in a cell. In some embodiments, the signal peptide targets the chimeric receptor polypeptide to the secretory pathway of the cell and will allow for integration and anchoring of the chimeric receptor polypeptide into the lipid bilayer. Signal peptides including signal sequences of naturally occurring proteins or synthetic, non-naturally occurring signal sequences, which are compatible for use in the chimeric receptor polypeptides (and/or cytokines described below) described herein will be evident to one of skill in the art. In some embodiments, the signal peptide is derived from a molecule selected from the group consisting of CD8α, GM-CSF receptor α, IL-3, and IgG1 heavy chain. In some embodiments, the signal peptide is derived from CD8α. In some embodiments, the signal peptide is derived from the same molecule from which the transmembrane domain and/or intracellular domain of the chimeric receptor polypeptide is derived (or same as the TCR subunit whose extracellular domain is present, adjacent to the transmembrane domain of the chimeric receptor polypeptide, e.g., CD3ε extracellular domain). For examples, in some embodiments, when the chimeric receptor polypeptide comprises a transmembrane domain derived from CD3ε, an intracellular domain derived from CD3ε, and/or an CD3ε extracellular domain, the signal peptide is also derived from CD3ε. In some embodiments, when the chimeric receptor polypeptide comprises a transmembrane domain derived from CD3γ, an intracellular domain derived from CD3γ, and/or an CD3γ extracellular domain, the signal peptide is also derived from CD3γ. In some embodiments, when the chimeric receptor polypeptide comprises a transmembrane domain derived from CD3δ, an intracellular domain derived from CD3δ, and/or an CD3δ extracellular domain, the signal peptide is also derived from CD3δ. In some embodiments, when the chimeric receptor polypeptide comprises a transmembrane domain derived from TCRα, an intracellular domain derived from TCRα, and/or an TCRα extracellular domain (e.g., TCRα constant region), the signal peptide is also derived from TCRα. In some embodiments, when the chimeric receptor polypeptide comprises a transmembrane domain derived from TCRβ, an intracellular domain derived from TCRβ, and/or an TCRβ extracellular domain (e.g., TCRβ constant region), the signal peptide is also derived from TCRβ. In some embodiments, when the chimeric receptor polypeptide comprises a transmembrane domain derived from TCRγ, an intracellular domain derived from TCRγ, and/or an TCRγ extracellular domain (e.g., TCRγ constant region), the signal peptide is also derived from TCRγ. In some embodiments, when the chimeric receptor polypeptide comprises a transmembrane domain derived from TCRδ, an intracellular domain derived from TCRδ, and/or an TCRδ extracellular domain (e.g., TCRδ constant region), the signal peptide is also derived from TCRδ. In some embodiments, the signal peptide is derived from a molecule different from the TCR subunit which the transmembrane domain and/or intracellular domain of the chimeric receptor polypeptide is derived (or different from the TCR subunit whose extracellular domain is present, adjacent to the transmembrane domain of the chimeric receptor polypeptide, e.g., CD3ε extracellular domain).

In some embodiments, the chimeric receptor polypeptide comprises (or consists essentially of or consists of) from the N-terminus to the C-terminus: optional signal peptide -extracellular target binding domain (e.g., scFv, sdAb, VHH, DARPin, such as anti-BCMA sdAb) - first linker (e.g., GS linker) - extracellular TCR binding domain (e.g., scFv, sdAb, or VHH, such as anti-CD3 scFv or sdAb, or anti-TCR scFv) - second linker (e.g., GS linker) - optional extracellular hinge domain (e.g., CD8 hinge) - transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: option signal peptide - extracellular TCR binding domain (e.g., scFv, sdAb, or VHH, such as anti-CD3 scFv or sdAb, or anti-TCR scFv) - first linker (e.g., GS linker) - extracellular target binding domain (e.g., scFv, sdAb, VHH, DARPin, such as anti-BCMA sdAb) - second linker (e.g., GS linker) - optional extracellular hinge domain (e.g., CD8 hinge) -transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: optional signal peptide -(VHH1 (such as first anti-BCMA sdAb) of extracellular target binding domain - third linker (e.g., GS linker) - VHH2 (such as second anti-BCMA sdAb) of extracellular target binding domain) -first linker (e.g., GS linker) - extracellular TCR binding domain (e.g., scFv, sdAb, or VHH, such as anti-CD3 scFv or sdAb, or anti-TCR scFv) - second linker (e.g., GS linker) - optional extracellular hinge domain (e.g., CD8 hinge) - transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: optional signal peptide - extracellular TCR binding domain (e.g., scFv, sdAb, or VHH, such as anti-CD3 scFv or sdAb, or anti-TCR scFv) - first linker (e.g., GS linker) - (VHH1 (such as first anti-BCMA sdAb) of extracellular target binding domain - third linker (e.g., GS linker) - VHH2 (such as second anti-BCMA sdAb) of extracellular target binding domain) - second linker (e.g., GS linker) - optional extracellular hinge domain (e.g., CD8 hinge) - transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: optional signal peptide - extracellular target binding domain (e.g., scFv, sdAb, VHH, DARPin, such as anti-BCMA sdAb) - first linker (e.g., GS linker) - (VHH1 (such as first anti-CD3 sdAb) of extracellular TCR binding domain - fourth linker (e.g., GS linker) - VHH2 (such as second anti-CD3 sdAb) of extracellular TCR binding domain) - second linker (e.g., GS linker) - optional extracellular hinge domain (e.g., CD8 hinge) - transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: optional signal peptide - (VHH1 (such as first anti-CD3 sdAb) of extracellular TCR binding domain - fourth linker (e.g., GS linker) - VHH2 (such as second anti-CD3 sdAb) of extracellular TCR binding domain) - first linker (e.g., GS linker) - extracellular target binding domain (e.g., scFv, sdAb, VHH, DARPin, such as anti-BCMA sdAb) - second linker (e.g., GS linker) - optional extracellular hinge domain (e.g., CD8 hinge) - transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: optional signal peptide - extracellular target binding domain (e.g., scFv, sdAb, DARPin, such as anti-BCMA sdAb) - first linker (e.g., GS linker) - extracellular TCR binding domain (e.g., scFv, sdAb, or VHH, such as anti-CD3 scFv or sdAb, or anti-TCR scFv) - second linker (e.g., GS linker) - extracellular domain (e.g., of a third TCR subunit, such as non-signal peptide and/or non-variable region of a third TCR subunit) -optional extracellular hinge domain (e.g., CD8 hinge) - transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: option signal peptide - extracellular TCR binding domain (e.g., scFv, sdAb, or VHH, such as anti-CD3 scFv or sdAb, or anti-TCR scFv) - first linker (e.g., GS linker) - extracellular target binding domain (e.g., scFv, sdAb, VHH, DARPin, such as anti-BCMA sdAb) - second linker (e.g., GS linker) - extracellular domain (e.g., of a third TCR subunit, such as non-signal peptide and/or non-variable region of a third TCR subunit) - optional extracellular hinge domain (e.g., CD8 hinge) - transmembrane domain (e.g., of a first TCR subunit) -intracellular domain (e.g., of a second TCR subunit). In some embodiments, the chimeric receptor polypeptide comprises (or consists essentially of or consists of) from the N-terminus to the C-terminus: optional signal peptide - (VHH1 (such as first anti-BCMA sdAb) of extracellular target binding domain - third linker (e.g., GS linker) - VHH2 (such as second anti-BCMA sdAb) of extracellular target binding domain) - first linker (e.g., GS linker) -extracellular TCR binding domain (e.g., scFv, sdAb, or VHH, such as anti-CD3 scFv or sdAb, or anti-TCR scFv) - second linker (e.g., GS linker) - extracellular domain (e.g., of a third TCR subunit, such as non-signal peptide and/or non-variable region of a third TCR subunit) - optional extracellular hinge domain (e.g., CD8 hinge) - transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: optional signal peptide - extracellular TCR binding domain (e.g., scFv, sdAb, or VHH, such as anti-CD3 scFv or sdAb, or anti-TCR scFv) - first linker (e.g., GS linker) - (VHH1 (such as first anti-BCMA sdAb) of extracellular target binding domain - third linker (e.g., GS linker) - VHH2 (such as second anti-BCMA sdAb) of extracellular target binding domain) - second linker (e.g., GS linker) - extracellular domain (e.g., of a third TCR subunit, such as non-signal peptide and/or non-variable region of a third TCR subunit) - optional extracellular hinge domain (e.g., CD8 hinge) - transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: optional signal peptide - extracellular target binding domain (e.g., scFv, sdAb, VHH, DARPin, such as anti-BCMA sdAb) - first linker (e.g., GS linker) - (VHH1 (such as first anti-CD3 sdAb) of extracellular TCR binding domain - fourth linker (e.g., GS linker) - VHH2 (such as second anti-CD3 sdAb) of extracellular TCR binding domain) - second linker (e.g., GS linker) - extracellular domain (e.g., of a third TCR subunit, such as non-signal peptide and/or non-variable region of a third TCR subunit) - optional extracellular hinge domain (e.g., CD8 hinge) - transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: optional signal peptide - (VHH1 (such as first anti-CD3 sdAb) of extracellular TCR binding domain - fourth linker (e.g., GS linker) - VHH2 (such as second anti-CD3 sdAb) of extracellular TCR binding domain) - first linker (e.g., GS linker) - extracellular target binding domain (e.g., scFv, sdAb, VHH, DARPin, such as anti-BCMA sdAb) - second linker (e.g., GS linker) -extracellular domain (e.g., of a third TCR subunit, such as non-signal peptide and/or non-variable region of a third TCR subunit) - optional extracellular hinge domain (e.g., CD8 hinge) -transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: optional signal peptide -(VHH1 (such as first anti-BCMA sdAb) of extracellular target binding domain - third linker (e.g., GS linker) - VHH2 (such as second anti-BCMA sdAb) of extracellular target binding domain) -first linker (e.g., GS linker) - (VHH1 (such as first anti-CD3 sdAb) of extracellular TCR binding domain - fourth linker (e.g., GS linker) - VHH2 (such as second anti-CD3 sdAb) of extracellular TCR binding domain) - second linker (e.g., GS linker) - optional extracellular hinge domain (e.g., CD8 hinge) - transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: optional signal peptide - (VHH1 (such as first anti-CD3 sdAb) of extracellular TCR binding domain - fourth linker (e.g., GS linker) - VHH2 (such as second anti-CD3 sdAb) of extracellular TCR binding domain) - first linker (e.g., GS linker) - (VHH1 (such as first anti-BCMA sdAb) of extracellular target binding domain - third linker (e.g., GS linker) - VHH2 (such as second anti-BCMA sdAb) of extracellular target binding domain) - second linker (e.g., GS linker) - optional extracellular hinge domain (e.g., CD8 hinge) - transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: optional signal peptide - (VHH1 (such as first anti-BCMA sdAb) of extracellular target binding domain - third linker (e.g., GS linker) - VHH2 (such as second anti-BCMA sdAb) of extracellular target binding domain) - first linker (e.g., GS linker) - (VHH1 (such as first anti-CD3 sdAb) of extracellular TCR binding domain - fourth linker (e.g., GS linker) - VHH2 (such as second anti-CD3 sdAb) of extracellular TCR binding domain) - second linker (e.g., GS linker) - extracellular domain (e.g., of a third TCR subunit, such as non-signal peptide and/or non-variable region of a third TCR subunit) - optional extracellular hinge domain (e.g., CD8 hinge) - transmembrane domain (e.g., of a first TCR subunit) - intracellular domain (e.g., of a second TCR subunit). In some embodiments, the chimeric receptor polypeptide comprises (or consists essentially of or consists of) from the N-terminus to the C-terminus: optional signal peptide - (VHH1 (such as first anti-CD3 sdAb) of extracellular TCR binding domain - fourth linker (e.g., GS linker) - VHH2 (such as second anti-CD3 sdAb) of extracellular TCR binding domain) - first linker (e.g., GS linker) - (VHH1 (such as first anti-BCMA sdAb) of extracellular target binding domain - third linker (e.g., GS linker) - VHH2 (such as second anti-BCMA sdAb) of extracellular target binding domain) - second linker (e.g., GS linker) -extracellular domain (e.g., of a third TCR subunit, such as non-signal peptide and/or non-variable region of a third TCR subunit) - optional extracellular hinge domain (e.g., CD8 hinge) -transmembrane domain (e.g., of a first TCR subunit) - intracellular domain (e.g., of a second TCR subunit). In some embodiments, the first, second, and third TCR subunits are the same. In some embodiments, the first, second, and/or third TCR subunits are different. The first, second, third, and fourth linker can be any of the linkers described herein. In some embodiments, the first, second, third, and/or fourth linker is a GS linker, an α-helical linker, a glycine-alanine polymer linker, an alanine-serine polymer linker, or an IgG4-Fc linker. In some embodiments, the first, second, third, and/or fourth linker comprises the amino acid sequence of any of SEQ ID NOs: 1-21, 67, and 68.

### Nucleic Acids Encoding the Chimeric Receptor Polynucleotides

Nucleic acid molecules (such as isolated nucleic acids) encoding the chimeric receptor polypeptides described herein are also contemplated, hereinafter also referred to as “STS polypeptide-encoding nucleic acid” or “STS-encoding nucleic acid”. The present invention also provides vectors in which a nucleic acid of the present invention is inserted.

For example, in some embodiments, there is provided a nucleic acid (e.g., isolated nucleic acid) encoding a chimeric receptor polypeptide comprising (or consisting essentially of or consisting of): a) an extracellular target binding domain comprising a target antigen binding domain (such as scFv, sdAb, or DARPin) specifically binding to a target antigen (e.g., BCMA); b) an extracellular TCR binding domain comprising a TCR antigen binding domain (such as scFv or sdAb) specifically binding to a TCR subunit (e.g., extracellular domain of a TCR subunit, such as extracellular domain of CD3ε or TCR); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided a nucleic acid (e.g., isolated nucleic acid) encoding a chimeric receptor polypeptide comprising (or consisting essentially of or consisting of), from N′ to C′: a) an optional signal peptide; b) an extracellular target binding domain comprising a target antigen binding domain (such as scFv, sdAb, or DARPin) specifically binding to a target antigen (e.g., BCMA); c) an optional first linker (e.g., GS linker); d) an extracellular TCR binding domain comprising a TCR antigen binding domain (such as scFv or sdAb) specifically binding to a TCR subunit (e.g., extracellular domain of a TCR subunit, such as extracellular domain of CD3ε or TCR); e) an optional second linker (e.g., GS linker); f) an optional extracellular domain of a third TCR subunit (e.g., non-signal peptide and/or non-variable region of a third TCR subunit); g) an optional hinge region (e.g., CD8 hinge); h) a transmembrane domain comprising a transmembrane domain of a first TCR subunit; and i) an intracellular domain comprising an intracellular domain of a second TCR subunit, wherein the first, second, and third TCR subunits are all selected from the group consisting of CD3ε, CD3γ, CD3δ, TCRα, TCRβ, TCRγ, and TCRδ. In some embodiments, there is provided a nucleic acid (e.g., isolated nucleic acid) encoding a chimeric receptor polypeptide comprising (or consisting essentially of or consisting of), from N′ to C′: a) an optional signal peptide; b) an extracellular TCR binding domain comprising a TCR antigen binding domain (such as scFv or sdAb) specifically binding to a TCR subunit (e.g., extracellular domain of a TCR subunit, such as extracellular domain of CD3ε or TCR); c) an optional first linker (e.g., GS linker); d) an extracellular target binding domain comprising a target antigen binding domain (such as scFv, sdAb, or DARPin) specifically binding to a target antigen (e.g., BCMA); e) an optional second linker (e.g., GS linker); f) an optional extracellular domain of a third TCR subunit (e.g., non-signal peptide and/or non-variable region of a third TCR subunit); g) an optional hinge region (e.g., CD8 hinge); h) a transmembrane domain comprising a transmembrane domain of a first TCR subunit; and i) an intracellular domain comprising an intracellular domain of a second TCR subunit, wherein the first, second, and third TCR subunits are all selected from the group consisting of CD3ε, CD3γ, CD3δ, TCRα, TCRβ, TCRγ, and TCRδ. In some embodiments, the extracellular TCR binding domain comprises two or more TCR antigen binding domains (e.g., scFv or sdAb) arranged in tandem. In some embodiments, the extracellular target binding domain comprises two or more target antigen binding domains (e.g., scFv, sdAb, or DARPin) arranged in tandem. In some embodiments, the first, second, and third TCR subunits are all the same. In some embodiments, the first, second, and/or third TCR subunits are different. In some embodiments, there is provided a nucleic acid (e.g., isolated nucleic acid) encoding a chimeric receptor polypeptide comprising (or consisting essentially of or consisting of) the amino acid sequence of any of SEQ ID NOs: 43-48, 51-58, and 61-63.

In some embodiments, there is provided a vector (such as viral vector, e.g., lentiviral vector) comprising any of the nucleic acids described herein. In some embodiments, the vector comprises two or more (such as two) any of the nucleic acids described herein. In some embodiments, the two nucleic acids each has a separate promoter (can be the same or different). In some embodiments, the two nucleic acids are regulated under the same promoter (e.g., hEF1α). In some embodiments, the two or more (such as two) STS-encoding nucleic acids are connected via one or more linking sequences, such as any of nucleic acids encoding P2A, T2A, E2A, F2A, BmCPV 2A, and BmIFV 2A, and internal ribosome entry site (IRES) sequence. Thus in some embodiments, there is provided a vector (such as viral vector, e.g., lentiviral vector) comprising a) a promoter; b) a first nucleic acid encoding a first chimeric receptor polypeptide; c) a linking sequence (e.g., IRES, or nucleic acid encoding any of P2A, T2A, E2A, F2A, BmCPV 2A, and BmIFV 2A); and d) a second nucleic acid encoding a second chimeric receptor polypeptide; wherein the first chimeric receptor polypeptide comprises (or consists essentially of or consists of): a) a first extracellular target binding domain comprising a first target antigen binding domain (such as scFv, sdAb, or DARPin) specifically binding to a first target antigen (e.g., BCMA); b) a first extracellular TCR binding domain comprising a first TCR antigen binding domain (such as scFv or sdAb) specifically binding to a fifth TCR subunit (e.g., extracellular domain of a fifth TCR subunit, such as extracellular domain of CD3ε or TCR); c) a first transmembrane domain comprising a transmembrane domain of a first TCR subunit; and d) a first intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ; and wherein the second chimeric receptor polypeptide comprises (or consists essentially of or consists of): a) a second extracellular target binding domain comprising a second target antigen binding domain (such as scFv, sdAb, or DARPin) specifically binding to a second target antigen (e.g., BCMA); b) a second extracellular TCR binding domain comprising a second TCR antigen binding domain (such as scFv or sdAb) specifically binding to a sixth TCR subunit (e.g., extracellular domain of a sixth TCR subunit, such as extracellular domain of CD3ε or TCR); c) a second transmembrane domain comprising a transmembrane domain of a third TCR subunit; and d) a second intracellular domain comprising an intracellular domain of a fourth TCR subunit, wherein the third TCR subunit and the fourth TCR subunit are both selected from the group consisting of CD3ε, CD3γ, CD3δ, TCRα, TCRβ, TCRγ, and TCRδ. In some embodiments, the first chimeric receptor polypeptide and the second chimeric receptor polypeptide have the same domain arrangement (e.g., extracellular target binding domains are both at the N-terminus of extracellular TCR binding domains, extracellular target binding domains are both at the C-terminus of extracellular TCR binding domains, both comprise or both do not comprise extracellular domain of a TCR subunit, and/or both comprise or both do not comprise hinge region). In some embodiments, the first chimeric receptor polypeptide and the second chimeric receptor polypeptide have different domain arrangement (e.g., one comprises extracellular target binding domains at the N-terminus of the extracellular TCR binding domain and the other one comprises extracellular target binding domains at the C-terminus of the extracellular TCR binding domain, two chimeric receptor polypeptides have different numbers of target antigen binding domains and/or TCR antigen binding domains, one comprises extracellular domain of a TCR subunit while the other one does not, and/or one comprises hinge region while the other one does not). Thus in some embodiments, there is provided a vector (such as viral vector, e.g., lentiviral vector) comprising a) a promoter; b) a first nucleic acid encoding a first chimeric receptor polypeptide; c) a linking sequence (e.g., IRES, or nucleic acid encoding any of P2A, T2A, E2A, F2A, BmCPV 2A, and BmIFV 2A); and d) a second nucleic acid encoding a second chimeric receptor polypeptide; wherein the first chimeric receptor polypeptide comprises (or consists essentially of or consists of) from N′ to C′: a) an optional first signal peptide; b) a first extracellular target binding domain comprising a first target antigen binding domain (such as scFv, sdAb, or DARPin) specifically binding to a first target antigen (e.g., BCMA); c) an optional first linker (e.g., GS linker); d) a first extracellular TCR binding domain comprising a first TCR antigen binding domain (such as scFv or sdAb) specifically binding to a fifth TCR subunit (e.g., extracellular domain of a fifth TCR subunit, such as extracellular domain of CD3ε or TCR); e) an optional second linker (e.g., GS linker); f) an optional extracellular domain of a seventh TCR subunit (e.g., non-signal peptide and/or non-variable region of a seventh TCR subunit); g) an optional first hinge region (e.g., CD8 hinge); h) a first transmembrane domain comprising a transmembrane domain of a first TCR subunit; and i) a first intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ; and wherein the second chimeric receptor polypeptide comprises (or consists essentially of or consists of) from N′ to C′: a) an optional second signal peptide; b) a second extracellular target binding domain comprising a second target antigen binding domain (such as scFv, sdAb, or DARPin) specifically binding to a second target antigen (e.g., BCMA); c) an optional third linker (e.g., GS linker); d) a second extracellular TCR binding domain comprising a second TCR antigen binding domain (such as scFv or sdAb) specifically binding to a sixth TCR subunit (e.g., extracellular domain of a sixth TCR subunit, such as extracellular domain of CD3ε or TCR); e) an optional fourth linker (e.g., GS linker); f) an optional extracellular domain of a eighth TCR subunit (e.g., non-signal peptide and/or non-variable region of an eighth TCR subunit); g) an optional second hinge region (e.g., CD8 hinge); h) a second transmembrane domain comprising a transmembrane domain of a third TCR subunit; and i) a second intracellular domain comprising an intracellular domain of a fourth TCR subunit, wherein the third TCR subunit and the fourth TCR subunit are both selected from the group consisting of CD3ε, CD3γ, CD3δ, TCRα, TCRβ, TCRγ, and TCRδ. In some embodiments, there is provided a vector (such as viral vector, e.g., lentiviral vector) comprising a) a promoter; b) a first nucleic acid encoding a first chimeric receptor polypeptide; c) a linking sequence (e.g., IRES, or nucleic acid encoding any of P2A, T2A, E2A, F2A, BmCPV 2A, and BmIFV 2A); and d) a second nucleic acid encoding a second chimeric receptor polypeptide; wherein the first chimeric receptor polypeptide comprises (or consists essentially of or consists of) from N′ to C′: a) an optional first signal peptide; b) a first extracellular TCR binding domain comprising a first TCR antigen binding domain (such as scFv or sdAb) specifically binding to a fifth TCR subunit (e.g., extracellular domain of a fifth TCR subunit, such as extracellular domain of CD3ε or TCR); c) an optional first linker (e.g., GS linker); d) a first extracellular target binding domain comprising a first target antigen binding domain (such as scFv, sdAb, or DARPin) specifically binding to a first target antigen (e.g., BCMA); e) an optional second linker (e.g., GS linker); f) an optional extracellular domain of a seventh TCR subunit (e.g., non-signal peptide and/or non-variable region of a seventh TCR subunit); g) an optional first hinge region (e.g., CD8 hinge); h) a first transmembrane domain comprising a transmembrane domain of a first TCR subunit; and i) a first intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ; and wherein the second chimeric receptor polypeptide comprises (or consists essentially of or consists of) from N′ to C′: a) an optional second signal peptide; b) a second extracellular TCR binding domain comprising a second TCR antigen binding domain (such as scFv or sdAb) specifically binding to a sixth TCR subunit (e.g., extracellular domain of a sixth TCR subunit, such as extracellular domain of CD3ε or TCR); c) an optional third linker (e.g., GS linker); d) a second extracellular target binding domain comprising a second target antigen binding domain (such as scFv, sdAb, or DARPin) specifically binding to a second target antigen (e.g., BCMA); e) an optional fourth linker (e.g., GS linker); f) an optional extracellular domain of a eighth TCR subunit (e.g., non-signal peptide and/or non-variable region of an eighth TCR subunit); g) an optional second hinge region (e.g., CD8 hinge); h) a second transmembrane domain comprising a transmembrane domain of a third TCR subunit; and i) a second intracellular domain comprising an intracellular domain of a fourth TCR subunit, wherein the third TCR subunit and the fourth TCR subunit are both selected from the group consisting of CD3ε, CD3γ, CD3δ, TCRα, TCRβ, TCRγ, and TCRδ. In some embodiments, the first, second, third, fourth, fifth, sixth, seventh, and eighth TCR subunits are the same. In some embodiments, the first, second, third, fourth, fifth, sixth, seventh, and/or eighth TCR subunits are different. In some embodiments, the third, fourth, and eighth TCR subunits are the same. In some embodiments, the first, second, and seventh TCR subunits are the same. In some embodiments, the third, fourth, and eighth TCR subunits are the same (e.g., TCRα), and the third, fourth, and eighth TCR subunits are the same (e.g., TCRβ), but the third, fourth, and eighth TCR subunits are different from the third, fourth, and eighth TCR subunits. In some embodiments, the third, fourth, and eighth TCR subunits are TCRα, and the third, fourth, and eighth TCR subunits are TCRβ. In some embodiments, the third, fourth, and eighth TCR subunits are TCRγ, and the third, fourth, and eighth TCR subunits are TCRδ. In some embodiments, the first, second, third, and fourth linkers are the same. In some embodiments, the first, second, third, and/or fourth linkers are different. In some embodiments, the first, second, third, and fourth linkers are selected from a GS linker, an α-helical linker, a glycine-alanine polymer linker, an alanine-serine polymer linker, and an IgG4-Fc linker. In some embodiments, the first, second, third, and fourth linkers are selected from the group consisting of SEQ ID NOs: 1-21, 67, and 68. In some embodiments, the first and second TCR antigen binding domains are the same. In some embodiments, the first and second TCR antigen binding domains are different. In some embodiments, the first and/or second TCR antigen binding domain is an anti-CD3 sdAb comprising the amino acid sequence of SEQ ID NO: 22 or 23. In some embodiments, the first and/or second TCR antigen binding domain is an anti-CD3 scFv comprising the amino acid sequence of SEQ ID NO: 24. In some embodiments, the first and/or second TCR antigen binding domain is an anti-TCR scFv comprising the amino acid sequence of SEQ ID NO: 27. In some embodiments, the first and second target antigen binding domains are the same. In some embodiments, the first and second target antigen binding domains are different. In some embodiments, the first and/or second target antigen binding domain is an sdAb specifically binding to BCMA, such as any of the anti-BCMA sdAbs disclosed in PCT/CN2016/094408 and PCT/CN2017/096938. In some embodiments, the first and/or second target antigen binding domain is an anti-BCMA sdAb comprising the amino acid sequence of SEQ ID NO: 26.

In some embodiments, there is provided a vector (such as viral vector, e.g., lentiviral vector) comprising a nucleic acid encoding a polypeptide, wherein the polypeptide comprises (or consists essentially of or consists of) from the N-terminus to the C-terminus: optional first signal peptide - first anti-BCMA sdAb (e.g., SEQ ID NO: 26) - first linker (e.g, GS linker) - first anti-CD3 scFv (e.g., SEQ ID NO: 24) - second linker (e.g, GS linker) - TCRα constant region -TCRα transmembrane domain - TCRα intracellular domain - P2A - optional second signal peptide - second anti-BCMA sdAb (e.g., SEQ ID NO: 26) - third linker (e.g, GS linker) -second anti-CD3 scFv (e.g., SEQ ID NO: 24) - fourth linker (e.g, GS linker) - TCRβ constant region - TCRβ transmembrane domain - TCRβ intracellular domain. In some embodiments, there is provided a vector (such as viral vector, e.g., lentiviral vector) comprising a nucleic acid encoding a polypeptide, wherein the polypeptide comprises (or consists essentially of or consists of) from the N-terminus to the C-terminus: optional first signal peptide - first anti-BCMA sdAb (e.g., SEQ ID NO: 26) - first linker (e.g, GS linker) - first anti-CD3 scFv (e.g., SEQ ID NO: 24) - second linker (e.g, GS linker) - TCRα transmembrane domain - TCRα intracellular domain -P2A - optional second signal peptide - second anti-BCMA sdAb (e.g., SEQ ID NO: 26) - third linker (e.g, GS linker) - second anti-CD3 scFv (e.g., SEQ ID NO: 24) - fourth linker (e.g, GS linker) - TCRβ transmembrane domain - TCRβ intracellular domain. In some embodiments, there is provided a vector (such as viral vector, e.g., lentiviral vector) comprising a nucleic acid encoding a polypeptide, wherein the polypeptide comprises (or consists essentially of or consists of) from the N-terminus to the C-terminus: optional first signal peptide - first anti-BCMA sdAb (e.g., SEQ ID NO: 26) - first linker (e.g, GS linker) - first anti-CD3 scFv (e.g., SEQ ID NO: 24) - second linker (e.g, GS linker) - TCRγ constant region - TCRγ transmembrane domain - TCRγ intracellular domain - P2A - optional second signal peptide - second anti-BCMA sdAb (e.g., SEQ ID NO: 26) - third linker (e.g, GS linker) - second anti-CD3 scFv (e.g., SEQ ID NO: 24) -fourth linker (e.g, GS linker) - TCRδ constant region - TCRδ transmembrane domain - TCRδ intracellular domain. In some embodiments, there is provided a vector (such as viral vector, e.g., lentiviral vector) comprising a nucleic acid encoding a polypeptide, wherein the polypeptide comprises (or consists essentially of or consists of) from the N-terminus to the C-terminus: optional first signal peptide - first anti-BCMA sdAb (e.g., SEQ ID NO: 26) - first linker (e.g, GS linker) - first anti-CD3 scFv (e.g., SEQ ID NO: 24) - second linker (e.g, GS linker) - TCRγ transmembrane domain - TCRγ intracellular domain - P2A - optional second signal peptide -second anti-BCMA sdAb (e.g., SEQ ID NO: 26) - third linker (e.g, GS linker) - second anti-CD3 scFv (e.g., SEQ ID NO: 24) - fourth linker (e.g, GS linker) - TCRδ transmembrane domain - TCRδ intracellular domain. In some embodiments, there is provided a vector (such as viral vector, e.g., lentiviral vector) comprising a nucleic acid encoding a polypeptide, wherein the polypeptide comprises (or consists essentially of or consists of) from the N-terminus to the C-terminus: optional first signal peptide - first anti-BCMA sdAb (e.g., SEQ ID NO: 26) - first linker (e.g, GS linker) - first anti-TCR scFv (e.g., SEQ ID NO: 27) - second linker (e.g, GS linker) - TCRα constant region - TCRα transmembrane domain - TCRα intracellular domain -P2A - optional second signal peptide - second anti-BCMA sdAb (e.g., SEQ ID NO: 26) - third linker (e.g, GS linker) - second anti-TCR scFv (e.g., SEQ ID NO: 27) - fourth linker (e.g, GS linker) - TCRβ constant region - TCRβ transmembrane domain - TCRβ intracellular domain. In some embodiments, there is provided a vector (such as viral vector, e.g., lentiviral vector) comprising a nucleic acid encoding a polypeptide, wherein the polypeptide comprises (or consists essentially of or consists of) from the N-terminus to the C-terminus: optional first signal peptide - first anti-BCMA sdAb (e.g., SEQ ID NO: 26) - first linker (e.g, GS linker) - first anti-TCR scFv (e.g., SEQ ID NO: 27) - second linker (e.g, GS linker) - TCRα transmembrane domain - TCRα intracellular domain - P2A - optional second signal peptide - second anti-BCMA sdAb (e.g., SEQ ID NO: 26) - third linker (e.g, GS linker) - second anti-TCR scFv (e.g., SEQ ID NO: 27) - fourth linker (e.g, GS linker) - TCRβ transmembrane domain - TCRβ intracellular domain. In some embodiments, there is provided a vector (such as viral vector, e.g., lentiviral vector) comprising a nucleic acid encoding a polypeptide, wherein the polypeptide comprises (or consists essentially of or consists of) from the N-terminus to the C-terminus: optional first signal peptide - first anti-BCMA sdAb (e.g., SEQ ID NO: 26) - first linker (e.g, GS linker) - first anti-TCR scFv (e.g., SEQ ID NO: 27) - second linker (e.g, GS linker) - TCRγ constant region - TCRγ transmembrane domain - TCRγ intracellular domain - P2A - optional second signal peptide - second anti-BCMA sdAb (e.g., SEQ ID NO: 26) - third linker (e.g, GS linker) - second anti-TCR scFv (e.g., SEQ ID NO: 27) - fourth linker (e.g, GS linker) - TCRδ constant region - TCRδ transmembrane domain - TCRδ intracellular domain. In some embodiments, there is provided a vector (such as viral vector, e.g., lentiviral vector) comprising a nucleic acid encoding a polypeptide, wherein the polypeptide comprises (or consists essentially of or consists of) from the N-terminus to the C-terminus: optional first signal peptide - first anti-BCMA sdAb (e.g., SEQ ID NO: 26) - first linker (e.g, GS linker) - first anti-TCR scFv (e.g., SEQ ID NO: 27) - second linker (e.g, GS linker) - TCRγ transmembrane domain - TCRγ intracellular domain - P2A - optional second signal peptide - second anti-BCMA sdAb (e.g., SEQ ID NO: 26) - third linker (e.g, GS linker) - second anti-TCR scFv (e.g., SEQ ID NO: 27) -fourth linker (e.g, GS linker) - TCRδ transmembrane domain - TCRδ intracellular domain. In some embodiments, the first, second, third, and/or fourth linkers are different. In some embodiments, the first, second, third, and fourth linkers are selected from a GS linker, an α-helical linker, a glycine-alanine polymer linker, an alanine-serine polymer linker, and an IgG4-Fc linker. In some embodiments, the first, second, third, and fourth linkers are selected from the group consisting of SEQ ID NOs: 1-21, 67, and 68. In some embodiments, the first and second anti-CD3 scFvs are the same. In some embodiments, the first and second anti-CD3 scFvs are different. In some embodiments, the first and second anti-TCR scFvs are the same. In some embodiments, the first and second anti-TCR scFvs are different. In some embodiments, the first and/or second anti-CD3 scFv comprises the amino acid sequence of SEQ ID NO: 24. In some embodiments, the first and/or second anti-TCR scFv comprises the amino acid sequence of SEQ ID NO: 27. In some embodiments, the first and second anti-BCMA sdAbs are the same. In some embodiments, the first and second anti-BCMA sdAbs are different. In some embodiments, the first and/or second anti-BCMA sdAb is any of the anti-BCMA sdAbs disclosed in PCT/CN2016/094408 and PCT/CN2017/096938. In some embodiments, the first and/or second target anti-BCMA sdAb comprises the amino acid sequence of SEQ ID NO: 26.

In brief summary, the expression of an chimeric receptor polypeptide by a nucleic acid encoding the chimeric receptor polypeptide can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to, a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In some embodiments, the lentiviral vector is an H1V-1-based lentiviral vector, such as pLVX-Puro or modified lentiviral vector thereof.

Additional promoter-type elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The efficiencies of such constitutive promoters on driving transgene expression have been widely compared in a huge number of studies. For example, Michael C. Milone et al compared the efficiencies of CMV, hEF1α, UbiC and PGK to drive chimeric receptor expression in primary human T cells, and concluded that hEF1α promoter not only induced the highest level of transgene expression, but was also optimally maintained in the CD4 and CD8 human T cells (Molecular Therapy, 17(8): 1453-1464 (2009)).

Inducible promoters are also contemplated as part of the invention. The use of an inducible promoter provides a molecular switch capable of turning on expression of the polynucleotide sequence which it is operatively linked when such expression is desired, or turning off the expression when expression is not desired. Exemplary inducible promoter systems for use in eukaryotic cells include, but are not limited to, hormone-regulated elements, synthetic ligand-regulated elements and ionizing radiation-regulated elements. Further exemplary inducible promoter systems for use in in vitro or in vivo mammalian systems are reviewed in Gingrich et al. (1998) Annual Rev. Neurosci 21:377-405.

An exemplary inducible promoter system for use in the present invention is the Tet system. In an exemplary embodiment, a polynucleotide of interest is under the control of a promoter that comprises one or more Tet operator (TetO) sites. In the inactive state, Tet repressor (TetR) will bind to the TetO sites and repress transcription from the promoter. In the active state, e.g., in the presence of an inducing agent such as tetracycline (Tc), anhydrotetracycline, doxycycline (Dox), or an active analog thereof, the inducing agent causes release of TetR from TetO, thereby allowing transcription to take place. Doxycycline is a member of the tetracycline family of antibiotics having the chemical name of 1-dimethylamino-2,4a,5,7,12-pentahydroxy-11-methyl-4,6-dioxo-1,4a,11,11a,12,12a-hexahydrotetracene-3-carboxamide.

In one embodiment, a TetR is codon-optimized for expression in mammalian cells, e.g., murine or human cells. Most amino acids are encoded by more than one codon due to the degeneracy of the genetic code, allowing for substantial variations in the nucleotide sequence of a given nucleic acid without any alteration in the amino acid sequence encoded by the nucleic acid. However, many organisms display differences in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism (e.g., a prokaryote) may be tailored for improved expression in a different organism (e.g., a eukaryote) through codon optimization.

Other specific variations of the Tet system include the following “Tet-Off” and “Tet-On” systems. In the Tet-Off system, transcription is inactive in the presence of Tc or Dox. In that system, a tetracycline-controlled transactivator protein (tTA), which is composed of TetR fused to the strong transactivating domain of VP16 from Herpes simplex virus, regulates expression of a target nucleic acid that is under transcriptional control of a tetracycline-responsive promoter element (TRE). The TRE is made up of TetO sequence concatamers fused to a promoter (commonly the minimal promoter sequence derived from the human cytomegalovirus (hCMV) immediate-early promoter). In the absence of Tc or Dox, tTA binds to the TRE and activates transcription of the target gene. In the presence of Tc or Dox, tTA cannot bind to the TRE, and expression from the target gene remains inactive.

Conversely, in the Tet-On system, transcription is active in the presence of Tc or Dox. The Tet-On system is based on a reverse tetracycline-controlled transactivator, rtTA. Like tTA, rtTA is a fusion protein comprised of the TetR repressor and the VP16 transactivation domain. However, a four amino acid change in the TetR DNA binding moiety alters rtTA’s binding characteristics such that it can only recognize the tetO sequences in the TRE of the target transgene in the presence of Dox. Thus, in the Tet-On system, transcription of the TRE-regulated target gene is stimulated by rtTA only in the presence of Dox.

Another inducible promoter system is the lac repressor system from E. coli. (See, Brown et al., Cell 49:603-612 (1987). The lac repressor system functions by regulating transcription of a polynucleotide of interest operably linked to a promoter comprising the lac operator (lacO). The lac repressor (lacR) binds to LacO, thus preventing transcription of the polynucleotide of interest. Expression of the polynucleotide of interest is induced by a suitable inducing agent, e.g., isopropyl-β-D-thiogalactopyranoside (IPTG).

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, there is provided a nucleic acid (e.g., isolated nucleic acid) encoding a chimeric receptor polypeptide according to any of the chimeric receptor polypeptides described herein. Expression can be determined at the mRNA or protein level. The level of mRNA expression can be determined by measuring the amount of mRNA transcribed from the nucleic acid using various well-known methods, including Northern blotting, quantitative RT-PCR, microarray analysis and the like. The level of protein expression can be measured by known methods including immunocytochemical staining, enzyme-linked immunosorbent assay (ELISA), western blot analysis, luminescent assays, mass spectrometry, high performance liquid chromatography, high-pressure liquid chromatography-tandem mass spectrometry, and the like.

In some embodiments, there is provided a vector (such as a lentiviral vector) comprising nucleic acid encoding an chimeric receptor polypeptide according to any of the chimeric receptor polypeptides described herein, comprising a promoter operably linked to a nucleic acid sequence encoding the chimeric receptor polypeptide. In some embodiments, the promoter is inducible. In some embodiments, the promoter is constitutive. In some embodiments, the promoter is hEF1α or cytomegalovirus immediate early promoter (P_{CMV IE}).

In some embodiments, there is provided a vector (such as a lentiviral vector) comprising a first nucleic acid encoding a chimeric receptor polypeptide according to any of the chimeric receptor polypeptides described herein, and a second nucleic acid encoding a chimeric antigen receptor (CAR) or an engineered TCR. In some embodiments, there is provided a vector (such as a lentiviral vector) comprising a first nucleic acid encoding a first chimeric receptor polypeptide according to any of the chimeric receptor polypeptides described herein, and a second nucleic acid encoding a second chimeric receptor polypeptide according to any of the chimeric receptor polypeptides described herein. In some embodiments, the two nucleic acids each has a separate promoter. In some embodiments, the two nucleic acids are under control of the same promoter (such as inducible promoter), and the two nucleic acids are connected via a linking sequence. In some embodiments, the linking sequence encodes a self-cleaving 2A peptide, such as P2A, T2A, E2A, F2A, BmCPV 2A, BmIFV 2A. In some embodiments, the linking sequence encodes a P2A peptide (e.g., comprises SEQ ID NO: 66) or a T2A peptide. In some embodiments, the linking sequence is an internal ribosome entry site (IRES). IRES is an RNA element that allows for translation initiation in a cap-independent manner. In some embodiments, the promoter is inducible. In some embodiments, the promoter is constitutive (e.g., hEF1α). In some embodiments, the first nucleic acid encoding a chimeric receptor polypeptide according to any of the chimeric receptor polypeptides described herein, and the second nucleic acid encoding a chimeric antigen receptor (CAR) or an engineered TCR are on separate vectors. In some embodiments, the first nucleic acid encoding a first chimeric receptor polypeptide according to any of the chimeric receptor polypeptides described herein, and the second nucleic acid encoding a second chimeric receptor polypeptide according to any of the chimeric receptor polypeptides described herein are on separate vectors.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human, cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In some embodiments, the vector is a non-viral vector. In some embodiments, the vector is a transposon, such as a Sleeping Beauty (SB) transposon system, or a PiggyBac transposon system. In some embodiments, the vector is a polymer-based non-viral vector, including for example, poly(lactic-co-glycolic acid) (PLGA) and poly lactic acid (PLA), poly(ethylene imine) (PEI), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as poly-L-lysine. Any of the known non-viral vectors suitable for genome editing can be used for introducing the nucleic acid described herein to the immune cell (e.g., T cell). See, for example, Yin H. et al. Nature Rev. Genetics (2014) 15:521-555; Aronovich EL et al. “The Sleeping Beauty transposon system: a non-viral vector for gene therapy.” Hum. Mol. Genet. (2011) R1: R14-20; and Zhao S. et al. “PiggyBac transposon vectors: the tools of the human gene editing.” Transl. Lung Cancer Res. (2016) 5(1): 120-125, which are incorporated herein by reference. In some embodiments, the nucleic acid encoding any of chimeric receptor polypeptides described herein are introduced to the immune cell (e.g., T cell) by a physical method, including, but not limited to electroporation, sonoporation, photoporation, magnetofection, hydroporation.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In some embodiments, the Sleeping Beauty Transposons system is used as a non-viral vectors. The Sleeping Beauty Transposon system was engineered such that DNA transposons precisely insert defined DNA sequences almost randomly into host genomes thereby increasing the longevity of gene expression (even through multiple generations). Moreover, transposition avoids the formation of multiple, tandem integrations, which often results in switching off expression of the transgene. Aronovich et al. (April 2011). “The Sleeping Beauty transposon system: a non-viral vector for gene therapy”. Hum Mol Genet. 20 (R1): R14-R20.

In some embodiments, the PiggyBac (PB) transposon system is used as a non-viral vector. The PB transposon system transposes between vectors and chromosomes via a “cut and paste” mechanism. During transposition, the PB transposase recognizes transposon-specific inverted terminal repeats (ITRs) sequences located on both ends of the transposon vector and moves the contents from its original positions for integration into TTAA chromosomal sites.

A variety of assays may be performed to confirm the presence of the recombinant DNA sequence in the host cell. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Immune Cells Expressing the Chimeric Receptor Polypeptides (STS Polypeptides)

In some embodiments, there is provided an immune cell, or engineered mammalian immune cells (such as a T cell) expressing on its surface a chimeric receptor polypeptide according to any of the chimeric receptor polypeptides described herein, hereinafter also referred to as “STS-immune cells” or “STS-T cells.” In some embodiments, the immune cell (such as a T cell) comprises two or more chimeric receptor polypeptides. In some embodiments, the immune cell (e.g., T cell) comprises a nucleic acid (or vector thereof) encoding any of the chimeric receptor polypeptides described herein, wherein the chimeric receptor polypeptide is expressed from the nucleic acid and localized to the immune cell surface. In some embodiments, the immune cell (such as a T cell) further expresses a CAR or an engineered TCR on its cell surface. In some embodiments, the immune cell (such as a T cell) further comprises a second nucleic acid (on the same vector as the nucleic acid encoding the chimeric receptor polypeptide, or on a different vector) encoding a CAR or an engineered TCR. In some embodiments, the chimeric receptor polypeptide described herein does not affect the expression of endogenous TCR complex (e.g., endogenous TCRα/β and/or CD3 expression) of the engineered immune cell (e.g., T cell).

Exemplary immune cells useful for the present invention include, but are not limited to, dendritic cells (including immature dendritic cells and mature dendritic cells), T lymphocytes (such as naive T cells, effector T cells, memory T cells, cytotoxic T lymphocytes, T helper cells, Natural Killer T cells, Treg cells, tumor infiltrating lymphocytes (TIL)), monocytes, granulocytes, and combinations thereof. Subpopulations of immune cells can be defined by the presence or absence of one or more cell surface markers known in the art (e.g., CD3, CD4, CD8, CD19, CD20, CD11c, CD123, CD56, CD34, CD14, CD33, etc.). In some embodiments, the immune cell is selected from the group consisting of: Tαβ cells, Tγδ cells, effector T cells, memory T cells, cytotoxic T cells, T helper cells, Natural Killer T cells, Treg cells, tumor infiltrating lymphocytes (TIL). In some embodiments, the immune cell is an effector T cell.

“Immune effector cells” are immune cells that can perform immune effector functions. In some embodiments, the immune effector cells express at least FcyRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils.

In some embodiments, the immune cells are T cells. In some embodiments, the T cells are CD4+/CD8-, CD4-/CD8+, CD4+/CD8+, CD4-/CD8-, or combinations thereof. In some embodiments, the T cells produce IL-2, TFN, and/or TNF upon expressing the chimeric receptor polypeptide and binding to the target cells, such as CD20+ or CD19+ tumor cells. In some embodiments, the CD8+ T cells lyse antigen-specific target cells upon expressing the chimeric receptor polypeptide and binding to the target cells. In some embodiments, the immune cell is selected from the group consisting of a cytotoxic T cell, a helper T cell, a natural killer T (NKT) cell, and a suppressor T cell.

In some embodiments, the immune cells (e.g., immune effector cells) are differentiated from a stem cell, such as a hematopoietic stem cell, a pluripotent stem cell, an iPS, or an embryonic stem cell.

The engineered immune cells (e.g., immune effector cells) are prepared by introducing any of the chimeric receptor polypeptides described herein (and/or a further CAR/engineered TCR) into the immune cells, such as T cells. In some embodiments, the chimeric receptor polypeptide described herein (and/or a further CAR/engineered TCR) is introduced to the immune cells (e.g., immune effector cells such as T cells) by transfecting any one of the isolated nucleic acids or any one of the vectors described herein. In some embodiments, the nucleic acid encoding any of the chimeric receptor polypeptides described herein, and the nucleic acid encoding the CAR/engineered TCR are on separate vectors. In some embodiments, the nucleic acid encoding any of the chimeric receptor polypeptides described herein, and the nucleic acid encoding the CAR/engineered TCR are on the same vector. In some embodiments, the nucleic acid encoding any of the chimeric receptor polypeptides described herein, and the nucleic acid encoding the CAR/engineered TCR are regulated under different promoters. In some embodiments, the nucleic acid encoding any of the chimeric receptor polypeptides described herein, and the nucleic acid encoding the CAR/engineered TCR are regulated under the same promoter. In some embodiments, the chimeric receptor polypeptide described herein (and/or a further CAR/engineered TCR) is introduced to the immune cells (e.g., immune effector cells such as T cells) by inserting proteins into the cell membrane while passing cells through a microfluidic system, such as CELL SQUEEZE® (see, for example, U.S. Pat. Application Publication No. 20140287509).

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

In the cases when a pharmaceutical composition comprises a plurality of engineered mammalian immune cells (e.g., T cell), the engineered mammalian immune cells can be a specific subpopulation of an immune cell type, a combination of subpopulations of an immune cell type, or a combination of two or more immune cell types. In some embodiments, the immune cell is present in a homogenous cell population. In some embodiments, the immune cell is present in a heterogeneous cell population that is enriched in the immune cell. In some embodiments, the engineered mammalian cell is a lymphocyte. In some embodiments, the engineered mammalian cell is not a lymphocyte. In some embodiments, the engineered mammalian cell is suitable for adoptive immunotherapy. In some embodiments, the engineered mammalian cell is a PBMC. In some embodiments, the engineered mammalian cell is an immune cell derived from the PBMC. In some embodiments, the engineered mammalian cell is a T cell. In some embodiments, the engineered mammalian cell is a CD4+ T cell. In some embodiments, the engineered mammalian cell is a CD8+ T cell.

In some embodiments, the immune cell is a T cell. In some embodiments, the immune cell is selected from the group consisting of a cytotoxic T cell, a helper T cell, a natural killer T cell, and a suppressor T cell. In some embodiments, the immune cell is modified to block or decrease the expression of one or both of the endogenous TCR subunits from which the chimeric receptor polypeptides are derived, such as TCRα, TCRβ, TCRγ, TCRδ, CD3ε, CD3γ, and CD3δ. Modification of cells to disrupt gene expression includes any such techniques known in the art, including for example RNA interference (e.g., siRNA, shRNA, miRNA), gene editing (e.g., CRISPR- or TALEN-based gene knockout), and the like.

The immune cell (e.g., T cell) described herein may comprise any number (such as any of 1, 2, 3, 4, 5, 10, 50, 100, 1000, or more) of the nucleic acid encoding a chimeric receptor polypeptide. In some embodiments, the immune cell comprises a single copy of the nucleic acid. In some embodiments, the immune cell comprises a plurality of copies of the nucleic acid. In some embodiments, the immune cell comprises two or more nucleic acids, each encoding a different chimeric receptor polypeptides. The different chimeric receptor polypeptides can be present in the same TCR complex. Alternatively, the different chimeric receptor polypeptides are each incorporated into a different TCR complex.

The nucleic acids described herein can be present in a heterologous gene expression cassette, which comprises one or more protein-coding sequences and optionally one or more promoters. In some embodiments, the heterologous gene expression cassette comprises a single protein-coding sequence. In some embodiments, the heterologous gene expression cassette comprises two or more protein-coding sequences driven by a single promoter (i.e., polycistronic). In some embodiments, the heterologous gene expression cassette further comprises one or more regulatory sequences (such as 5′UTR, 3′UTR, enhancer sequence, IRES, transcription termination sequence), recombination sites, one or more selection markers (such as antibiotic resistance gene, reporter gene, etc.), signal sequence, or combinations thereof. In some embodiments, the nucleic acid encoding the chimeric receptor polypeptide further comprises a coding sequence for a signal sequence for secretion (e.g., signal peptide).

The nucleic acid may be transiently or stably incorporated in the immune cell (e.g., T cell). In some embodiments, the nucleic acid is transiently expressed in the immune cell. For example, the nucleic acid may be present in the nucleus of the immune cell in an extrachromosomal array comprising the heterologous gene expression cassette. Heterologous nucleic acids may be introduced into the immune cell using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnectofection or magnet assisted transfection, particle bombardment; and hybrid methods, such as nucleofection. In some embodiments, the nucleic acid is a DNA. In some embodiments, the nucleic acid is an RNA. In some embodiments, the nucleic acid is linear. In some embodiments, the nucleic acid is circular.

In some embodiments, the nucleic acid described herein is present in the genome of the immune cell (e.g., T cell). For example, the nucleic acid may be integrated into the genome of the immune cell by any methods known in the art, including, but not limited to, virus-mediated integration, random integration, homologous recombination methods, and site-directed integration methods, such as using site-specific recombinase or integrase, transposase, Transcription activator-like effector nuclease (TALEN®), CRISPR/Cas9, and zinc-finger nucleases. In some embodiments, the nucleic acid is integrated in a specifically designed locus of the genome of the immune cell. In some embodiments, the nucleic acid is integrated in an integration hotspot of the genome of the immune cell. In some embodiments, the nucleic acid is integrated in a random locus of the genome of the immune cell. In the cases that multiple copies of the nucleic acids are present in a single immune cell, the nucleic acid may be integrated in a plurality of loci of the genome of the immune cell.

In some embodiments, the immune cell (e.g., T cell) comprises two or more (such as 2, 3, 4, 5, or more) chimeric receptor polypeptides. In some embodiments, the two or more chimeric receptor polypeptides are the same. In some embodiments, the two or more chimeric receptor polypeptides are different from each other. The different chimeric receptor polypeptides can differ from each other in one or more of the following domains: a) the extracellular target binding domain; b) the extracellular TCR binding domain; c) the transmembrane domain; and d) the intracellular domain. The different chimeric receptor polypeptides can also differ from each other in the optional linker connecting the extracellular target binding domain with the extracellular TCR binding domain, the optional linker connecting the extracellular target binding domain (or the extracellular TCR binding domain if at C-terminus of the extracellular target binding domain) with the transmembrane domain, the optionally present extracellular domain of a TCR subunit, or the optionally present hinge region. The two or more chimeric receptor polypeptides can be incorporated into a single TCR complex, or each incorporated into a different TCR complex independently.

For example, in some embodiments, there is provided an immune cell (such as T cell) expressing on its surface any of the chimeric receptor polypeptides described herein. In some embodiments, there is provided an immune cell (such as a T cell) comprising a nucleic acid encoding a chimeric receptor polypeptide according to any of the chimeric receptor polypeptides described herein, wherein the chimeric receptor polypeptide is expressed from the nucleic acid and localized to the immune cell surface.

In some embodiments, there is provide an immune cell (e.g., T cell) expressing on its surface a chimeric receptor polypeptide, wherein the chimeric receptor polypeptide comprises (or consists essentially of or consists of): a) an extracellular target binding domain (e.g., anti-BCMA sdAb); b) an extracellular TCR binding domain (e.g., anti-CD3 scFv or sdAb, or anti-TCR scFv); c) a transmembrane domain, and d) an intracellular domain comprising an intracellular domain of a TCR subunit, wherein the TCR subunit is selected from the group consisting of CD3ε, CD3γ, CD3δ, TCRα, TCRβ, TCRγ, and TCRδ. In some embodiments, there is provide an immune cell (e.g., T cell) comprising a nucleic acid encoding a chimeric receptor polypeptide, wherein the chimeric receptor polypeptide comprises (or consists essentially of or consists of): a) an extracellular target binding domain (e.g., anti-BCMA sdAb); b) an extracellular TCR binding domain (e.g., anti-CD3 scFv or sdAb, or anti-TCR scFv); c) a transmembrane domain, and d) an intracellular domain comprising an intracellular domain of a TCR subunit, wherein the TCR subunit is selected from the group consisting of CD3ε, CD3γ, CD3δ, TCRα, TCRβ, TCRγ, and TCRδ. In some embodiments, the immune cell comprises two or more (such as 2, 3, 4, 5, or more) chimeric receptor polypeptides. In some embodiments, the two or more chimeric receptor polypeptides are different from each other.

In some embodiments, there is provide an immune cell (e.g., T cell) expressing on its surface a chimeric receptor polypeptide, wherein the chimeric receptor polypeptide comprises (or consists essentially of or consists of): a) an extracellular target binding domain (e.g., anti-BCMA sdAb); b) an extracellular TCR binding domain (e.g., anti-CD3 scFv or sdAb, or anti-TCR scFv); and c) transmembrane domain comprising a transmembrane domain of a TCR subunit, wherein the TCR subunit is selected from the group consisting of CD3ε, CD3γ, CD3δ, TCRα, TCRβ, TCRγ, and TCRδ. In some embodiments, there is provide an immune cell (e.g., T cell) comprising a nucleic acid encoding a chimeric receptor polypeptide, wherein the chimeric receptor polypeptide comprises (or consists essentially of or consists of): a) an extracellular target binding domain (e.g., anti-BCMA sdAb); b) an extracellular TCR binding domain (e.g., anti-CD3 scFv or sdAb, or anti-TCR scFv); and c) transmembrane domain comprising a transmembrane domain of a TCR subunit, wherein the TCR subunit is selected from the group consisting of CD3ε, CD3γ, CD3δ, TCRα, TCRβ, TCRγ, and TCRδ. In some embodiments, the immune cell comprises two or more (such as 2, 3, 4, 5, or more) chimeric receptor polypeptides. In some embodiments, the two or more chimeric receptor polypeptides are different from each other.

In some embodiments, there is provided an immune cell (such as T cell) expressing on its surface a chimeric receptor polypeptide, wherein the chimeric receptor polypeptide comprises (or consists essentially of or consists of): a) an extracellular target binding domain (e.g., anti-BCMA sdAb); b) an extracellular TCR binding domain (e.g., anti-CD3 scFv or sdAb, or anti-TCR scFv); and c) transmembrane domain comprising a transmembrane domain of a TCR subunit, wherein the TCR subunit is selected from the group consisting of CD3ε, CD3γ, CD3δ, TCRα, TCRβ, TCRγ, and TCRδ, and d) an intracellular domain. In some embodiments, there is provided an immune cell (such as T cell) comprising a nucleic acid encoding a chimeric receptor polypeptide, wherein the chimeric receptor polypeptide comprises (or consists essentially of or consists of): a) an extracellular target binding domain (e.g., anti-BCMA sdAb); b) an extracellular TCR binding domain (e.g., anti-CD3 scFv or sdAb, or anti-TCR scFv); and c) transmembrane domain comprising a transmembrane domain of a TCR subunit, wherein the TCR subunit is selected from the group consisting of CD3ε, CD3γ, CD3δ, TCRα, TCRβ, TCRγ, and TCRδ, and d) an intracellular domain. In some embodiments, the immune cell comprises two or more (such as 2, 3, 4, 5, or more) chimeric receptor polypeptides. In some embodiments, the two or more chimeric receptor polypeptides are different from each other.

In some embodiments, there is provided an immune cell (such as T cell) expressing on its surface a chimeric receptor polypeptide, wherein the chimeric receptor polypeptide comprises (or consists essentially of or consists of): a) an extracellular target binding domain (e.g., anti-BCMA sdAb); b) an extracellular TCR binding domain (e.g., anti-CD3 scFv or sdAb, or anti-TCR scFv); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided an immune cell (such as T cell) comprising a nucleic acid encoding a chimeric receptor polypeptide, wherein the chimeric receptor polypeptide comprises (or consists essentially of or consists of): a) an extracellular target binding domain (e.g., anti-BCMA sdAb); b) an extracellular TCR binding domain (e.g., anti-CD3 scFv or sdAb, or anti-TCR scFv); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, the immune cell comprises two or more (such as 2, 3, 4, 5, or more) chimeric receptor polypeptides. In some embodiments, the two or more chimeric receptor polypeptides are different from each other. In some embodiments, the first TCR subunit is CD3ε and/or the second TCR subunit is CD3ε. In some embodiments, the first TCR subunit is CD3γ and/or the second TCR subunit is CD3γ. In some embodiments, the first TCR subunit is CD3δ and/or the second TCR subunit is CD3δ. In some embodiments, the first TCR subunit is TCRα and/or the second TCR subunit is TCRα. In some embodiments, the first TCR subunit is TCRβ and/or the second TCR subunit is TCRβ. In some embodiments, the first TCR subunit is TCRγ and/or the second TCR subunit is TCRγ. In some embodiments, the first TCR subunit is TCRδ and/or the second TCR subunit is TCRδ. In some embodiments, the first TCR subunit and the second TCR subunit are the same. In some embodiments, the first TCR subunit and the second TCR subunit are both selected from the group consisting of TCRα, TCRβ, TCRγ, and TCRδ, and the chimeric receptor polypeptide does not comprise a variable region of the extracellular domain of the first TCR subunit or the second TCR subunit. In some embodiments, the chimeric receptor polypeptide does not comprise a variable region of the extracellular domain of any of TCRα, TCRβ, TCRγ, and TCRδ. In some embodiments, the first TCR subunit and the second TCR subunit are different. In some embodiments, the chimeric receptor polypeptide does not comprise an extracellular domain of the first and/or second TCR subunits. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the chimeric receptor polypeptide further comprises a signal peptide at the N-terminus of the chimeric receptor polypeptide. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a hinge region (e.g., CD8 hinge region) adjacent to the transmembrane domain of the chimeric receptor polypeptide on the extracellular region. In some embodiments, the extracellular TCR binding domain comprises two or more TCR antigen binding domains (e.g., scFv or sdAb) arranged in tandem. In some embodiments, the extracellular target binding domain comprises two or more target antigen binding domains (e.g., scFv, sdAb, or DARPin) arranged in tandem. In some embodiments, the immune cell comprises two or more (such as 2, 3, 4, 5, or more) chimeric receptor polypeptides. In some embodiments, the two or more chimeric receptor polypeptides are different from each other. In some embodiments, the chimeric receptor polypeptide comprises a linker between the extracellular target binding domain and the extracellular TCR binding domain. In some embodiments, the chimeric receptor polypeptide comprises a linker between the extracellular target binding domain (or extracellular TCR binding domain) and the transmembrane domain. In some embodiments, the linker is a GS linker, an α-helical linker, a glycine-alanine polymer linker, an alanine-serine polymer linker, or an IgG4-Fc linker. In some embodiments, the linker is selected from the group consisting of SEQ ID NOs: 1-21, 67, and 68.

In some embodiments, there is provided an immune cell (such as T cell) expressing on its surface a chimeric receptor polypeptide, wherein the chimeric receptor polypeptide comprises (or consists essentially of or consists of): a) an extracellular target binding domain comprising a target antigen binding domain (such as scFv, sdAb, DARPin) specifically binding to a target antigen (e.g., BCMA); b) an extracellular TCR binding domain comprising a TCR antigen binding domain (such as scFv or sdAb) specifically binding to a TCR subunit (e.g., extracellular domain of a TCR subunit, such as extracellular domain of CD3ε or TCR); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, there is provided an immune cell (such as T cell) comprising a nucleic acid encoding a chimeric receptor polypeptide, wherein the chimeric receptor polypeptide comprises (or consists essentially of or consists of): a) an extracellular target binding domain comprising a target antigen binding domain (such as scFv, sdAb, DARPin) specifically binding to a target antigen (e.g., BCMA); b) an extracellular TCR binding domain comprising a TCR antigen binding domain (such as scFv or sdAb) specifically binding to a TCR subunit (e.g., extracellular domain of a TCR subunit, such as extracellular domain of CD3ε or TCR); c) a transmembrane domain comprising a transmembrane domain of a first TCR subunit; and d) an intracellular domain comprising an intracellular domain of a second TCR subunit, wherein the first TCR subunit and the second TCR subunit are both selected from the group consisting of CD3ε, CD3γ, CD3δ, TCRα, TCRβ, TCRγ, and TCRδ. In some embodiments, the immune cell comprises two or more (such as 2, 3, 4, 5, or more) chimeric receptor polypeptides. In some embodiments, the two or more chimeric receptor polypeptides are different from each other. In some embodiments, the target antigen binding domain is an sdAb specifically binding to BCMA, such as any of the anti-BCMA sdAbs disclosed in PCT/CN2016/094408 and PCT/CN2017/096938. In some embodiments, the anti-BCMA sdAb comprises CDR1, CDR2, and CDR3 of the amino acid sequence of any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the anti-BCMA sdAb comprises CDR1, CDR2, and CDR3 of the amino acid sequence of SEQ ID NO: 26. In some embodiments, the chimeric receptor polypeptide comprises a linker between the extracellular target binding domain and the extracellular TCR binding domain. In some embodiments, the chimeric receptor polypeptide comprises a linker between the extracellular target binding domain (or extracellular TCR binding domain) and the transmembrane domain. In some embodiments, the linker is a GS linker, an α-helical linker, a glycine-alanine polymer linker, an alanine-serine polymer linker, or an IgG4-Fc linker. In some embodiments, the linker is selected from the group consisting of SEQ ID NOs: 1-21, 67, and 68.

In some embodiments, there is provided an immune cell (such as T cell) expressing on its surface a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of CD3ε; and d) an intracellular domain comprising an intracellular domain of CD3ε. In some embodiments, there is provided an immune cell (such as T cell) comprising a nucleic acid encoding a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of CD3ε; and d) an intracellular domain comprising an intracellular domain of CD3ε. In some embodiments, the chimeric receptor polypeptide does not comprise an extracellular domain of CD3ε. In some embodiments, the chimeric receptor polypeptide does not comprise an extracellular domain of any TCR subunits. In some embodiments, there is provided an immune cell (such as T cell) expressing on its surface a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of CD3γ; and d) an intracellular domain comprising an intracellular domain of CD3γ. In some embodiments, there is provided an immune cell (such as T cell) comprising a nucleic acid encoding a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising an antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of CD3γ; and d) an intracellular domain comprising an intracellular domain of CD3γ. In some embodiments, the chimeric receptor polypeptide does not comprise an extracellular domain of CD3γ. In some embodiments, the chimeric receptor polypeptide does not comprise an extracellular domain of any TCR subunits. In some embodiments, there is provided an immune cell (such as T cell) expressing on its surface a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of CD3δ; and d) an intracellular domain comprising an intracellular domain of CD3δ. In some embodiments, there is provided an immune cell (such as T cell) comprising a nucleic acid encoding a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of CD3δ; and d) an intracellular domain comprising an intracellular domain of CD3δ. In some embodiments, the chimeric receptor polypeptide does not comprise an extracellular domain of CD3δ. In some embodiments, the chimeric receptor polypeptide does not comprise an extracellular domain of any TCR subunits. In some embodiments, there is provided an immune cell (such as T cell) expressing on its surface a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of TCRα; and d) an intracellular domain comprising an intracellular domain of TCRα. In some embodiments, there is provided an immune cell (such as T cell) comprising a nucleic acid encoding a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of TCRα; and d) an intracellular domain comprising an intracellular domain of TCRα. In some embodiments, the chimeric receptor polypeptide does not comprise variable region of the extracellular domain of TCRα. In some embodiments, the chimeric receptor polypeptide does not comprise an extracellular domain of TCRα. In some embodiments, the chimeric receptor polypeptide does not comprise an extracellular domain of any TCR subunits. In some embodiments, there is provided an immune cell (such as T cell) expressing on its surface a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of TCRβ; and d) an intracellular domain comprising an intracellular domain of TCRβ. In some embodiments, there is provided an immune cell (such as T cell) comprising a nucleic acid encoding a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of TCRβ; and d) an intracellular domain comprising an intracellular domain of TCRβ. In some embodiments, the chimeric receptor polypeptide does not comprise variable region of the extracellular domain of TCRβ. In some embodiments, the chimeric receptor polypeptide does not comprise an extracellular domain of TCRβ. In some embodiments, the chimeric receptor polypeptide does not comprise an extracellular domain of any TCR subunits. In some embodiments, there is provided an immune cell (such as T cell) expressing on its surface a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of TCRy; and d) an intracellular domain comprising an intracellular domain of TCRγ. In some embodiments, there is provided an immune cell (such as T cell) comprising a nucleic acid encoding a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of TCRγ; and d) an intracellular domain comprising an intracellular domain of TCRγ. In some embodiments, the chimeric receptor polypeptide does not comprise variable region of the extracellular domain of TCRγ. In some embodiments, the chimeric receptor polypeptide does not comprise an extracellular domain of TCRγ. In some embodiments, the chimeric receptor polypeptide does not comprise an extracellular domain of any TCR subunits. In some embodiments, there is provided an immune cell (such as T cell) expressing on its surface a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of TCRδ; and d) an intracellular domain comprising an intracellular domain of TCRδ. In some embodiments, there is provided an immune cell (such as T cell) comprising a nucleic acid encoding a chimeric receptor polypeptide, wherein the chimeric receptor polypeptide comprises (or consists essentially of or consists of): a) an extracellular target binding domain (for example an scFv, sdAb, DARPin recognizing a target antigen such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of TCRδ; and d) an intracellular domain comprising an intracellular domain of TCRδ. In some embodiments, the chimeric receptor polypeptide does not comprise variable region of the extracellular domain of TCRδ. In some embodiments, the chimeric receptor polypeptide does not comprise an extracellular domain of TCRδ. In some embodiments, the chimeric receptor polypeptide does not comprise an extracellular domain of any TCR subunits. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the immune cell comprises two or more (such as 2, 3, 4, 5, or more) chimeric receptor polypeptides. In some embodiments, the two or more chimeric receptor polypeptides are different from each other. In some embodiments, the target antigen binding domain is an sdAb specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the anti-BCMA sdAb comprises CDR1, CDR2, and CDR3 of the amino acid sequence of SEQ ID NO: 26. In some embodiments, the TCR antigen binding domain (e.g., scFv, sdAb) specifically binds to CD3ε (e.g., N-terminus of CD3s). In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-CD3 sdAb comprising CDR1, CDR2, and CDR3 of the amino acid sequence of SEQ ID NO: 22 or 23. In some embodiments, the TCR antigen binding domain is an anti-CD3 scFv comprising HC-CDR 1-3 and LC-CDR 1-3 of the amino acid sequence of SEQ ID NO: 24. In some embodiments, the TCR antigen binding domain (e.g., scFv, sdAb) specifically binds to TCRγ/δ (e.g., constant region of TCRγ/δ). In some embodiments, the TCR antigen binding domain is an anti-TCR scFv comprising the amino acid sequence of SEQ ID NO: 27. In some embodiments, the TCR antigen binding domain is an anti-TCR scFv comprising HC-CDR 1-3 and LC-CDR 1-3 of the amino acid sequence of SEQ ID NO: 27. In some embodiments, the extracellular TCR binding domain comprises two or more TCR antigen binding domains (e.g., scFv or sdAb) arranged in tandem. In some embodiments, the extracellular target binding domain comprises two or more target antigen binding domains (e.g., scFv, sdAb, or DARPin) arranged in tandem. In some embodiments, the chimeric receptor polypeptide further comprises a hinge region (e.g., CD8 hinge region) adjacent to the transmembrane domain of the chimeric receptor polypeptide at the extracellular region. In some embodiments, the chimeric receptor polypeptide comprises a linker between the extracellular target binding domain and the extracellular TCR binding domain. In some embodiments, the chimeric receptor polypeptide comprises a linker between the extracellular target binding domain (or extracellular TCR binding domain) and the transmembrane domain. In some embodiments, the linker is a GS linker, an α-helical linker, a glycine-alanine polymer linker, an alanine-serine polymer linker, or an IgG4-Fc linker. In some embodiments, the linker is selected from the group consisting of SEQ ID NOs: 1-21, 67, and 68.

In some embodiments, there is provided an immune cell expressing on its surface a chimeric receptor polypeptide, wherein the chimeric receptor polypeptide comprises (or consists essentially of or consists of): a) an extracellular target binding domain comprising two or more (such as two) target antigen binding domains (such as scFv, sdAb, DARPin), each specifically binding to a target antigen (such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of CD3ε (or CD3γ or CD3δ); and d) an intracellular domain comprising an intracellular domain of CD3ε (or CD3γ or CD3δ). In some embodiments, there is provided an immune cell comprising a nucleic acid encoding a chimeric receptor polypeptide, wherein the chimeric receptor polypeptide comprises (or consists essentially of or consists of): a) an extracellular target binding domain comprising two or more (such as two) target antigen binding domains (such as scFv, sdAb, DARPin), each specifically binding to a target antigen (such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of CD3ε (or CD3γ or CD3δ); and d) an intracellular domain comprising an intracellular domain of CD3ε (or CD3γ or CD3δ). In some embodiments, the chimeric receptor polypeptide does not comprise an extracellular domain of any TCR subunits. In some embodiments, the chimeric receptor polypeptide does not comprise an extracellular domain of CD3ε, CD3γ, or CD3δ. In some embodiments, there is provided an immune cell expressing on its surface a chimeric receptor polypeptide, wherein the chimeric receptor polypeptide comprises (or consists essentially of or consists of): a) an extracellular target binding domain comprising two or more (such as two) target antigen binding domains (such as scFv, sdAb, DARPin), each specifically binding to a target antigen (such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of TCRα (or TCRβ or TCRγ or TCRδ); and d) an intracellular domain comprising an intracellular domain of TCRα (or TCRβ or TCRγ or TCRδ). In some embodiments, there is provided an immune cell comprising a nucleic acid encoding a chimeric receptor polypeptide, wherein the chimeric receptor polypeptide comprises (or consists essentially of or consists of): a) an extracellular target binding domain comprising two or more (such as two) target antigen binding domains (such as scFv, sdAb, DARPin), each specifically binding to a target antigen (such as BCMA); b) an extracellular TCR binding domain comprising a TCR antigen binding domain (such as scFv or sdAb) specifically binding to CD3 (such as CD3ε, for example the N-terminus of CD3ε) or TCR (such as TCRγ/δ, for example the constant region of TCRγ/δ); c) a transmembrane domain comprising a transmembrane domain of TCRα (or TCRβ or TCRγ or TCRδ); and d) an intracellular domain comprising an intracellular domain of TCRα (or TCRβ or TCRγ or TCRδ). In some embodiments, the chimeric receptor polypeptide does not comprise the variable region of the extracellular domain of TCRα, TCRβ, TCRγ, or TCRδ. In some embodiments, the chimeric receptor polypeptide does not comprise an extracellular domain of TCRα, TCRβ, TCRγ, or TCRδ. In some embodiments, the chimeric receptor polypeptide does not comprise an extracellular domain of any TCR subunits. In some embodiments, the two or more target antigen binding domains bind to the same antigen or the same epitope on an antigen. In some embodiments, the two or more target antigen binding domains bind to different epitopes on the same antigen (such as BCMA). In some embodiments, the two or more target antigen binding domains bind to a different antigen. In some embodiments, the two or more target antigen binding domains are the same. In some embodiments, the two or more target antigen binding domains are different. In some embodiments, the chimeric receptor polypeptide does not comprise an intracellular co-stimulatory domain. In some embodiments, the extracellular target binding domain is N-terminal to the extracellular TCR binding domain. In some embodiments, the extracellular target binding domain is C-terminal to the extracellular TCR binding domain. In some embodiments, the chimeric receptor polypeptide further comprises a signal peptide at the N-terminus of the chimeric receptor polypeptide. In some embodiments, the chimeric receptor polypeptide comprises a linker between the extracellular target binding domain and the extracellular TCR binding domain. In some embodiments, the chimeric receptor polypeptide comprises a linker between the extracellular target binding domain (or extracellular TCR binding domain) and the transmembrane domain. In some embodiments, two or more target antigen binding domains (e.g., scFv, sdAb, or DARPin) are arranged in tandem. In some embodiments, the chimeric receptor polypeptide comprises a linker between the two or more target antigen binding domains (e.g., scFv, sdAb, DARPin) within the extracellular target binding domain. In some embodiments, the linker is a GS linker, an α-helical linker, a glycine-alanine polymer linker, an alanine-serine polymer linker, or an IgG4-Fc linker. In some embodiments, the linker is selected from the group consisting of SEQ ID NOs: 1-21, 67, and 68. In some embodiments, the extracellular TCR binding domain comprises two or more TCR antigen binding domains (e.g., scFv or sdAb) arranged in tandem. In some embodiments, the chimeric receptor polypeptide further comprises a hinge region (e.g., CD8 hinge region) adjacent to the transmembrane domain of the chimeric receptor polypeptide on the extracellular side. In some embodiments, the immune cell comprises two or more (such as 2, 3, 4, 5, or more) chimeric receptor polypeptides. In some embodiments, the two or more chimeric receptor polypeptides are different from each other. In some embodiments, the target antigen binding domain is an sdAb specifically binding to BCMA, such as any of the anti-BCMA sdAbs disclosed in PCT/CN2016/094408 and PCT/CN2017/096938. In some embodiments, the anti-BCMA sdAb comprises the amino acid sequence of SEQ ID NO: 26. In some embodiments, the anti-BCMA sdAb comprises CDR1, CDR2, and CDR3 of the amino acid sequence of SEQ ID NO: 26. In some embodiments, the TCR antigen binding domain (e.g., scFv, sdAb) specifically binds to CD3ε (e.g., N-terminus of CD3s). In some embodiments, the TCR antigen binding domain is an anti-CD3 sdAb comprising the amino acid sequence of SEQ ID NO: 22 or 23. In some embodiments, the TCR antigen binding domain is an anti-CD3 scFv comprising the amino acid sequence of SEQ ID NO: 24. In some embodiments, the TCR antigen binding domain is an anti-CD3 sdAb comprising CDR1, CDR2, and CDR3 of the amino acid sequence of SEQ ID NO: 22 or 23. In some embodiments, the TCR antigen binding domain is an anti-CD3 scFv comprising HC-CDR 1-3 and LC-CDR 1-3 of the amino acid sequence of SEQ ID NO: 24. In some embodiments, the TCR antigen binding domain (e.g., scFv, sdAb) specifically binds to TCRγ/δ (e.g., constant region of TCRγ/δ). In some embodiments, the TCR antigen binding domain is an anti-TCR scFv comprising the amino acid sequence of SEQ ID NO: 27. In some embodiments, the TCR antigen binding domain is an anti-TCR scFv comprising HC-CDR 1-3 and LC-CDR 1-3 of the amino acid sequence of SEQ ID NO: 27.

### Preparation of Immune Cells Expressing Chimeric Receptor Polypeptides

The present invention in one aspect provides immune cells (such as lymphocytes, for example T cells) expressing a chimeric receptor polypeptide described herein. Exemplary methods of preparing immune cells (such as T cells) expressing the chimeric receptor polypeptides are provided herein.

In some embodiments, an immune cell expressing the chimeric receptor polypeptide can be generated by introducing one or more nucleic acids (including for example a lentiviral vector) encoding a chimeric receptor polypeptide (such as any of the chimeric receptor polypeptides described herein) that specifically binds to a target antigen (such as a disease-associated antigen) into the immune cell. The introduction of the one or more nucleic acids into the immune cell can be accomplished using techniques known in the art. In some embodiments, the immune cells (such as T cells) of the invention are able to replicate in vivo, resulting in long-term persistence that can lead to sustained control of a disease associated with expression of the target antigen (such as cancer, autoimmune disease, or viral infection).

In some embodiments, the invention relates to administering a genetically modified immune cell (such as T cell) expressing a chimeric receptor polypeptide that specifically binds to a target antigen according to any of the chimeric receptor polypeptides described herein for the treatment of a patient having or at risk of developing a disease and/or disorder associated with expression of the target antigen (also referred to herein as a “target antigen-positive” or “TA-positive” disease or disorder), including, for example, cancer or viral infection, using lymphocyte infusion. In some embodiments, autologous lymphocyte infusion is used in the treatment. Autologous PBMCs are collected from a patient in need of treatment and T cells are activated and expanded using the methods described herein and known in the art and then infused back into the patient.

In some embodiments, there is provided a T cell expressing a chimeric receptor polypeptide that specifically binds to a target antigen (e.g., BCMA) according to any of the chimeric receptor polypeptides described herein (also referred to herein as an “engineered T cell”). The engineered T cells of the invention can undergo robust in vivo T cell expansion and can establish target antigen-specific memory cells that persist at high levels for an extended amount of time in blood and bone marrow. In some embodiments, the engineered T cells of the invention infused into a patient can eliminate target antigen-presenting cells, such as target antigen-presenting cancer or virally-infected cells, in vivo in patients having a target antigen-associated disease. In some embodiments, the engineered T cells of the invention infused into a patient can eliminate target antigen-presenting cells, such as target antigen-presenting cancer or virally-infected cells, in vivo in patients having a target antigen-associated disease that is refractory to at least one conventional treatment.

Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments of the present invention, any number of T cell lines available in the art may be used. In some embodiments of the present invention, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer’s instructions. After washing, the cells may be resuspended in a variety of biocompatible buffers, such as Ca²⁺-free, Mg²⁺-free PBS, PlasmaLyte A, or other saline solutions with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such as in isolating tumor infiltrating lymphocytes (TIL) from tumor tissue or from immune-compromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD11b, CD 16, HLA-DR, and CD8. In some embodiments, it may be desirable to enrich for or positively select for regulatory T cells which typically express CD4⁺, CD25⁺, CD62Lhi, GITR⁺, and FoxP3⁺. Alternatively, in some embodiments, T regulatory cells are depleted by anti-CD25 conjugated beads or other similar methods of selection.

For isolation of a desired population of cells by positive or negative selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In some embodiments of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in some embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Whether prior to or after genetic modification of the T cells to express a desirable chimeric receptor polypeptide, the T cells can be activated and expanded generally using methods as described, for example, in U.S. Pat. Nos. 6,352,694.

In some embodiments, the immune cells (such as T cells) expressing the chimeric receptor polypeptides of the invention are generated by transducing immune cells (such as T cells prepared by the methods described herein) with a viral vector encoding a chimeric receptor polypeptide as described herein. Viral vector delivery systems include DNA and RNA viruses, which have either episomal or integrated genomes after delivery to the immune cell. In some embodiments, the viral vector is a lentiviral vector, and the immune cell comprises the lentiviral vector integrated into the immune cell genome.

### Enrichment

In some embodiments, there is provided a method of enriching a heterogeneous cell population for an immune cell (e.g., T cell) expressing the chimeric receptor polypeptide according to any of the modified/engineered immune cells described herein.

A specific subpopulation of immune cells (such as T cells) expressing the chimeric receptor polypeptides that specifically bind to a target antigen (e.g., BCMA) can be enriched by positive selection techniques. For example, in some embodiments, engineered immune cells (such as T cells) are enriched for by incubation with target antigen-conjugated beads for a time period sufficient for positive selection of the desired engineered immune cells. For isolation of modified immune cells present at low levels in the heterogeneous cell population, use of longer incubation times, such as 24 hours, can increase cell yield. The skilled artisan would recognize that multiple rounds of selection can also be used in the context of this invention.

For isolation of a desired population of modified immune cells by positive selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads.

In some of any such embodiments described herein, enrichment results in minimal or substantially no exhaustion of the modified immune cells. For example, in some embodiments, enrichment results in fewer than about 50% (such as fewer than about any of 45, 40, 35, 30, 25, 20, 15, 10, or 5%) of the modified immune cells becoming exhausted. Immune cell exhaustion can be determined by any means known in the art, including any means described herein.

In some of any such embodiments described herein, enrichment results in minimal or substantially no terminal differentiation of the modified immune cells. For example, in some embodiments, enrichment results in fewer than about 50% (such as fewer than about any of 45, 40, 35, 30, 25, 20, 15, 10, or 5%) of the modified immune cells becoming terminally differentiated. Immune cell differentiation can be determined by any means known in the art, including any means described herein.

In some of any such embodiments described herein, enrichment results in minimal or substantially no internalization of the chimeric receptor polypeptide on the modified immune cells. For example, in some embodiments, enrichment results in less than about 50% (such as less than about any of 45, 40, 35, 30, 25, 20, 15, 10, or 5%) of chimeric receptor polypeptide on the modified immune cells becoming internalized. Internalization of chimeric receptor polypeptide on the modified immune cells can be determined by any means known in the art, including any means described herein.

Thus, in some embodiments, there is provided a method of enriching a heterogeneous cell population for immune cells (e.g., T cell) expressing a chimeric receptor polypeptide that specifically binds to a target antigen (e.g., BCMA), comprising: a) contacting the heterogeneous cell population with a ligand comprising the target antigen or one or more epitopes contained therein to form complexes comprising the immune cell bound to the ligand; and b) separating the complexes from the heterogeneous cell population, thereby generating a cell population enriched for the immune cells expressing the chimeric receptor polypeptide. In some embodiments, the ligand is immobilized to a solid support. In some embodiments, the solid support is particulate (such as beads). In some embodiments, the solid support is a surface (such as the bottom of a well). In some embodiments, the ligand is labelled with a tag. In some embodiments, the tag is a fluorescent molecule, an affinity tag, or a magnetic tag. In some embodiments, the method further comprises eluting the immune cells from the ligand and recovering the eluate.

### Library Screening

To identify candidate chimeric receptor polypeptides specific for a target antigen, a chimeric receptor polypeptide library, for example cells expressing a library of nucleic acids encoding a plurality of chimeric receptor polypeptides, may be exposed to a ligand comprising the target antigen or one or more epitopes contained therein, followed by isolation of affinity members of the library that specifically bind the ligand. In some embodiments, the ligand is immobilized on a solid support. In some embodiments, the support may be the surfaces of beads, microtitre plates, immunotubes, or any material known in the art useful for such purposes. In some embodiments, the interaction takes place in solution on tagged ligand targets (e.g. biotinylated ligand). In some embodiments, the procedure involves one or more washing steps to remove unspecific and non-reactive library members (panning). In some embodiments, to purify complexes in solution, they are captured by either immobilization or by centrifugation. In some embodiments, affinity members are captured on a soluble biotinylated ligand, followed by immobilization of the affinity complex (affinity member and ligand) on streptavidin beads. In some embodiments, the solid support is a bead. In some embodiments, the beads include, for example, magnetic beads, nonmagnetic beads, monodisperse beads, and polydisperse beads. In some embodiments, the affinity members are purified by positive selection. In some embodiments, the affinity members are purified by negative selection to remove unwanted library members. In some embodiments, the affinity members are purified by both positive and negative selection steps.

Generally, the techniques used to prepare the library constructs will be based on known genetic engineering techniques. In this regard, nucleic acid sequences encoding the chimeric receptor polypeptide to be expressed in the library are incorporated into expression vectors appropriate for the type of expression system to be used. Appropriate expression vectors for use in display in cells, such as CD3⁺ cells, are well known and described in the art. For example, in some embodiments, the expression vector is a viral vector, such as a lentiviral vector.

In some embodiments, there is provided a nucleic acid library comprising sequences encoding a plurality of chimeric receptor polypeptides according to any one of the embodiments described herein. In some embodiments, the nucleic acid library comprises viral vectors encoding the plurality of chimeric receptor polypeptides. In some embodiments, the viral vectors are lentiviral vectors.

In some embodiments, there is provided a method of screening a nucleic acid library according to any of the embodiments described herein for sequences encoding chimeric receptor polypeptides specific for a target antigen, comprising: a) introducing the nucleic acid library into a plurality of cells, such that the chimeric receptor polypeptides are expressed on the surface of the plurality of cells; b) incubating the plurality of cells with a ligand comprising the target antigen or one or more epitopes contained therein; c) collecting cells bound to the ligand; and d) isolating sequences encoding the chimeric receptor polypeptides from cells collected in step c), thereby identifying chimeric receptor polypeptides specific for the target antigen. In some embodiments, the method further comprises one or more wash steps. In some embodiments, the one or more wash steps are carried out between steps b) and c). In some embodiments, the plurality of cells is a plurality of CD3⁺ cells. In some embodiments, the plurality of cells is a plurality of TCR⁺ cells. In some embodiments, the ligand is immobilized on a solid support. In some embodiments, the solid support is a bead. In some embodiments, collecting cells bound to the ligand comprises eluting cells from the ligand bound to the solid support and collecting the eluate. In some embodiments, the ligand is labelled with a tag. In some embodiments, the tag is a fluorescent molecule, an affinity tag, or a magnetic tag. In some embodiments, collecting cells bound to the ligand comprises isolating complexes comprising the cells and the labelled ligand. In some embodiments, the cells are dissociated from the complexes.

### Pharmaceutical Compositions

Also provided herein are compositions (such as pharmaceutical compositions, also referred to herein as formulations) comprising chimeric receptor polypeptide according to any of the embodiments described herein, a nucleic acid (or vector thereof) encoding a chimeric receptor polypeptide according to any of the embodiments described herein, or an immune cell (e.g., T cell) expressing a chimeric receptor polypeptide according to any of the embodiments described herein, and optionally a pharmaceutically acceptable excipient. In some embodiments, the composition is an immune cell composition (such as a pharmaceutical composition) comprising an immune cell (such as a T cell) presenting on its surface a chimeric receptor polypeptide according to any of the chimeric receptor polypeptides described herein.

The composition may comprise a homogenous cell population comprising immune cells of the same cell type and expressing the same chimeric receptor polypeptide, or a heterogeneous cell population comprising a plurality of immune cell populations comprising immune cells of different cell types and/or expressing different chimeric receptor polypeptides. The composition may further comprise cells that are not immune cells.

Thus, in some embodiments, there is provided an immune cell composition comprising a homogeneous cell population of immune cells (such as T cells) of the same cell type and expressing the same chimeric receptor polypeptides. In some embodiments, the immune cell is a T cell. In some embodiments, the immune cell is selected from the group consisting of a cytotoxic T cell, a helper T cell, a natural killer T cell, and a suppressor T cell. In some embodiments, the immune cell composition is a pharmaceutical composition.

In some embodiments, there is provided an immune cell composition comprising a heterogeneous cell population comprising a plurality of immune cell populations comprising immune cells of different cell types and/or expressing different chimeric receptor polypeptides. In some embodiments, the immune cells are T cells. In some embodiments, each population of immune cells is of a cell type selected from the group consisting of cytotoxic T cells, helper T cells, natural killer T cells, and suppressor T cells. In some embodiments, all of the immune cells in the composition are of the same cell type (e.g., all of the immune cells are cytotoxic T cells). In some embodiments, at least one population of immune cells is of a different cell type than the others (e.g., one population of immune cells consists of cytotoxic T cells and the other populations of immune cells consist of natural killer T cells). In some embodiments, each population of the immune cells expresses the same chimeric receptor polypeptides. In some embodiments, at least one population of immune cells expresses a different chimeric receptor polypeptide than the others. In some embodiments, each population of immune cells expresses different chimeric receptor polypeptides than the others. In some embodiments, each population of immune cells expresses a chimeric receptor polypeptide that specifically binds to the same target antigen. In some embodiments, at least one population of immune cells expresses a chimeric receptor polypeptide that specifically binds to a different target antigen (or epitope) than the others. In some embodiments, where at least one population of immune cells expresses aa chimeric receptor polypeptide that specifically binds to a different target antigen (or epitope), each population of immune cells expresses a chimeric receptor polypeptide that specifically binds to a target antigen associated with the same disease or disorder (e.g., each of the target antigens are associated with a cancer, such as breast cancer). In some embodiments, the immune cell composition is a pharmaceutical composition.

Thus, in some embodiments, there is provided an immune cell composition comprising a plurality of immune cell populations according to any of the embodiments described herein, wherein all of the immune cells in the composition are of the same cell type (e.g., all of the immune cells are cytotoxic T cells), and wherein each population of immune cells expresses a different chimeric receptor polypeptide than the others. In some embodiments, the immune cells are T cells. In some embodiments, the immune cells are selected from the group consisting of cytotoxic T cells, helper T cells, natural killer T cells, and suppressor T cells. In some embodiments, each population of immune cells expresses a chimeric receptor polypeptide that specifically binds to the same target antigen. In some embodiments, at least one population of immune cells expresses a chimeric receptor polypeptide that specifically binds to a different target antigen (or epitope) than the others. In some embodiments, where at least one population of the immune cells expresses a chimeric receptor polypeptide that specifically binds to a different target antigen, each population of immune cells expresses a chimeric receptor polypeptide that specifically binds to a target antigen associated with the same disease or disorder (e.g., each of the target antigens are associated with a cancer, such as breast cancer). In some embodiments, the immune cell composition is an immune cell pharmaceutical composition.

In some embodiments, there is provided a composition comprising a plurality of immune cell populations according to any of the embodiments described herein, wherein at least one population of the immune cells is of a different cell type than the others. In some embodiments, all of the populations of the immune cells are of different cell types. In some embodiments, each population of the immune cells is of a cell type selected from the group consisting of cytotoxic T cells, helper T cells, natural killer T cells, and suppressor T cells. In some embodiments, each population of immune cells expresses the same chimeric receptor polypeptide. In some embodiments, at least one population of the immune cells expresses a different chimeric receptor polypeptide than the others. In some embodiments, each population of immune cells expresses a different chimeric receptor polypeptide than the others. In some embodiments, each population of immune cells expresses a chimeric receptor polypeptide that specifically binds to the same target antigen. In some embodiments, at least one population of the immune cells expresses a chimeric receptor polypeptide that specifically binds to a different target antigen (or epitope) than the others. In some embodiments, where at least one population of the immune cells expresses a chimeric receptor polypeptide that specifically binds to a different target antigen (or epitope), each population of the immune cells expresses a chimeric receptor polypeptide that specifically binds to a target antigen associated with the same disease or disorder (e.g., each of the target antigens are associated with a cancer, such as breast cancer). In some embodiments, the immune cell composition is a pharmaceutical composition.

At various points during preparation of a composition, it can be necessary or beneficial to cryopreserve a cell. The terms “frozen/freezing” and “cryopreserved/cryopreserving” can be used interchangeably. Freezing includes freeze drying.

Damage to the cells subject to cryopreservation can be avoided by (a) use of a cryoprotective agent, (b) control of the freezing rate, and/or (c) storage at a temperature sufficiently low to minimize degradative reactions. Exemplary cryoprotective agents include dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, choline chloride, amino acids, methanol, acetamide, glycerol monoacetate, and inorganic salts. In particular embodiments, DMSO can be used. Addition of plasma (e.g., to a concentration of 20-25%) can augment the protective effects of DMSO. After addition of DMSO, cells can be kept at 0° C. until freezing, because DMSO concentrations of 1% can be toxic at temperatures above 4° C.

In the cryopreservation of cells, slow controlled cooling rates can be critical and different cryoprotective agents. The heat of fusion phase where water turns to ice should be minimal. The cooling procedure can be carried out by use of, e.g., a programmable freezing device or a methanol bath procedure. Programmable freezing apparatuses allow determination of optimal cooling rates and facilitate standard reproducible cooling.

In particular embodiments, DMSO-treated cells can be pre-cooled on ice and transferred to a tray containing chilled methanol which is placed, in turn, in a mechanical refrigerator (e.g., Harris or Revco) at -80° C. Thermocouple measurements of the methanol bath and the samples indicate a cooling rate of 1° to 3° C./minute can be preferred. After at least two hours, the specimens can have reached a temperature of - 80° C. and can be placed directly into liquid nitrogen (-196° C.).

After thorough freezing, the cells can be rapidly transferred to a long-term cryogenic storage vessel. In a preferred embodiment, samples can be cryogenically stored in liquid nitrogen (-196° C.) or vapor (-1° C.). Such storage is facilitated by the availability of highly efficient liquid nitrogen refrigerators.

Following cryopreservation, frozen cells can be thawed for use in accordance with methods known to those of ordinary skill in the art. Frozen cells are preferably thawed quickly and chilled immediately upon thawing. In particular embodiments, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed on ice.

In particular embodiments, methods can be used to prevent cellular clumping during thawing. Exemplary methods include: the addition before and/or after freezing of DNase, low molecular weight dextran and citrate, hydroxyethyl starch. As is understood by one of ordinary skill in the art, if a cryoprotective agent that is toxic to humans is used, it should be removed prior to therapeutic use. DMSO has no serious toxicity.

Exemplary carriers and modes of administration of cells are described at pages 14-15 of U.S. Pat. Publication No. 2010/0183564. Additional pharmaceutical carriers are described in Remington: The Science and Practice of Pharmacy, 21 st Edition, David B. Troy, ed., Lippicott Williams & Wilkins (2005).

In particular embodiments, cells can be harvested from a culture medium, and washed and concentrated into a carrier in a therapeutically-effective amount. Exemplary carriers include saline, buffered saline, physiological saline, water, Hanks’ solution, Ringer’s solution, Nonnosol-R (Abbott Labs), Plasma-Lyte A(R) (Baxter Laboratories, Inc., Morton Grove, IL), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2- phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, compositions can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within compositions can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In compositions and formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

Also provided herein are nucleic acid compositions (such as pharmaceutical compositions, also referred to herein as formulations) comprising any of the nucleic acids encoding a chimeric receptor polypeptide described herein. In some embodiments, the nucleic acid composition is a pharmaceutical composition. In some embodiments, the nucleic acid composition further comprises any of an isotonizing agent, an excipient, a diluent, a thickener, a stabilizer, a buffer, and/or a preservative; and/or an aqueous vehicle, such as purified water, an aqueous sugar solution, a buffer solution, physiological saline, an aqueous polymer solution, or RNase free water.

The compositions and formulations disclosed herein can be prepared for administration by, for example, injection, infusion, perfusion, or lavage. The compositions and formulations can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection.

The formulations to be used for in vivo administration must be sterile. This is readily accomplished by, e.g., filtration through sterile filtration membranes.

### Methods of Treatment Using Cells Expressing the Chimeric Receptor Polypeptide

The chimeric receptor polypeptides and/or compositions (such as pharmaceutical compositions) of the invention can be used to treat a disease and/or disorder associated with target antigen (TA) expression (also referred to herein as a “target-antigen positive” or “TA-positive” disease or disorder), including, for example, cancer, infectious disease (such as viral infection), and autoimmune disease. The present application thus in some embodiments provides a method for treating a target antigen-positive disease (such as cancer, viral infection, autoimmune disease) in an individual (e.g., human) comprising administering to the individual an effective amount of an immune cell composition (such as a pharmaceutical composition) expressing a chimeric receptor polypeptide described herein. In some embodiments, the cancer is selected, for example, from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer and thyroid cancer. In some embodiments, the cancer is selected from the group consisting of acute leukemias (including but not limited to acute myeloid leukemia (AML), B-cell acute lymphoid leukemia (BALL), T-cell acute lymphoid leukemia (TALL), and acute lymphoid leukemia (ALL)), chronic leukemias (including but not limited to chronic myelogenous leukemia (CML) and chronic lymphocytic leukemia (CLL)), multiple myeloma (MM), myelodysplastic syndrome (MDS), myeloproliferative neoplasms (MPNs), chronic myeloid leukemia (CML), and blastic plasmacytoid dendritic cell neoplasm (BPDCN). In some embodiments, the viral infection is caused by a virus selected, for example, from the group consisting of Cytomegalovirus (CMV), Epstein-Barr Virus (EBV), Hepatitis B Virus (HBV), Kaposi’s Sarcoma associated herpesvirus (KSHV), Human papillomavirus (HPV), Molluscum contagiosum virus (MCV), Human T cell leukemia virus 1 (HTLV-1), HIV (Human immunodeficiency virus), and Hepatitis C Virus (HCV). In some embodiments, the autoimmune disease is selected from Coeliac disease, diabetes mellitus type 1 (IDDM), systemic lupus erythematosus (SLE), Sjogren’s syndrome, multiple sclerosis (MS), Hashimoto’s thyroiditis, Graves’ disease, idiopathic thrombocytopenic purpura, and rheumatoid arthritis (RA). In some embodiments, the immune cell composition (such as a pharmaceutical composition) expressing a chimeric receptor polypeptide described herein is administered intravenously, intratumorally, or subcutaneously.

For example, in some embodiments, there is provided a method of treating a target antigen-associated disease (such as cancer, autoimmune disease, or viral infection) in an individual (e.g., human) in need thereof comprising administering to the individual an effective amount of a composition comprising immune cells (such as T cells) presenting on their surface a chimeric receptor polypeptide according to any one of the chimeric receptor polypeptides described herein.

Also contemplated are methods of treating a target antigen-associated disease (such as cancer, autoimmune disease, or viral infection) in an individual in need thereof comprising administering to the individual (e.g., human) a composition comprising a plurality of immune cells (e.g., T cell) expressing different chimeric receptor polypeptides described herein. Thus, in some embodiments, according to any of the methods for treating a target antigen-associated disease in an individual described herein, the composition is a heterogeneous immune cell composition as described herein.

For example, in some embodiments, there is provided a method of treating a target antigen-associated disease (such as cancer, viral infection, or autoimmune disease) in an individual (e.g., human) in need thereof comprising administering to the individual an effective amount of a heterogeneous immune cell composition comprising a plurality of immune cell populations according to any of the embodiments described herein, wherein all of the immune cells in the composition are of the same cell type, wherein each population of immune cells expresses a different chimeric receptor polypeptide than the others, and wherein at least one population of immune cells expresses a chimeric receptor polypeptide that specifically binds to the target antigen. In some embodiments, the immune cells are T cells. In some embodiments, the immune cells are selected from the group consisting of cytotoxic T cells, helper T cells, natural killer T cells, and suppressor T cells. In some embodiments, each population of immune cells expresses a chimeric receptor polypeptide that specifically binds to the same target antigen. In some embodiments, at least one population of immune cells expresses a chimeric receptor polypeptide that specifically binds to a different target antigen (or epitope). In some embodiments, where at least one population of immune cells expresses chimeric receptor polypeptide that specifically binds to a different target antigen, each of the different target antigens (or epitopes) is associated with the target antigen-associated disease.

In some embodiments, there is provided a method of treating a target antigen-associated disease (such as cancer, autoimmune disease, or viral infection) in an individual (e.g., human) in need thereof comprising administering to the individual an effective amount of a heterogeneous immune cell composition comprising a plurality of immune cell populations according to any of the embodiments described herein, wherein at least one population of immune cells is of a different cell type than the others, and wherein at least one population of immune cells expresses a chimeric receptor polypeptide that specifically binds to the target antigen. In some embodiments, all of the populations of immune cells are of different cell types. In some embodiments, the immune cells are T cells. In some embodiments, each population of immune cells is of a cell type selected from the group consisting of cytotoxic T cells, helper T cells, natural killer T cells, and suppressor T cells. In some embodiments, each population of immune cells expresses the same chimeric receptor polypeptide. In some embodiments, at least one population of immune cells expresses a different chimeric receptor polypeptide than the others. In some embodiments, each population of immune cells expresses a different chimeric receptor polypeptide than the others. In some embodiments, each population of immune cells expresses a chimeric receptor polypeptide that specifically binds to the target antigen. In some embodiments, at least one population of the immune cells expresses a chimeric receptor polypeptide that specifically binds to a different target antigen. In some embodiments, where at least one population of immune cells expresses chimeric receptor polypeptide that specifically binds to a different target antigen, each of the different target antigens is associated with the target antigen-associated disease.

In some embodiments, there is provided a method of treating a disease associated with a plurality of target antigens (e.g., cancer, autoimmune disease, viral infection) in an individual (e.g., human) in need thereof comprising administering to the individual an effective amount of a heterogeneous immune cell composition comprising a plurality of immune cell populations according to any of the embodiments described herein, wherein all of the immune cells in the composition are of the same cell type (e.g., all of the immune cells are cytotoxic T cells), wherein each population of the immune cells expresses a different chimeric receptor polypeptide than the others, and wherein for each target antigen of the plurality of target antigens, at least one population of immune cells expresses a chimeric receptor polypeptide that specifically binds to the target antigen. In some embodiments, the immune cells are T cells. In some embodiments, the immune cells are selected from the group consisting of cytotoxic T cells, helper T cells, natural killer T cells, and suppressor T cells.

In some embodiments, there is provided a method of treating a disease associate with a plurality of target antigens (e.g., cancer, autoimmune disease, viral infection) in an individual (e.g., human) in need thereof comprising administering to the individual an effective amount of an immune cell composition comprising a plurality of immune cells according to any of the embodiments described herein, wherein at least one population of the immune cells is of a different cell type than the others, and wherein for each target antigen of the plurality of target antigens, at least one population of immune cells expresses a chimeric receptor polypeptide that specifically binds to the target antigen. In some embodiments, all of the populations of the immune cells are of different cell types. In some embodiments, the immune cells are T cells. In some embodiments, each population of immune cells is of a cell type selected from the group consisting of cytotoxic T cells, helper T cells, natural killer T cells, and suppressor T cells. In some embodiments, each population of immune cells expresses a different chimeric receptor polypeptide than the others.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or environmentally or genetically prone to one or more of the diseases or disorders described herein (such as cancer, autoimmune disease, or viral infection). In some embodiments, the individual has one or more risk factors associated with one or more diseases or disorders described herein.

In some embodiments, there is provided a method of treating a target antigen-associated disease (such as cancer, autoimmune disease, or viral infection) in an individual (e.g., human) in need thereof comprising administering to the individual an effective amount of a composition comprising nucleic acid encoding a chimeric receptor polypeptide according to any of the embodiments described herein.

Cancer treatments can be evaluated, for example, by killing cancer cells, tumor regression, tumor weight or size shrinkage, time to progression, inducing peripheral T cell redistribution (e.g., recruiting T cells to tissues or tumors that express tumor antigen), inhibiting tumor metastasis (e.g., metastasis to lymph nodes), duration of survival, progression free survival, overall response rate, duration of response, alleviating one or more symptoms in an individual having cancer, quality of life, protein expression and/or activity, preventing, inhibiting, or reducing the likelihood of the recurrence of a cancer. Approaches to determining efficacy of the therapy can be employed, including for example, measurement of response through radiological imaging.

In some embodiments, the efficacy of treatment is measured as the percentage tumor growth inhibition (% TGI), calculated using the equation 100-(T/C x 100), where T is the mean relative tumor volume of the treated tumor, and C is the mean relative tumor volume of a non-treated tumor. In some embodiments, the %TGI is about 10%, about 20%, about 30%, about 40%, about 50%, about 60%, about 70%, about 80%, about 90%, about 91%, about 92%, about 93%, about 94% , about 95%, or more than 95%. In some embodiments, the method of reducing tumor size mediated by the immune cells (e.g., T cell,) expressing the chimeric receptor polypeptides described herein (optionally additional CAR/engineered TCR) or pharmaceutical composition thereof can reduce at least about 10% (including for example at least about any of 20%, 30%, 40%, 60%, 70%, 80%, 90%, or 100%) of the tumor size. In some embodiments, the method of inhibiting tumor metastasis (e.g., metastasis to lymph nodes) mediated by the immune cells (e.g., T cell) expressing the chimeric receptor polypeptides described herein (optionally additional CAR/engineered TCR) or pharmaceutical composition thereof can inhibit at least about 10% (including for example at least about any of 20%, 30%, 40%, 60%, 70%, 80%, 90%, or 100%) of the metastasis. In some embodiments, the method of prolonging survival of an individual (such as a human) mediated by the immune cells (e.g., T cell) expressing the chimeric receptor polypeptides described herein (optionally additional CAR/engineered TCR) or pharmaceutical composition thereof can prolongs the survival of the individual by at least any of 1, 2, 3, 4, 5, 6, 7, 8, 9, 10, 11, 12, 18, or 24 months, or more. In some embodiments, the method of prolonging time to cancer progression mediated by the immune cells (e.g., T cell) expressing the chimeric receptor polypeptides described herein (optionally additional CAR/engineered TCR) or pharmaceutical composition thereof can prolong the time to cancer progression by at least any of 1, 2, 3, 4, 5, 6, 7, 8, 9, 10, 11, or 12 weeks, or more. In some embodiments, the immune cells (e.g., T cell) expressing the chimeric receptor polypeptides described herein (optionally additional CAR/engineered TCR) or pharmaceutical composition thereof can increase, enhance, or stimulate an immune response or function in a subject by activating effector cells (e.g., T cells, e.g., CD8+ and/or CD4+ T cells). In some embodiments, the CD4 and/or CD8 T cells in the individual have increased or enhanced priming, activation, proliferation, cytokine release and/or cytolytic activity relative to prior to the administration of the immune cells (e.g., T cell) expressing the chimeric receptor polypeptides described herein (optionally additional CAR/engineered TCR) or pharmaceutical composition thereof.

Viral infection treatments can be evaluated, for example, by viral load, duration of survival, quality of life, protein expression and/or activity.

Autoimmune disease treatment can be evaluated, for example, by autoantibody detection, for example, using immunodiffusion, immunoblotting techniques, immunofluorescence, enzyme immunoassays, or flow cytometry for multiplex bead-based assays.

### Diseases

The engineered immune cells (e.g., T cell) described herein in some embodiments can be useful for treating cancers associated with a target antigen (e.g., BCMA). Cancers that may be treated using any of the methods described herein include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the immune cells of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin’s disease, non-Hodgkin’s lymphoma (indolent and high grade forms), multiple myeloma, plasmacytoma, Waldenstrom’s macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include adrenocortical carcinoma, cholangiocarcinoma, fibrosarcoma, myxosarcoma, liposarcoma, chondrosarcoma, osteosarcoma, and other sarcomas, synovioma, mesothelioma, Ewing’s tumor, leiomyosarcoma, rhabdomyosarcoma, colon carcinoma, stomach cancer, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, thyroid cancer (e.g., medullary thyroid carcinoma and papillary thyroid carcinoma), pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms’ tumor, cervical cancer (e.g., cervical carcinoma and pre-invasive cervical dysplasia), colorectal cancer, cancer of the anus, anal canal, or anorectum, vaginal cancer, cancer of the vulva (e.g., squamous cell carcinoma, intraepithelial carcinoma, adenocarcinoma, and fibrosarcoma), penile cancer, oropharyngeal cancer, esophageal cancer, head cancers (e.g., squamous cell carcinoma), neck cancers (e.g., squamous cell carcinoma), testicular cancer (e.g., seminoma, teratoma, embryonal carcinoma, teratocarcinoma, choriocarcinoma, sarcoma, Leydig cell tumor, fibroma, fibroadenoma, adenomatoid tumors, and lipoma), bladder carcinoma, kidney cancer, melanoma, cancer of the uterus (e.g., endometrial carcinoma), urothelial cancers (e.g., squamous cell carcinoma, transitional cell carcinoma, adenocarcinoma, ureter cancer, and urinary bladder cancer), and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In some embodiments, the cancer to be treated by the immune cells expressing chimeric receptor polypeptides (and optionally CAR/engineered TCR) described herein can be, e.g., acute leukemias (including but not limited to acute myeloid leukemia (AML), B-cell acute lymphoid leukemia (BALL), T-cell acute lymphoid leukemia (TALL), and acute lymphoid leukemia (ALL)), chronic leukemias (including but not limited to chronic myelogenous leukemia (CML) and chronic lymphocytic leukemia (CLL)), multiple myeloma (MM), myelodysplastic syndrome (MDS), myeloproliferative neoplasms (MPNs), chronic myeloid leukemia (CML), and blastic plasmacytoid dendritic cell neoplasm (BPDCN).

The engineered immune cells (e.g., T cell) in other embodiments can be useful for treating infectious diseases by targeting pathogen-associated (such as virally-encoded) antigens. The infection to be prevented or treated, for example, may be caused by a virus, bacteria, protozoa, or parasite. The target antigen may be a pathogenic protein, polypeptide or peptide that is responsible for a disease caused by the pathogen, or is capable of inducing an immunological response in a host infected by the pathogen. Pathogenic antigens which can be targeted by immune cells include, but are not limited to, antigens derived from Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, O111 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Human T cell leukemia virus 1 (HTLV-1), Japanese encephalitis virus, JC virus, Junin virus, Kaposi’s Sarcoma associated herpesvirus (KSHV), Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B 19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In some embodiments, the engineered immune cells (e.g., T cell) expressing chimeric receptor polypeptides (and optionally CAR/engineered TCR) described herein are used for treating oncogenic infectious diseases, such as infection by oncogenic viruses. Oncogenic viruses include, but are not limited to, CMV, EBV, HBV, KSHV, HPV, MCV, HTLV-1, HIV-1, and HCV. The target antigen of the chimeric receptor polypeptide can be a viral oncoprotein including, but not limited to, Tax, E7, E6/E7, E6, HBx, EBNA proteins (e.g., EBNA3 A, EBNA3 C, and EBNA 2), v-cyclin, LANA1, LANA2, LMP-1, k-bZIP, RTA, KSHV K8, and fragments thereof. See Ahuja, Richa, et al., Curr. Sci., 2014.

In some embodiments, the engineered immune cells (e.g., T cell) expressing chimeric receptor polypeptides (and optionally CAR/engineered TCR) described herein are suitable for treating an autoimmune disease. Autoimmune disease, or autoimmunity, is the failure of an organism to recognize its own constituent parts (down to the sub-molecular levels) as “self,” which results in an immune response against its own cells and tissues. Any disease that results from such an aberrant immune response is termed an autoimmune disease. Prominent examples include Coeliac disease, diabetes mellitus type 1 (IDDM), systemic lupus erythematosus (SLE), Sjogren’s syndrome, multiple sclerosis (MS), Hashimoto’s thyroiditis, Graves’ disease, idiopathic thrombocytopenic purpura, and rheumatoid arthritis (RA).

### Articles of Manufacture and Kits

In some embodiments of the invention, there is provided an article of manufacture containing materials useful for the treatment of a target antigen-positive disease such as cancer (for example adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer or thyroid cancer), viral infection (for example infection by CMV, EBV, HBV, KSHV, HPV, MCV, HTLV-1, HIV-1, or HCV), or autoimmune disease (e.g., Coeliac disease, diabetes mellitus type 1 (IDDM), systemic lupus erythematosus (SLE), Sjogren’s syndrome, multiple sclerosis (MS), Hashimoto’s thyroiditis, Graves’ disease, idiopathic thrombocytopenic purpura, and rheumatoid arthritis (RA)). The article of manufacture can comprise a container and a label or package insert on or associated with the container. Suitable containers include, for example, bottles, vials, syringes, etc. The containers may be formed from a variety of materials such as glass or plastic. Generally, the container holds a composition (e.g., engineered immune cell composition expressing the chimeric receptor polypeptide described herein (and optionally CAR/engineered TCR)) which is effective for treating a disea se or disorder described herein, and may have a sterile access port (for example the container may be an intravenous solution bag or a vial having a stopper pierceable by a hypodermic injection needle). At least one active agent in the composition is an immune cell presenting on its surface a chimeric receptor polypeptide of the invention (and optionally CAR/engineered TCR). The label or package insert indicates that the composition is used for treating the particular condition. The label or package insert will further comprise instructions for administering the immune cell composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a target antigen-positive cancer (such as adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer or thyroid cancer). In other embodiments, the package insert indicates that the composition is used for treating a target antigen-positive viral infection (for example infection by CMV, EBV, HBV, KSHV, HPV, MCV, HTLV-1, HIV-1, or HCV). In other embodiments, the package insert indicates that the composition is used for treating a target antigen-positive autoimmune disease (e.g., Coeliac disease, diabetes mellitus type 1 (IDDM), systemic lupus erythematosus (SLE), Sjogren’s syndrome, multiple sclerosis (MS), Hashimoto’s thyroiditis, Graves’ disease, idiopathic thrombocytopenic purpura, and rheumatoid arthritis (RA)). In other embodiments, the package insert indicates that the composition is used for treating a cancer selected from the group consisting of acute leukemias (including but not limited to acute myeloid leukemia (AML), B-cell acute lymphoid leukemia (BALL), T-cell acute lymphoid leukemia (TALL), and acute lymphoid leukemia (ALL)), chronic leukemias (including but not limited to chronic myelogenous leukemia (CML) and chronic lymphocytic leukemia (CLL)), multiple myeloma (MM), myelodysplastic syndrome (MDS), myeloproliferative neoplasms (MPNs), chronic myeloid leukemia (CML), and blastic plasmacytoid dendritic cell neoplasm (BPDCN).

Additionally, the article of manufacture may further comprise a second container comprising a pharmaceutically acceptable buffer, such as bacteriostatic water for injection (BWFI), phosphate-buffered saline, Ringer’s solution and dextrose solution. It may further include other materials desirable from a commercial and user standpoint, including other buffers, diluents, filters, needles, and syringes.

Kits are also provided that are useful for various purposes, e.g., for treatment of a target antigen-positive disease or disorder described herein, optionally in combination with the articles of manufacture. Kits of the invention include one or more containers comprising an immune cell composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition (e.g., pharmaceutical composition) comprising an immune cell (e.g., T cell) presenting on its surface a chimeric receptor polypeptide described herein (and optionally CAR/engineered TCR). In some embodiments, the kit comprises a) a composition (e.g., pharmaceutical composition) comprising an immune cell (e.g., T cell) presenting on its surface a chimeric receptor polypeptide described herein (and optionally CAR/engineered TCR), and b) an effective amount of at least one other agent. In some embodiments, the kit comprises a) a composition (e.g., pharmaceutical composition) comprising an immune cell (e.g., T cell) presenting on its surface a chimeric receptor polypeptide described herein (and optionally CAR/engineered TCR), and b) instructions for administering the immune cell composition to an individual for treatment of a target antigen-positive disease (such as cancer, autoimmune disease, or viral infection). In some embodiments, the kit comprises a) a composition (e.g., pharmaceutical composition) comprising an immune cell (e.g., T cell) presenting on its surface a chimeric receptor polypeptide described herein (and optionally CAR/engineered TCR), b) an effective amount of at least one other agent, and c) instructions for administering the immune cell composition and the other agent(s) to an individual for treatment of a target antigen-positive disease (such as cancer, autoimmune disease, or viral infection). The immune cell composition and the other agent(s) can be present in separate containers or in a single container. For example, the kit may comprise one distinct composition or two or more compositions wherein one composition comprises the immune cell and another composition comprises the other agent.

In some embodiments, the kit comprises a nucleic acid (or set of nucleic acids) encoding a chimeric receptor polypeptide described herein (and optionally CAR/engineered TCR). In some embodiments, the kit comprises a) a nucleic acid (or set of nucleic acids) encoding a chimeric receptor polypeptide described herein (and optionally CAR/engineered TCR), and b) an immune cell (e.g., T cell) for expressing the nucleic acid (or set of nucleic acids). In some embodiments, the kit comprises a) a nucleic acid (or set of nucleic acids) encoding an chimeric receptor polypeptide described herein (and optionally CAR/engineered TCR), and b) instructions for i) expressing the chimeric receptor polypeptide (and optionally CAR/engineered TCR) in an immune cell (e.g., a T cell), ii) preparing a composition comprising the immune cell expressing the chimeric receptor polypeptide (and optionally CAR/engineered TCR), and iii) administering the composition comprising the immune cell expressing the chimeric receptor polypeptide (and optionally CAR/engineered TCR) to an individual for the treatment of a target antigen-positive disease (such as cancer, autoimmune disease, or viral infection). In some embodiments, the immune cell is derived from the individual to be treated. In some embodiments, the kit comprises a) a nucleic acid (or set of nucleic acids) encoding a chimeric receptor polypeptide (and optionally CAR/engineered TCR), b) an immune cell (such as a T cell) for expressing the nucleic acid (or set of nucleic acids), and c) instructions for i) expressing the chimeric receptor polypeptide (and optionally CAR/engineered TCR) in the host cell, ii) preparing a composition comprising the immune cell expressing the chimeric receptor polypeptide (and optionally CAR/engineered TCR), and iii) administering the composition comprising the immune cell (for example T cell) expressing the chimeric receptor polypeptide (and optionally CAR/engineered TCR) to an individual for the treatment of a target antigen-positive disease (such as cancer, autoimmune disease, or viral infection).

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the immune cell compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of an immune cell composition as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the pharmaceutical compositions and instructions for use and packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLARY EMBODIMENTS

Embodiment 1. A chimeric receptor polypeptide comprising:
a) an extracellular target binding domain;
b) an extracellular TCR binding domain;
c) a transmembrane domain comprising a transmembrane domain of a first TCR subunit; and
d) optionally an intracellular domain comprising an intracellular domain of a second TCR subunit,
wherein the first TCR subunit and the second TCR subunit are both selected from the group consisting of CD3ε, CD3γ, CD3δ, TCRα, TCRβ, TCRγ, and TCRδ.

Embodiment 2. The chimeric receptor polypeptide of embodiment 1, wherein the extracellular TCR binding domain comprises a TCR antigen binding domain specifically recognizing a TCR subunit selected from the group consisting of TCRα, TCRβ, TCRγ, TCRδ, CD3ε, CD3δ, CD3γ, and CD3ζ.

Embodiment 3. The chimeric receptor polypeptide of embodiment 2, wherein the TCR antigen binding domain specifically recognizes CD3ε, or TCRγ/δ.

Embodiment 4. The chimeric receptor polypeptide of embodiment 2 or 3, wherein the TCR antigen binding domain is a single chain Fv (scFv) or a single domain antibody (sdAb).

Embodiment 5. The chimeric receptor polypeptide of any one of embodiments 1-4, wherein the extracellular TCR binding domain comprises two or more TCR antigen binding domains arranged in tandem.

Embodiment 6. The chimeric receptor polypeptide of any one of embodiments 1-5, wherein the first TCR subunit and the second TCR subunit are both selected from the group consisting of TCRα, TCRβ, TCRγ, and TCRδ, and wherein the chimeric receptor polypeptide does not comprise a variable region of the extracellular domain of the first TCR subunit or the second TCR subunit.

Embodiment 7. The chimeric receptor polypeptide of embodiment 6, wherein the chimeric receptor polypeptide does not comprise a variable region of the extracellular domain of any of TCRα, TCRβ, TCRγ, and TCRδ.

Embodiment 8. The chimeric receptor polypeptide of any one of embodiments 1-7, wherein the chimeric receptor polypeptide does not comprise an extracellular domain of the first TCR subunit or the second TCR subunit.

Embodiment 9. The chimeric receptor polypeptide of embodiment 8, wherein the chimeric receptor polypeptide does not comprise an extracellular domain of any TCR subunit.

Embodiment 10. The chimeric receptor polypeptide of any one of embodiments 1-9, wherein the chimeric receptor polypeptide does not comprise an intracellular co-stimulatory domain.

Embodiment 11. The chimeric receptor polypeptide of any one of embodiments 1-10, wherein the first TCR subunit and the second TCR subunit are different.

Embodiment 12. The chimeric receptor polypeptide of any one of embodiments 1-11, wherein the first TCR subunit is CD3ε.

Embodiment 13. The chimeric receptor polypeptide of any one of embodiments 1-11, wherein the second TCR subunit is CD3ε.

Embodiment 14. The chimeric receptor polypeptide of any one of embodiments 1-10, wherein the first TCR subunit and the second TCR subunit are the same.

Embodiment 15. The chimeric receptor polypeptide of embodiment 14, wherein the transmembrane domain of the chimeric receptor polypeptide comprises a transmembrane domain of CD3ε, and wherein the intracellular domain of the chimeric receptor polypeptide comprises an intracellular domain of CD3ε.

Embodiment 16. The chimeric receptor polypeptide of embodiment 14, wherein the transmembrane domain of the chimeric receptor polypeptide comprises a transmembrane domain of CD3γ, and wherein the intracellular domain of the chimeric receptor polypeptide comprises an intracellular domain of CD3γ.

Embodiment 17. The chimeric receptor polypeptide of embodiment 14, wherein the transmembrane domain of the chimeric receptor polypeptide comprises a transmembrane domain of CD3δ, and wherein the intracellular domain of the chimeric receptor polypeptide comprises an intracellular domain of CD3δ.

Embodiment 18. The chimeric receptor polypeptide of embodiment 14, wherein the transmembrane domain of the chimeric receptor polypeptide comprises a transmembrane domain of TCRα, and wherein the intracellular domain of the chimeric receptor polypeptide comprises an intracellular domain of TCRα.

Embodiment 19. The chimeric receptor polypeptide of embodiment 14, wherein the transmembrane domain of the chimeric receptor polypeptide comprises a transmembrane domain of TCRβ, and wherein the intracellular domain of the chimeric receptor polypeptide comprises an intracellular domain of TCRβ.

Embodiment 20. The chimeric receptor polypeptide of embodiment 14, wherein the transmembrane domain of the chimeric receptor polypeptide comprises a transmembrane domain of TCRγ, and wherein the intracellular domain of the chimeric receptor polypeptide comprises an intracellular domain of TCRγ.

Embodiment 21. The chimeric receptor polypeptide of embodiment 14, wherein the transmembrane domain of the chimeric receptor polypeptide comprises a transmembrane domain of TCRδ, and wherein the intracellular domain of the chimeric receptor polypeptide comprises an intracellular domain of TCRδ.

Embodiment 22. The chimeric receptor polypeptide of any one of embodiments 1-21, wherein the extracellular target binding domain is N-terminal to the extracellular TCR binding domain.

Embodiment 23. The chimeric receptor polypeptide of any one of embodiments 1-21, wherein the extracellular target binding domain is C-terminal to the extracellular TCR binding domain.

Embodiment 24. The chimeric receptor polypeptide of any one of embodiments 1-23, wherein the extracellular target binding domain comprises a target antigen binding domain specifically recognizing a target antigen.

Embodiment 25. The chimeric receptor polypeptide of embodiment 24, wherein the target antigen binding domain is an scFv, an sdAb, or a designed ankyrin repeat protein (DARPin).

Embodiment 26. The chimeric receptor polypeptide of embodiment 24 or 25, wherein the extracellular target binding domain comprises two or more target antigen binding domains arranged in tandem.

Embodiment 27. The chimeric receptor polypeptide of embodiment 26, wherein the two or more target antigen binding domains each specifically recognizes a same epitope on a same target antigen.

Embodiment 28. The chimeric receptor polypeptide of embodiment 26, wherein the two or more target antigen binding domains each specifically recognizes a different epitope on a same target antigen.

Embodiment 29. The chimeric receptor polypeptide of embodiment 26, wherein the two or more target antigen binding domains each specifically recognizes a different target antigen.

Embodiment 30. The chimeric receptor polypeptide of any one of embodiments 1-29, wherein the target antigen is selected from the group consisting of BCMA, NY-ESO-1, VEGFR2, MAGE-A3, AFP, CD4, CD19, CD20, CD22, CD30, CD33, CD38, CD70, CD123, CEA, EGFR (such as EGFRvIII), GD2, GPC-2, GPC3, HER2, LILRB4, IL-13Rα2, IGF1R, mesothelin, PSMA, ROR1, WT1, NKG2D, CLL1, TGFaRII, TGFbRII, CCR5, CXCR4, CCR4, HPV related antigen, and EBV related antigen (such as LMP1 and LMP2).

Embodiment 31. The chimeric receptor polypeptide of embodiment 30, wherein the target antigen is BCMA.

Embodiment 32. The chimeric receptor polypeptide of any one of embodiments 14-31, wherein the TCR subunit recognized by the TCR antigen binding domain is the same as the first TCR subunit and the second TCR subunit.

Embodiment 33. The chimeric receptor polypeptide of any one of embodiments 1-31, wherein the TCR subunit recognized by the TCR antigen binding domain is different from the first TCR subunit or the second TCR subunit.

Embodiment 34. The chimeric receptor polypeptide of any one of embodiments 1-33, further comprising a first linker connecting the extracellular target binding domain with the extracellular TCR binding domain.

Embodiment 35. The chimeric receptor polypeptide of any one of embodiments 1-34, further comprising a second linker connecting the extracellular target binding domain and/or the extracellular TCR binding domain with the transmembrane domain.

Embodiment 36. The chimeric receptor polypeptide of embodiment 34 or 35, wherein the first linker and/or the second linker is a GS linker, an α-helical linker, a glycine-alanine polymer linker, an alanine-serine polymer linker, or an IgG4-Fc linker.

Embodiment 37. The chimeric receptor polypeptide of any one of embodiments 1-36, further comprising a signal peptide at the N-terminus of the extracellular target binding domain or the extracellular TCR binding domain.

Embodiment 38. The chimeric receptor polypeptide of any one of embodiments 1-37, further comprising a hinge region N-terminal to the transmembrane domain of the chimeric receptor polypeptide.

Embodiment 39. The chimeric receptor polypeptide of embodiment 39, wherein the hinge region comprises the hinge region of CD8.

Embodiment 40. The chimeric receptor polypeptide of any one of embodiments 1-39, comprising from the N-terminus to the C-terminus: a) optional signal peptide - extracellular target binding domain - optional first linker - extracellular TCR binding domain - optional second linker - optional hinge region - transmembrane domain - intracellular domain; or b) optional signal peptide - extracellular TCR binding domain - optional first linker - extracellular target binding domain - optional second linker - optional hinge region - transmembrane domain - intracellular domain.

Embodiment 41. The chimeric receptor polypeptide of any one of embodiments 1 and 34-36, comprising from the N-terminus to the C-terminus: optional signal peptide - anti-BCMA sdAb - first linker - anti-CD3ε scFv - second linker - full length CD3ε without CD3ε signal peptide.

Embodiment 42. The chimeric receptor polypeptide of any one of embodiments 1 and 34-36, comprising from the N-terminus to the C-terminus: optional signal peptide - anti-BCMA sdAb - first linker - anti-CD3ε scFv - second linker - full length CD3γ without CD3γ signal peptide.

Embodiment 43. The chimeric receptor polypeptide of any one of embodiments 1 and 34-36, comprising from the N-terminus to the C-terminus: optional signal peptide - anti-BCMA sdAb - first linker - anti-CD3ε scFv - second linker - full length CD3δ without CD3δ signal peptide.

Embodiment 44. The chimeric receptor polypeptide of any one of embodiments 1 and 34-39, comprising from the N-terminus to the C-terminus: optional signal peptide - anti-BCMA sdAb - first linker - anti-CD3ε scFv - second linker - CD8 hinge region - CD3ε transmembrane domain - CD3ε intracellular domain.

Embodiment 45. The chimeric receptor polypeptide of any one of embodiments 1 and 34-39, comprising from the N-terminus to the C-terminus: optional signal peptide - anti-BCMA sdAb - first linker - anti-CD3ε scFv - second linker - CD8 hinge region - - CD3γ transmembrane domain - CD3γ intracellular domain.

Embodiment 46. The chimeric receptor polypeptide of any one of embodiments 1 and 34-39, comprising from the N-terminus to the C-terminus: optional signal peptide - anti-BCMA sdAb - first linker - anti-CD3ε scFv - second linker - CD8 hinge region - CD3δ transmembrane domain - CD3δ intracellular domain.

Embodiment 47. The chimeric receptor polypeptide of any one of embodiments 1 and 34-36, comprising from the N-terminus to the C-terminus: optional signal peptide - anti-BCMA sdAb - first linker - anti-CD3ε scFv - second linker - TCRα constant region - TCRα transmembrane domain - TCRα intracellular domain.

Embodiment 48. The chimeric receptor polypeptide of any one of embodiments 1 and 34-36, comprising from the N-terminus to the C-terminus: optional signal peptide - anti-BCMA sdAb - first linker - anti-CD3ε scFv - second linker - TCRβ constant region - TCRβ transmembrane domain - TCRβ intracellular domain.

Embodiment 49. The chimeric receptor polypeptide of any one of embodiments 1 and 34-36, comprising from the N-terminus to the C-terminus: optional signal peptide - anti-BCMA sdAb - first linker - anti-CD3ε scFv - second linker - TCRγ constant region - TCRγ transmembrane domain - TCRγ intracellular domain.

Embodiment 50. The chimeric receptor polypeptide of any one of embodiments 1 and 34-36, comprising from the N-terminus to the C-terminus: optional signal peptide - anti-BCMA sdAb - first linker - anti-CD3ε scFv - second linker - TCRδ constant region - TCRδ transmembrane domain - TCRδ intracellular domain.

Embodiment 51. The chimeric receptor polypeptide of any one of embodiments 1 and 34-36, comprising from the N-terminus to the C-terminus: optional signal peptide - anti-BCMA sdAb - first linker - anti-CD3ε scFv - second linker - TCRα transmembrane domain - TCRα intracellular domain.

Embodiment 52. The chimeric receptor polypeptide of any one of embodiments 1 and 34-36, comprising from the N-terminus to the C-terminus: optional signal peptide - anti-BCMA sdAb - first linker - anti-CD3ε scFv - second linker - TCRβ transmembrane domain - TCRβ intracellular domain.

Embodiment 53. The chimeric receptor polypeptide of any one of embodiments 1 and 34-36, comprising from the N-terminus to the C-terminus: optional signal peptide - anti-BCMA sdAb - first linker - anti-CD3ε scFv - second linker - TCRγ transmembrane domain - TCRγ intracellular domain.

Embodiment 54. The chimeric receptor polypeptide of any one of embodiments 1 and 34-36, comprising from the N-terminus to the C-terminus: optional signal peptide - anti-BCMA sdAb - first linker - anti-CD3ε scFv - second linker - TCRδ transmembrane domain - TCRδ intracellular domain.

Embodiment 55. The chimeric receptor polypeptide of any one of embodiments 1 and 34-36, comprising from the N-terminus to the C-terminus: optional signal peptide - anti-BCMA sdAb - first linker - anti-TCR scFv - second linker - TCRα constant region - TCRα transmembrane domain - TCRα intracellular domain.

Embodiment 56. The chimeric receptor polypeptide of any one of embodiments 1 and 34-36, comprising from the N-terminus to the C-terminus: optional signal peptide - anti-BCMA sdAb - first linker - anti- TCR scFv - second linker - TCRβ constant region - TCRβ transmembrane domain - TCRβ intracellular domain.

Embodiment 57. The chimeric receptor polypeptide of any one of embodiments 1 and 34-36, comprising from the N-terminus to the C-terminus: optional signal peptide - anti-BCMA sdAb - first linker - anti-TCR scFv - second linker - TCRγ constant region - TCRγ transmembrane domain - TCRγ intracellular domain.

Embodiment 58. The chimeric receptor polypeptide of any one of embodiments 1 and 34-36, comprising from the N-terminus to the C-terminus: optional signal peptide - anti-BCMA sdAb - first linker - anti-TCR scFv - second linker - TCRδ constant region - TCRδ transmembrane domain - TCRδ intracellular domain.

Embodiment 59. The chimeric receptor polypeptide of any one of embodiments 1 and 34-36, comprising from the N-terminus to the C-terminus: optional signal peptide - first anti-BCMA sdAb - first linker - second anti-BCMA sdAb - second linker - anti-CD3ε scFv - third linker - full length CD3ε without CD3ε signal peptide.

Embodiment 60. The chimeric receptor polypeptide of any one of embodiments 34-59, wherein the first, second, and/or third linker comprises the sequence of any of SEQ ID NOs: 1-21, 67, and 68.

Embodiment 61. An isolated nucleic acid encoding the chimeric receptor polypeptide of any one of embodiments 1-60.

Embodiment 62. A nucleic acid vector comprising one or more nucleic acids of embodiment 61.

Embodiment 63. The nucleic acid vector of embodiment 62, wherein the nucleic acid vector comprises two or more said nucleic acids connected via one or more linking sequences.

Embodiment 64. The nucleic acid vector of embodiment 63, wherein the linking sequence is selected from the group consisting of nucleic acids encoding P2A, T2A, E2A, F2A, BmCPV 2A, and BmIFV 2A, and internal ribosome entry site (IRES) sequence.

Embodiment 65. The nucleic acid vector of any one of embodiments 62-64, comprising a nucleic acid encoding from the N-terminus to the C-terminus: optional first signal peptide -first anti-BCMA sdAb - first linker - first anti-CD3ε scFv - second linker - TCRα constant region - TCRα transmembrane domain - TCRα intracellular domain - P2A - optional second signal peptide - second anti-BCMA sdAb - third linker - second anti-CD3ε scFv - fourth linker - TCRβ constant region - TCRβ transmembrane domain - TCRβ intracellular domain.

Embodiment 66. The nucleic acid vector of any one of embodiments 62-64, comprising a nucleic acid encoding from the N-terminus to the C-terminus: optional first signal peptide -first anti-BCMA sdAb - first linker - first anti-CD3ε scFv - second linker - TCRγ constant region - TCRγ transmembrane domain - TCRγ intracellular domain - P2A - optional second signal peptide - second anti-BCMA sdAb - third linker - second anti-CD3ε scFv - fourth linker - TCRδ constant region - TCRδ transmembrane domain - TCRδ intracellular domain.

Embodiment 67. The nucleic acid vector of any one of embodiments 62-64, comprising a nucleic acid encoding from the N-terminus to the C-terminus: optional first signal peptide -first anti-BCMA sdAb - first linker - first anti-CD3ε scFv - second linker - TCRα transmembrane domain - TCRα intracellular domain - P2A - optional second signal peptide -second anti-BCMA sdAb - third linker - second anti-CD3ε scFv - fourth linker - TCRβ transmembrane domain - TCRβ intracellular domain.

Embodiment 68. The nucleic acid vector of any one of embodiments 62-64, comprising a nucleic acid encoding from the N-terminus to the C-terminus: optional first signal peptide -first anti-BCMA sdAb - first linker - first anti-CD3ε scFv - second linker - TCRγ transmembrane domain - TCRγ intracellular domain - P2A - optional second signal peptide -second anti-BCMA sdAb - third linker - second anti-CD3ε scFv - fourth linker - TCRδ transmembrane domain - TCRδ intracellular domain.

Embodiment 69. The nucleic acid vector of any one of embodiments 62-64, comprising a nucleic acid encoding from the N-terminus to the C-terminus: optional first signal peptide -first anti-BCMA sdAb - first linker - first anti-TCR scFv - second linker - TCRγ constant region - TCRγ transmembrane domain - TCRγ intracellular domain - P2A - optional second signal peptide - second anti-BCMA sdAb - third linker - second anti-TCR scFv - fourth linker -TCRδ constant region - TCRδ transmembrane domain - TCRδ intracellular domain.

Embodiment 70. An isolated immune cell comprising one or more chimeric receptor polypeptides of any one of embodiments 1-60.

Embodiment 71. The isolated immune cell of embodiment 70, wherein the immune cell comprises two or more chimeric receptor polypeptides.

Embodiment 72. An isolated immune cell comprising the nucleic acid of embodiment 61 or the nucleic acid vector of any one of embodiments 62-69.

Embodiment 73. The isolated immune cell of any one of embodiments 70-72, wherein the isolated immune cell is selected from the group consisting of Tαβ cells, Tγδ cells, effector T cells, memory T cells, cytotoxic T cells, T helper cells, Natural Killer T (NKT) cells, regulatory T cells (Tregs), tumor infiltrating lymphocytes (TILs).

Embodiment 74. The isolated immune cell of embodiment 73, which is a T cell.

Embodiment 75. The isolated immune cell of any one of embodiments 70-74, further comprising a chimeric antigen receptor (CAR).

Embodiment 76. The isolated immune cell of any one of embodiments 70-75, further comprising an engineered TCR.

Embodiment 77. A pharmaceutical composition comprising the isolated immune cell of any one of embodiments 70-76, and an optional pharmaceutically acceptable excipient.

Embodiment 78. A method of treating a disease in an individual, comprising administering to the individual an effective amount of the immune cell of any one of embodiments 70-76, or the pharmaceutical composition of embodiment 77.

Embodiment 79. The method of embodiment 78, wherein the pharmaceutical composition is administered intravenously, intratumorally, or subcutaneously.

Embodiment 80. The method of embodiment 78 or 79, wherein the disease is cancer.

Embodiment 81. The method of embodiment 80, wherein the cancer is selected from the group consisting of acute leukemias, chronic leukemias, multiple myeloma (MM), myelodysplastic syndrome (MDS), myeloproliferative neoplasms (MPNs), chronic myeloid leukemia (CML), and blastic plasmacytoid dendritic cell neoplasm (BPDCN).

Embodiment 82. The method of embodiment 81, wherein the acute leukemia is selected from the group consisting of acute myeloid leukemia (AML), B-cell acute lymphoid leukemia (BALL), T-cell acute lymphoid leukemia (TALL), and acute lymphoid leukemia (ALL).

Embodiment 83. The method of embodiment 81, wherein the chronic leukemia is chronic myelogenous leukemia (CML) or chronic lymphocytic leukemia (CLL).

### EXAMPLES

### Example 1. Preparation and Characterization of Immune Cells Expressing Chimeric Receptor Polypeptides

### Construction of Chimeric Receptor Polypeptide Constructs

Chimeric receptor polypeptides comprising from N′ to C′: a signal peptide, a single domain antibody (sdAb) recognizing BCMA (SEQ ID NO: 26), a first GS linker (SEQ ID NO: 3), an scFv recognizing CD3 (UCHT-1) (SEQ ID NO: 24), a second GS linker (SEQ ID NO: 3), and the full chain of a TCR subunit (including the extracellular, transmembrane, and intracellular domains) selected from CD3ε (SEQ ID NO: 28), CD3γ (SEQ ID NO: 29), and CD3δ (SEQ ID NO: 30) were designed and constructed (hereinafter referred to as “sdAbBCMA-anti-CD3 scFv-e” (SEQ ID NO: 43), “sdAbBCMA-anti-CD3 scFv-g” (SEQ ID NO: 44), and “sdAbBCMA-anti-CD3 scFv-d” (SEQ ID NO: 45), respectively). See FIG. 16A as an example. A chimeric receptor polypeptide with tandem sdAb recognizing BCMA (SEQ ID NO: 26) was also constructed, hereinafter referred to as “tandem sdAbBCMA-anti-CD3 scFv-e” (SEQ ID NO: 61), comprising from N′ to C′: a signal peptide, a first sdAb recognizing BCMA (SEQ ID NO: 26), a first GS linker (SEQ ID NO: 1), a second sdAb recognizing BCMA (SEQ ID NO: 26), a second GS linker (SEQ ID NO: 3), an scFv recognizing CD3 (UCHT-1) (SEQ ID NO: 24), a third GS linker (SEQ ID NO: 3), and the full chain of CD3ε (SEQ ID NO: 28). A second set of chimeric receptor polypeptides was constructed to remove the extracellular domain of the TCR subunit CD3ε, CD3γ, or CD3δ, comprising from N′ to C′: a signal peptide, a single domain antibody (sdAb) recognizing BCMA (SEQ ID NO: 26), a first GS linker (SEQ ID NO: 3), an scFv recognizing CD3 (UCHT-1) (SEQ ID NO: 24), a second GS linker (SEQ ID NO: 1), a CD8 hinge region (SEQ ID NO: 31), and transmembrane domain and intracellular domain of a TCR subunit selected from CD3ε (namely, “se” (SEQ ID NO: 40)), CD3γ (namely, “sg” (SEQ ID NO: 41)), and CD3δ (namely, “sd” (SEQ ID NO: 42)); hereinafter referred to as “sdAbBCMA-anti-CD3 scFv-se” (SEQ ID NO: 46), “sdAbBCMA-anti-CD3 scFv-sg” (SEQ ID NO: 47), and “sdAbBCMA-anti-CD3 scFv-sd” (SEQ ID NO: 48), respectively). See FIG. 16B as an example.

TCRα/β/γ/δ subunits lacking corresponding variable regions were constructed, from N′ to C′: constant region of corresponding TCR subunit - transmembrane domain of corresponding TCR subunit - intracellular domain of corresponding TCR subunit, hereinafter referred to as “taC” (SEQ ID NO: 32), “tbC” (SEQ ID NO: 33), “tgC” (SEQ ID NO: 36), and “tdC” (SEQ ID NO: 37), respectively. Nucleic acid constructs encoding chimeric receptor polypeptides comprising an sdAb recognizing BCMA (SEQ ID NO: 26), an scFv recognizing CD3 (UCHT-1) (SEQ ID NO: 24), and taC (SEQ ID NO: 32) and tbC (SEQ ID NO: 33), or tgC (SEQ ID NO: 36) and tdC (SEQ ID NO: 37) were designed and constructed (hereinafter referred to as “sdAbBCMA-anti-CD3 scFv-taC/tbC” (SEQ ID NO: 49), and “sdAbBCMA-anti-CD3 scFv-tgC/tdC” (SEQ ID NO: 50), respectively). Briefly, sdAbBCMA-anti-CD3 scFv-taC/tbC (SEQ ID NO: 49) comprises from N′ to C′: [a first signal peptide, a first sdAb recognizing BCMA (SEQ ID NO: 26), a first GS linker (SEQ ID NO: 67), an first scFv recognizing CD3 (UCHT-1) (SEQ ID NO: 24), a second GS linker (SEQ ID NO: 3), taC (SEQ ID NO: 32)] - P2A (SEQ ID NO: 66) - [a second signal peptide, a second sdAb recognizing BCMA (SEQ ID NO: 26), a third GS linker (SEQ ID NO: 67), an second scFv recognizing CD3 (UCHT-1) (SEQ ID NO: 24), a fourth GS linker (SEQ ID NO: 3), tbC (SEQ ID NO: 33)]. sdAbBCMA-anti-CD3 scFv-tgC/tdC (SEQ ID NO: 50) comprises from N′ to C′: [a first signal peptide, a first sdAb recognizing BCMA (SEQ ID NO: 26), a first GS linker (SEQ ID NO: 67), an first scFv recognizing CD3 (UCHT-1) (SEQ ID NO: 24), a second GS linker (SEQ ID NO: 3), tgC (SEQ ID NO: 36)] - P2A (SEQ ID NO: 66) - [a second signal peptide, a second sdAb recognizing BCMA (SEQ ID NO: 26), a third GS linker (SEQ ID NO: 67), an second scFv recognizing CD3 (UCHT-1) (SEQ ID NO: 24), a fourth GS linker (SEQ ID NO: 3), tdC (SEQ ID NO: 37)].

TCRα/β/γ/δ subunits lacking corresponding extracellular domains were constructed, from N′ to C′: transmembrane domain of corresponding TCR subunit - intracellular domain of corresponding TCR subunit, hereinafter referred to as “sta” (SEQ ID NO: 34), “stb” (SEQ ID NO: 35), “stg” (SEQ ID NO: 38), and “std” (SEQ ID NO: 39), respectively. Nucleic acid constructs encoding a second set of chimeric receptor polypeptides having similar configurations as discussed above but lacking the extracellular domains of the TCRαβ or TCRγδ subunits were also made (hereinafter referred to as “sdAbBCMA-anti-CD3 scFv-sta/stb” (SEQ ID NO: 59) and “sdAbBCMA-anti-CD3 scFv-stg/std” (SEQ ID NO: 60), respectively). Briefly, sdAbBCMA-anti-CD3 scFv-sta/stb (SEQ ID NO: 59) comprises from N′ to C′: [a first signal peptide, a first sdAb recognizing BCMA (SEQ ID NO: 26), a first GS linker (SEQ ID NO: 67), an first scFv recognizing CD3 (UCHT-1) (SEQ ID NO: 24), a second GS linker (SEQ ID NO: 3), sta (SEQ ID NO: 34)] - P2A (SEQ ID NO: 66) - [a second signal peptide, a second sdAb recognizing BCMA (SEQ ID NO: 26), a third GS linker (SEQ ID NO: 67), an second scFv recognizing CD3 (UCHT-1) (SEQ ID NO: 24), a fourth GS linker (SEQ ID NO: 3), stb (SEQ ID NO: 35)]. sdAbBCMA-anti-CD3 scFv-stg/std (SEQ ID NO: 60) comprises from N′ to C′: [a first signal peptide, a first sdAb recognizing BCMA (SEQ ID NO: 26), a first GS linker (SEQ ID NO: 67), an first scFv recognizing CD3 (UCHT-1) (SEQ ID NO: 24), a second GS linker (SEQ ID NO: 3), stg (SEQ ID NO: 38)] - P2A (SEQ ID NO: 66) - [a second signal peptide, a second sdAb recognizing BCMA (SEQ ID NO: 26), a third GS linker (SEQ ID NO: 67), an second scFv recognizing CD3 (UCHT-1) (SEQ ID NO: 24), a fourth GS linker (SEQ ID NO: 3), std (SEQ ID NO: 39)].See FIG. 16D as an example.

Nucleic acid construct encoding chimeric receptor polypeptides comprising a sdAb recognizing BCMA (SEQ ID NO: 26), a TCR scFv recognizing TCR constant domain (B1.1 monoclonal anti-TCRγ/δ Ab; SEQ ID NO: 27), and tgC (SEQ ID NO: 36) and tdC (SEQ ID NO: 37) was designed and constructed, hereinafter referred to as “sdAbBCMA-anti-TCR Ab-tgC/tdC” (SEQ ID NO: 64). Briefly, sdAbBCMA-anti-TCR Ab-tgC/tdC (SEQ ID NO: 64) comprises from N′ to C′: [a first signal peptide, a first sdAb recognizing BCMA (SEQ ID NO: 26), a first GS linker (SEQ ID NO: 4), a first TCR antibody recognizing TCR constant domain (SEQ ID NO: 27), a second GS linker (SEQ ID NO: 3), tgC (SEQ ID NO: 36)] - P2A (SEQ ID NO: 66) - [a second signal peptide, a second sdAb recognizing BCMA (SEQ ID NO: 26), a third GS linker (SEQ ID NO: 4), a second TCR antibody recognizing TCR constant domain (SEQ ID NO: 27), a fourth GS linker (SEQ ID NO: 3), tdC (SEQ ID NO: 37)]. See FIG. 16C as an example.

Nucleic acids encoding the above chimeric receptor polypeptides were cloned into lentiviral vector under the control of an EF1α promoter (pLVX-EF1A) generated in house. Briefly, the constitutively active human cytomegalovirus immediate early promoter (P_{CMV IE}) located just upstream of the multiple cloning site (MCS) and the puromycin resistance gene within the H1V-1-based lentiviral vector pLVX-Puro (Clontech #632164) was replaced with human elongation factor 1α promoter (hEF1α) by EcoRI and BamHI, hereinafter referred to as pLVX-EF1A lentiviral vector.

### Packaging of the Lentiviral Vector

A lentivirus packaging plasmid mixture including pMDLg/pRRE (Addgene#12251), pRSV-Rev (Addgene#12253), and pMD2.G (Addgene#12259) was pre-mixed with an pLVX-EF1A lentiviral vector carrying the corresponding nucleic acid encoding the above chimeric receptor polypeptide at a pre-optimized ratio with polyetherimide (PEI), then mixed properly and incubated at room temperature for 5 minutes. The transfection mix was then added dropwise to 293-T cells and mixed gently. The cells were then incubated overnight in a 37° C. and 5% CO₂ cell incubator. The next day, supernatant was collected after centrifugation at 4° C., 500 g for 10 min.

The supernatant was filtered through a 0.45 µm PES filter, then concentrated with ultracentrifugation. After ultracentrifugation, the supernatant was carefully discarded and virus pellets were rinsed cautiously with pre-chilled DPBS. The concentration of virus was then measured. Virus was aliquoted properly, then stored at -80° C. immediately. The virus titer was determined by functional transduction on T cell line.

### T Cell Preparation

Leukocytes were collected from R10 medium, then mixed with 0.9% NaCl solution at 1:1 (v/v) ratio. 3 mL lymphoprep medium was added to a 15 mL centrifuge tube, then 6 mL of diluted lymphocyte mix was slowly added to layer on top of lymphoprep. The lymphocyte mix was centrifuged at 800 g for 30 minutes without brakes at 20° C. Lymphocyte buffy coat was then collected with a 200 µL pipette. The harvested fraction was diluted with at least 6 folds of 0.9% NaCl or R10 to reduce the density of the solution. The harvested fraction was then centrifuged at 250 g for 10 minutes at 20° C. The supernatant was aspirated completely, and 10 mL of R10 was added to the cell pellet. The mixture was further centrifuged at 250 g for 10 minutes at 20° C. The supernatant was then aspirated. 2 mL 37° C. pre-warmed R10 with 100 IU/mL IL-2 was added to the cell pellet, and the cell pellet was re-suspended softly. The number of cells was then counted, and the PBMC sample was ready for later experiments.

Human T cells were purified from PBMCs using Miltenyi Pan T cell isolation kit (Cat#130-096-535), following the manual protocol. The prepared T cells were subsequently pre-activated for 48 hours with human T cell Activation/Expansion kit (Milteny#130-091-441). An optimal activation of T cells is accomplished by using one loaded Anti-Biotin MACSiBead Particle per two cells (bead-to-cell ratio 1:2).

### T Cell Transfection

The pre-activated T cells were collected and suspended/re-suspended in a 1640 medium containing the final concentration of 300 IU/mL IL-2. The lentivirus was diluted to MOI = 5 with the same medium. 1E+06 pre-activated T cells were transduced with the diluted lentivirus with the presence of 8 µg/ml polybrene with centrifugation at 1000 g, 32° C. for 1 h. The transduced cells were then transferred to the cell culture incubator for transgene expression under suitable conditions. The next day, the transduced cells were centrifuged and replaced with fresh 1640 media (supplement with 300 IU/mL IL-2), cell concentration was measured every 2 days, and fresh 1640 media (supplement with 300 IU/mL IL-2) was added to continue T cell expansion. T cells expressing the chimeric receptor polypeptides are referred to as “STS-T cells.”

### Cell Cytotoxicity Assays - Lactate Dehydrogenase (LDH) Assay

Cytotoxicity of the STS-T cells were determined in a 6-24 hour co-culture assay. In the experiments, STS-T cells or un-transfected T cells (UnT) were collected by centrifugation, then diluted to the desired concentrations by 1640 phenol red free medium (Invitrogen) with 1.25% heat inactivated FBS (Invitrogen). Target cells with strong expression of target antigen (i.e., BCMA) were used, such as CHO-K1/BCMA cells (in house engineered CHO cell line constitutively expressing full-length human BCMA protein) or RPMI-8226 cells (multiple myeloma cell line, ATCC, Cat.#CRM-CCL-155, Lot#63990046).NCI-H929 cells (myeloma cell line, ATCC, Cat.# CRL-9068, Lot# 61685273). STS-T cells and target cells were co-cultured at different effector to target ratios (E:T) at 37° C. for 6-24 hours in 96 well plate. Additional wells contained assay buffer only (1640 phenol red free medium plus 1.25% hiFBS), target cell only (T), effector cell only (E), and max release of target cell (target cells with 1% solution of triton-X 100). Each condition was performed in triplicate, and the cytotoxicity of STS-T cells on target cells was detected by LDH assay kit (Roche, Cat. # 11644793001). After co-culture for 6-24 hours, the assay plate was centrifuged, and the supernatant was transferred to a new 96-well plate. Equal volume of the LDH assay reagent was added into each well containing supernatant according to manufacture’s manual. The reaction was incubated for about 30 min at 15° C.~25° C. Then absorbance at 492 nm and 650 nm was measured using FlexStation® reader (Molecular Devices). Tumor cell lysis percentage was calculated using the formula:

%Target cell lysis = (OD_{E:T} - OD_{T} - OD_{E} + OD_{Assay}_{buffer})/(OD_{Max}ᵣₑₗₑₐₛₑ - OD_{T})^{∗} 100.

### Cell Cytotoxicity Assay - Luciferase Assay

Cytotoxicity of the STS-T cells was determined in a 6-24 hour co-culture assay. In the experiments, STS-T cells or un-transfected T cells (UnT) were collected by centrifugation, then diluted to the desired concentrations by 1640 phenol red free medium (Invitrogen) with 1.25% heat inactivated FBS (Invitrogen). Tumor cells with strong expression of BCMA and luciferase were used, such as NCI-H929-Luc cells (myeloma cell line, ATCC, Cat.# CRL-9068, Lot# 61685273). STS-T cells and target cells were co-cultured at different effector to target ratios (E:T) at 37° C. for 6-24 hours in a 96 well plate. Additional wells were loaded with target cell only (T) and max release of target cell (target cells with 1% solution of triton-X 100). Each condition was performed in triplicate, and the cytotoxicity of STS-T cells on target cells were detected by One-Glo assay kit (Promega, Cat. # E6110).

After co-culture, the assay plate was briefly centrifuged (the supernatant was removed), then equal volume (equal to medium) of One-Glo assay reagent was added into each well containing cells according to manufacture’s manual. The plate was incubated for about 3 min at room temperature. Then luciferase signal was measured using PHERAstar® plus reader (BMG labtech). The percentage of tumor cell lysis was calculated using the below formula:

%Target cell lysis = (1 - (RLU_{E:T} - RLU_{Max}ᵣₑₗₑₐₛₑ)/(RLU_{T} - RLU_{Max release}))^{∗} 100.

### Cytokine Release Detection

The transferred out supernatant from assay plate in cytotoxicity assays was collected for cytokine release analysis. The supernatant was transferred to a new 96-well plate, then HTRF® reagents (human IFN gamma kit, Cisbio, Cat#62HIFNGPEH) were added into each well for the detection of INFγ release. IFNγ Standard (from the IFN gamma kit) was used to determine the amount of IFNγ. The antibodies labeled with the HTRF donor and acceptor were pre-mixed and added in a single dispensing step.

The 4 Parameter Logistic (4PL) curve is commonly recommended for fitting an ELISA standard curve. This regression enables the accurate measurement of an unknown sample across a wider range of concentrations than linear analysis, making it ideally suitable for the analysis of biological systems like cytokine releases.

### Example 2. Cell Cytotoxicity and Cytokine Release Assays of STS-T Cells

STS-T cells described in Example 1 (or un-transfected T cells (UnT), control) and target cells were collected by centrifugation, then diluted to the desired concentrations with assay buffer (1640 phenol red free medium plus 1.25% hiFBS). STS-T cells (or control UnT) and RPMI-8226 cells (multiple myeloma cell line) were then co-cultured at E:T of 1.25 at 37° C. for 20 hours in a 96 well plate. Supernatant after co-culture was transferred out from each well for detection of LDH release and IFNγ secretion. Detailed methods are described in Example 1.

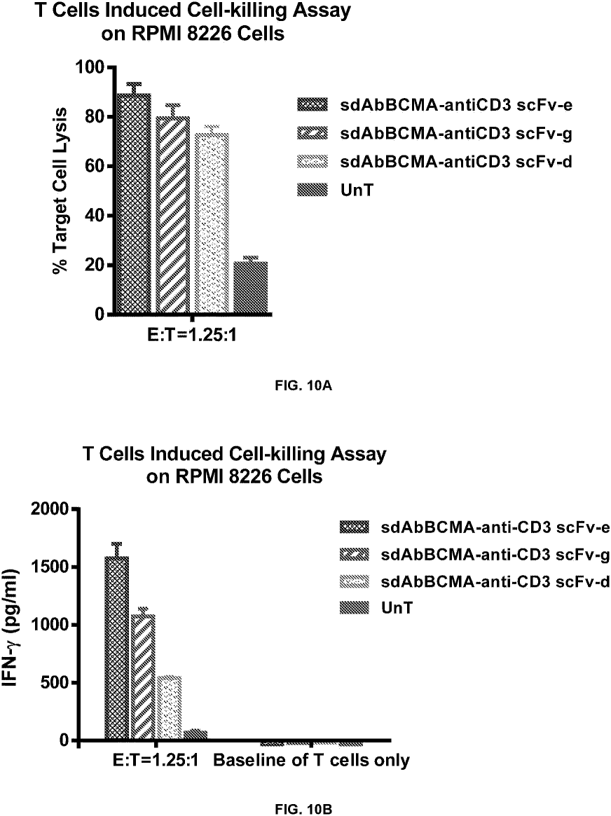

FIG. 10A and FIG. 10B provide cell killing assay results of various STS-T cells on RPMI-8226 cells (multiple myeloma cell line, ATCC, Cat.#CRM-CCL-155, Lot#63990046), including STS-T cells expressing sdAbBCMA-anti-CD3 scFv-e, sdAbBCMA-anti-CD3 scFv-g, and sdAb BCMA-anti CD3 scFv-d. As shown in FIG. 10A and FIG. 10B, sdAbBCMA-anti-CD3 scFv-e, sdAbBCMA-anti-CD3 scFv-g, and sdAbBCMA-anti-CD3 scFv-d all showed significant cytotoxicity on RPMI-8226 cells (multiple myeloma cell line, ATCC, Cat.#CRM-CCL-155, Lot#63990046) and strong induction on IFNγ release, as compared to untransfected T cells (UnT), among which sdAbBCMA-anti-CD3 scFv-e showed the strongest cytotoxicity (FIG. 10A) and IFNγ release (FIG. 10B).

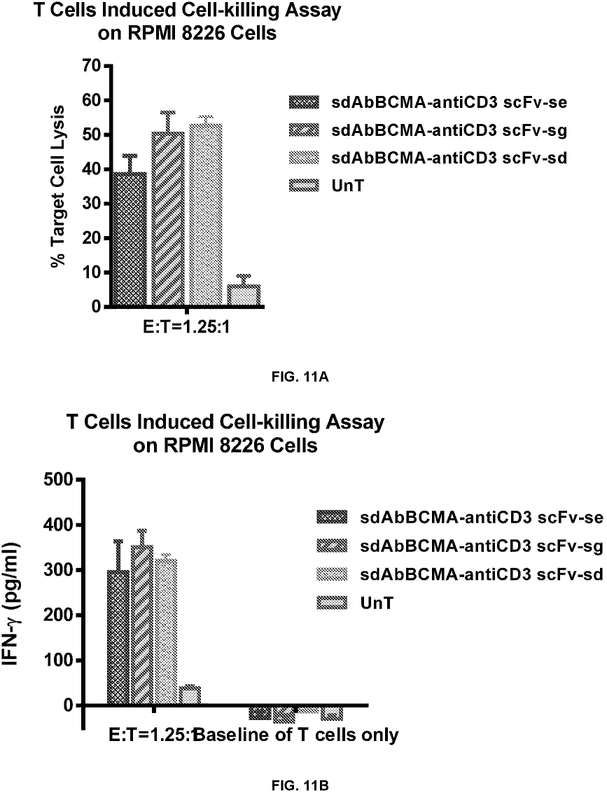

FIG. 11A and FIG. 11B provide cell killing assay results of various STS-T cells on RPMI-8226 cells, including STS-T cells expressing sdAbBCMA-anti-CD3 scFv-se, sdAbBCMA-anti-CD3 scFv-sg, and sdAbBCMA-anti-CD3 scFv-sd. As shown in FIG. 11A and FIG. 11B, sdAbBCMA-anti-CD3 scFv-se, sdAbBCMA-anti-CD3 scFv-sg, and sdAbBCMA-anti-CD3 scFv-sd all showed significant cytotoxicity on RPMI-8226 cells and strong induction on IFNγ release, as compared to untransfected T cells (UnT).

These results demonstrate that chimeric receptor polypeptides with or without the extracellular domain of CD3ε/CD3γ/CD3δ are both effective in inducing cytotoxicity on target tumor cells and cytokine release.

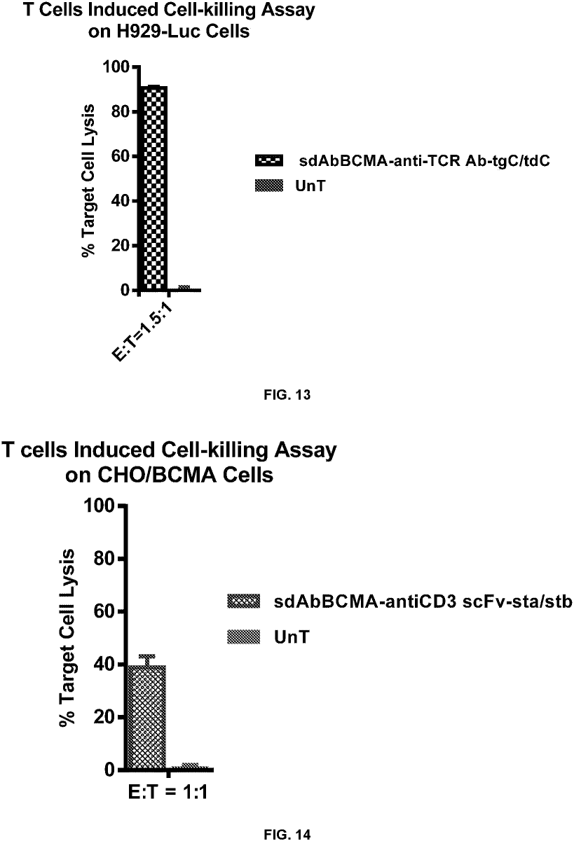

FIG. 13 shows cell killing assay results of STS-T cells expressing sdAbBCMA-anti-TCR Ab-tgC/tdC on NCI-H929-Luc cells (in house made luciferase expressed stable cell line on NCI-H929 cells). As shown in FIG. 13, sdAbBCMA-anti-TCR Ab-tgC/tdC showed significant cytotoxicity on target tumor cells as compared to untransfected T cells.

FIG. 14 shows cell killing assay results of STS-T cells expressing sdAbBCMA-anti-CD3 scFv-sta/stb on CHO-K1/BCMA cells (in house engineered CHO cell line constitutively expressing full length human BCMA protein). As shown in FIG. 14, sdAbBCMA-anti-CD3 scFv-sta/stb showed significant cytotoxicity on target tumor cells as compared to untransfected T cells.

These results demonstrate that chimeric receptor polypeptides with or without the constant domain of TCRα/β/γ/δ are both effective in inducing cytotoxicity on target tumor cells.

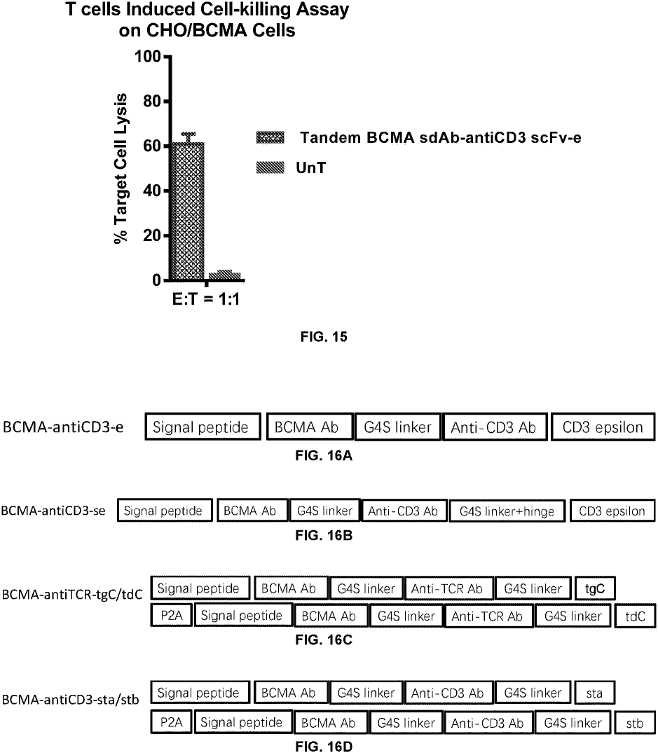

FIG. 15 shows cell killing assay results of STS-T cells expressing tandem sdAbBCMA-anti-CD3 scFv-e on CHO-K1/BCMA cells (in house engineered CHO cell line constitutively expressing full length human BCMA protein). As shown in FIG. 15, tandem sdAbBCMA-anti-CD3 scFv-e showed significant cytotoxicity on target tumor cells as compared to untransfected T cells.

### Example 3. TCR and CD3 Expression in STS-T Cells

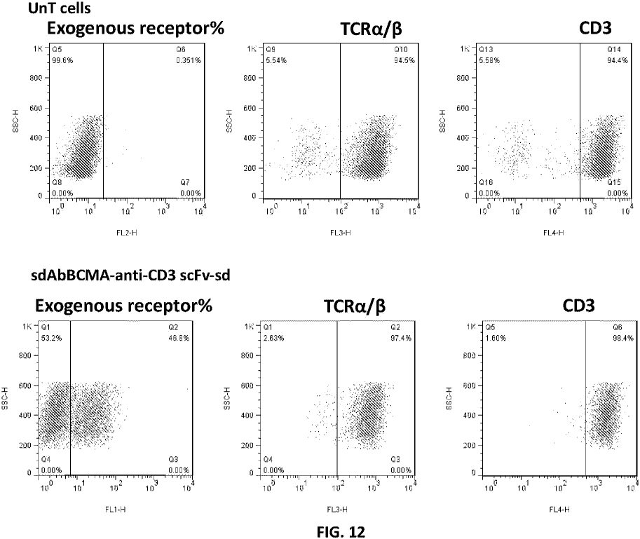

sdAbBCMA-anti-CD3 scFv-sd (SEQ ID NO: 48) from Example 1 was constructed and expressed in T cells. On Day 5 after transduction, T cells were collected and stained with BCMA protein (human BCMA Protein Fc Tag, ACRO, Cat#BC7-H5254) for detection of exogenous receptor expression, anti-TCRα/β (Biolegend, Cat#306710) for endogenous TCR expression, and anti-CD3 antibody (Biolegend, Cat#300439) for endogenous CD3 expression. As shown in FIG. 12, compared to untransfected T cells (UnT), sdAbBCMA-anti-CD3 scFv-sd maintained the expression level of endogenous TCR α/β and endogenous CD3 as untransfected T cells. This demonstrates that chimeric receptor polypeptides of the present invention did not affecting endogenous TCR expression and/or function.

### SEQUENCE LISTING

SEQ ID NO: 1 (linker)

GGGGS

SEQ ID NO: 2 (linker)

GGGGSGGGGS

SEQ ID NO: 3 (linker)

GGGGSGGGGSGGGGS

SEQ ID NO: 4 (linker)

GGGGSGGGGSGGGGSGGGGS

SEQ ID NO: 5 (linker)

GGSGGS

SEQ ID NO: 6 (linker)

SGGGGS

SEQ ID NO: 7 (linker)

GRAGGGGAGGGG

SEQ ID NO: 8 (linker)

GRAGGG

SEQ ID NO: 9 (linker; n is an integer of at least 1)

(G)n

SEQ ID NO: 10 (linker; n is an integer of at least 1)

(GS)n

SEQ ID NO: 11 (linker; n is an integer of at least 1)

(GSGGS)n

SEQ ID NO: 12 (linker; n is an integer of at least 1)

(GGGS)n

SEQ ID NO: 13 (linker; n is an integer of at least 1)

(GGGGS)n

SEQ ID NO: 14 (linker)

GG

SEQ ID NO: 15 (linker)

GGSG

SEQ ID NO: 16 (linker)

GGSGG

SEQ ID NO: 17 (linker)

GSGSG

SEQ ID NO: 18 (linker)

GSGGG

SEQ ID NO: 19 (linker)

GGGSG

SEQ ID NO: 20 (linker)

GSSSG

SEQ ID NO: 21 (IgG4-Fc-linker)

ESKYGPPCPPCPAPEFEGGPSVFLFPPKPKDTLMISRTPEVTCVVVDVSQ
EDPEVQFNWYVDGVEVHNAKTKPREEQFQSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK

SEQ ID NO: 22 (anti-CD3 sdAb)

QVKLEESGGGLVQAGGSLRVSCTASGRTFDTMGWFRQAPGKEREFVAAVR
WSSGNTLYGNTVKGRFTISRDTATNTVYLQMSSLKHEDTAVYYCAARVGG
RGAADHWGQGTQVTVSS

SEQ ID NO: 23 (anti-CD3 sdAb)

QVQLVESGGGLVQAGGSLRLSCAASGRTFSSRAVGWFRQAPGKERQFVAA
IDSGGGETGYADSVKGRFAISRDNAKNTVYLQMNSLKPEDTAVYYCAVAD
LLVTWPRAYKYWGQGTQVTVSS

SEQ ID NO: 24 (anti-CD3 scFv (UCHT1))

MDIQMTQTTSSLSASLGDRVTISCRASQDIRNYLNWYQQKPDGTVKLLIY
YTSRLHSGVPSKFSGSGSGTDYSLTISNLEQEDIATYFCQQGNTLPWTFA
GGTKLEIKGGGGSGGGGSGGGGSGGGGSEVQLQQSGPELVKPGASMKISC
KASGYSFTGYTMNWVKQSHGKNLEWMGLINPYKGVSTYNQKFKDKATLTV
DKSSSTAYMELLSLTSEDSAVYYCARSGYYGDSDWYFDVWGQGTTLTVFS

SEQ ID NO: 25 (MMP sequence)

PLGLAG

SEQ ID NO: 26 (anti-BCMA sdAb)

EVQLVESGGGLVQAGGSLRLSCAASGRTFTMGWFRQAPGKEREFVAAISL
SPTLAYYAESVKGRFTISRDNAKNTVVLQMNSLKPEDTALYYCAADRKSV
MSIRPDYWGQGTQVTVSS

SEQ ID NO: 27 (anti-TCRγ/δ Ab)

QVQLQQSGPELVKPGASVKISCKISDYTFSNSWMNWVKQRPGQGLEWIGR
IYPGDGDTKYNGEFKAKATLTADKSARTAYMQLNNLTSVDSAVYFCARWE
RYDGGFTYWGQGTLVTVSPGGGGSGGGGSGGGGSDIQMTQSPASLSASVG
ETVTITCRASENIYSYLSWYQQKQGKSPQLLVYYANTLAEGVPSRFSGSG
YGTQFSLKINSLQPVDFGNYYCQHHYGLPPTFGGGTKLEIK

SEQ ID NO: 28 (CD3ε; CD3ε extracellular domain is underlined; signal peptide is bolded)

MQSGTHWRVLGLCLLSVGVWGQDGNEEMGGITQTPYKVSISGTTVILTCP QYPGSEILWQHNDKNIGGDEDDKNIGSDEDHLSLKEFSELEQSGYYVCYP RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI

SEQ ID NO: 29 (CD3γ; CD3γ extracellular domain is underlined; signal peptide is bolded)

MEQGKGLAVLILAIILLQGTLAQSIKGNHLVKVYDYQEDGSVLLTCDAEA KNITWFKDGKMIGFLTEDKKKWNLGSNAKDPRGMYQCKGSQNKSKPLQVY YRMCQNCIELNAATISGFLFAEIVSIFVLAVGVYFIAGQDGVRQSRASDK
QTLLPNDQLYQPLKDREDDQYSHLQGNQLRRN

SEQ ID NO: 30 (CD3δ; CD3δ extracellular domain is underlined; signal peptide is bolded)

MEHSTFLSGLVLATLLSQVSPFKIPIEELEDRVFVNCNTSITWVEGTVGT LLSDITRLDLGKRILDPRGIYRCNGTDIYKDKESTVQVHYRMCQSCVELD PATVAGIIVTDVIATLLLALGVFCFAGHETGRLSGAADTQALLRNDQVYQ
PLRDRDDAQYSHLGGNWARNK

SEQ ID NO: 31 (CD8 hinge)

TTTPAPRPPTPAPTIASQPLSLRPEACRPAAGGAVHTRGLDFACD

SEQ ID NO: 32 (taC; transmembrane domain and intracellular domain are bolded)

PNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDVKL
VEKSFETDTNLNFQNLSVIGFRILLLKVAGFNLLMTLRLWSS

SEQ ID NO: 33 (tbC; transmembrane domain and intracellular domain are bolded)

EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILYE
ILLGKATLYAVLVSALVLMAMVKRKDF

SEQ ID NO: 34 (sta; transmembrane domain and intracellular domain of TCRα)

VIGFRILLLKVAGFNLLMTLRLWSS

SEQ ID NO: 35 (stb; transmembrane domain and intracellular domain of TCRα)

ILLGKATLYAVLVSALVLMAMVKRKDF

SEQ ID NO: 36 (tgC; transmembrane domain and intracellular domain are bolded)

DKQLDADVSPKPTIFLPSIAETKLQKAGTYLCLLEKFFPDVIKIHWQEKK
SNTILGSQEGNTMKTNDTYMKFSWLTVPEKSLDKEHRCIVRHENNKNGVD
QEIIFPPIKTDVITMDPKDNCSKDANDTLLLQLTNTSAYYMYLLLLLKSV VYFAIITCCLLRRTAFCCNGEKS

SEQ ID NO: 37 (tdC; transmembrane domain and intracellular domain are bolded)

SQPHTKPSVFVMKNGTNVACLVKEFYPKDIRINLVSSKKITEFDPAIVIS
PSGKYNAVKLGKYEDSNSVTCSVQHDNKTVHSTDFEVKTDSTDHVKPKET
ENTKQPSKSCHKPKAIVHTEKVNMMSLTVLGLRMLFAKTVAVNFLLTAKL FFL

SEQ ID NO: 38 (stg; transmembrane domain and intracellular domain of TCRγ)

YYMYLLLLLKSVVYFAIITCCLLRRTAFCCNGEKS

SEQ ID NO: 39 (std; transmembrane domain and intracellular domain of TCRδ)

LGLRMLFAKTVAVNFLLTAKLFFL

SEQ ID NO: 40 (se; CD3ε without extracellular domain)

VMSVATIVIVDICITGGLLLLVYYWSKNRKAKAKPVTRGAGAGGRQRGQN
KERPPPVPNPDYEPIRKGQRDLYSGLNQRRI

SEQ ID NO: 41 (sg; CD3γ without extracellular domain)

GFLFAEIVSIFVLAVGVYFIAGQDGVRQSRASDKQTLLPNDQLYQPLKDR
EDDQYSHLQGNQLRRN

SEQ ID NO: 42 (sd; CD3δ without extracellular domain)

GIIVTDVIATLLLALGVFCFAGHETGRLSGAADTQALLRNDQVYQPLRDR
DDAQYSHLGGNWARNK

SEQ ID NO: 43 (sdAbBCMA-anti-CD3 scFv-e; BCMA sdAb is underlined; anti-CD3 scFv is bolded; CD3s is italicized; signal peptide sequence is bolded and italicized; linker is squared)

MQSGTHWRVLGLCLLSVGVWGQ EVQLVESGGGLVQAGGSLRLSCAASGRT FTMGWFRQAPGKEREFVAAISLSPTLAYYAESVKGRFTISRDNAKNTVVL QMNSLKPEDTALYYCAADRKSVMSIRPDYWGQGTQVTVSSGGGGSGGGGS
GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSGGGGSGGGGSDGNEEMGGITQTPYKVSISGTTVILTCPQY PGSEILWQHNDKNIGGDEDDKNIGSDEDHLSLKEFSELEQSGYYVCYPRG SKPEDANFYLYLRARVCENCMEMDVMSVATIVIVDICITGGLLLLVYYWS KNRKAKAKPVTRGAGAGGRQRGQNKERPPPVPNPDYEPIRKGQRDLYSGL NQRRI

SEQ ID NO: 44 (sdAbBCMA-anti-CD3 scFv-g; BCMA sdAb is underlined; anti-CD3 scFv is bolded; CD3γ is italicized; signal peptide sequence is bolded and italicized; linker is squared)

MEQGKGLAVLILAIILLQGTLA EVQLVESGGGLVQAGGSLRLSCAASGRT FTMGWFRQAPGKEREFVAAISLSPTLAYYAESVKGRFTISRDNAKNTVVL QMNSLKPEDTALYYCAADRKSVMSIRPDYWGQGTQVTVSSGGGGSGGGGS
GGGGMDIQMTQTTSSLSASLGDRVTISCRASQDIRNYLNWYQQKPDGTVK LLIYYTSRLHSGVPSKFSGSGSGTDYSLTISNLEQEDIATYFCQQGNTLP WTFAGGTKLEIKGGGGSGGGGSGGGGSGGGGSEVQLQQSGPELVKPGASM KISCKASGYSFTGYTMNWVKQSHGKNLEWMGLINPYKGVSTYNQKFKDKA TLTVDKSSSTAYMELLSLTSEDSAVYYCARSGYYGDSDWYFDVWGQGTTL TVFSGGGGSGGGGSGGGGSQSIKGNHLVKVYDYQEDGSVLLTCDAEAKNI TWFKDGKMIGFLTEDKKKWNLGSNAKDPRGMYQCKGSQNKSKPLQVYYRM CQNCIELNAATISGFLFAEIVSIFVLAVGVYFIAGQDGVRQSRASDKQTL LPNDQLYQPLKDREDDQYSHLQGNQLRRN

SEQ ID NO: 45 (sdAbBCMA-anti-CD3 scFv-d; BCMA sdAb is underlined; anti-CD3 scFv is bolded; CD3δ is italicized; signal peptide sequence is bolded and italicized; linker is squared)

MQSGTHWRVLGLCLLSVGVWGQ EVQLVESGGGLQAGGSLRLSCAASGRTF TMGWFRQAPGKEREFVAAISLSPTLAYYAESVKGRFTISRDNAKNTVVLQ MNSLKPEDTALYYCAADRKSVMSIRPDYWGQGTQVTVSSGGGGSGGGGSG
GGGSMDIQMTQTTSSLSASLGDRVTISCRASQDIRNYLNWYQQKPDGTVK LLIYYTSRLHSGVPSKFSGSGSGTDYSLTISNLEQEDIATYFCQQGNTLP WTFAGGTKLEIKGGGGSGGGGSGGGGSGGGGSEVQLQQSGPELVKPGASM KISCKASGYSFTGYTMNWVKQSHGKNLEWMGLINPYKGVSTYNQKFKDKA TLTVDKSSSTAYMELLSLTSEDSAVYYCARSGYYGDSDWYFDVWGQGTTL TVFSGGGGSGGGGSGGGGSFPKIPIEELEDRVFVNCNTSITWVEGTVGTL LSDITRLDLGKRILDPRGIYRCNGTDIYKDKESTVQVHYRMCQSCVELDP ATVAGIIVTDVIATLLLALGVFCFAGHETGRLSGAADTQALLRNDQVYQP LRDRDDAQYSHLGGNWARNK

SEQ ID NO: 46 (sdAbBCMA-anti-CD3 scFv-se; BCMA sdAb is underlined; anti-CD3 scFv is bolded; se is italicized; signal peptide sequence is bolded and italicized; CD8 hinge is squared)

MQSGTHWRVLGLCLLSVGVWGQ EVQLVESGGGLVQAGGSLRLSCAASGRT FTMGWFRQAPGKEREFVAAISLSPTLAYYAESVKGRFTISRDNAKNTVVL QMNSLKPEDTALYYCAADRKSVMSIRPDYWGQGTQVTVSSGGGGSGGGGS
GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSTTTPAPRPPTPAPTIASQPLSLRPEACRPAAGGAVHTRGL
DFACDVMSVATIVIVDICITGGLLLLVYYWSKNRKAKAKPVTRGAGAGGR QRGQNKERPPPVPNPDYEPIRKGQRDLYSGLNQRRI

SEQ ID NO: 47 (sdAbBCMA-anti-CD3 scFv-sg; BCMA sdAb is underlined; anti-CD3 scFv is bolded; sg is italicized; signal peptide sequence is bolded and italicized; CD8 hinge is squared)

MEQGKGLAVLILAIILLQGTLA EVQLVESGGGLVQAGGSLRLSCAASGRT FTMGWFRQAPGKEREFVAAISLSPTLAYYAESVKGRFTISRDNAKNTVVL QMNSLKPEDTALYYCAADRKSVMSIRPDYWGQGTQVTVSSGGGGSGGGGS
GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSTTTPAPRPPTPAPTIASQPLSLRPEACRPAAGGAVHTRGL
DFACDGFLFAEIVSIFVLAVGVYFIAGQDGVRQSRASDKQTLLPNDQLYQ PLKDREDDQYSHLQGNQLRRN

SEQ ID NO: 48 (sdAbBCMA-anti-CD3 scFv-sd; BCMA sdAb is underlined; anti-CD3 scFv is bolded; sd is italicized; signal peptide sequence is bolded and italicized; CD8 hinge is squared)

MQSGTHWRVLGLCLLSVGVWGQ EVQLVESGGGLVQAGGSLRLSCAASGRT FTMGWFRQAPGKEREFVAAISLSPTLAYYAESVKGRFTISRDNAKNTVVL QMNSLKPEDTALYYCAADRKSVMSIRPDYWGQGTQVTVSSGGGGSGGGGS
GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSTTTPAPRPPTPAPTIASQPLSLRPEACRPAAGGAVHTRGL
DFACDGIIVTDVIATLLLALGVFCFAGHETGRLSGAADTQALLRNDQVYQ PLRDRDDAQYSHLGGNWARNK

SEQ ID NO: 49 (sdAbBCMA-anti-CD3 scFv-taC/tbC; P2A sequence is underlined; signal peptide sequence is bolded; sdAbBCMA-anti-CD3 scFv-taC sequence is italicized; sdAbBCMA-anti-CD3 scFv-tbC sequence is squared)

MAMLLGASVLILWLQPDWVNSEVQLVESGGGLVQAGGSLRLSCAASGRTF TMGWFRQAPGKEREFVAAISLSPTLAYYAESVKGRFTISRDNAKNTVVLQ MNSLKPEDTALYYCAADRKSVMSIRPDYWGQGTQVTVSSGGGGGSGGGGS GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK 4TLTVDKSSSTAYMELLSLTSEDSAVYYCARSGYYGDSDWYFDVWGQGTT LTVFSGGGGSGGGGSGGGGSPNIQNPDPAVYQLRDSKSSDKSVCLFTDFD SQTNVSQSKDSDVYITDKTVLDMRSMDFKSNSAVAWSNKSDFACANAFNN SIIPEDTFFPSPESSCDVKLVEKSFETDTNLNFQNLSVIGFRILLLKVAG FNLLMTLRLWSSGSGATNFSLLKQAGDVEENPGPMGTSLLCWMALCLLGA DHADTEVQLVESGGGLVQAGGSLRLSCAASGRTFTMGWFRQAPGKEREFV
AAISLSPTLAYYAESVKGRFTISRDNAKNTVVLQMNSLKPEDTALYYCAA
DRKSVMSIRPDYWGQGTQVTVSSGGGGGSGGGGSGGGGSMDIQMTQTTSS
LSASLGDRVTISCRASQDIRNYLNWYQQKPDGTVKLLIYYTSRLHSGVPS
KFSGSGSGTDYSLTISNLEQEDIATYFCQQGNTLPWTFAGGTKLEIKGGG
GSGGGGSGGGGSGGGGSEVQLQQSGPELVKPGASMKISCKASGYSFTGYT
MNWVKQSHGKNLEWMGLINPYKGVSTYNQKFKDKATLTVDKSSSTAYMEL
LSLTSEDSAVYYCARSGYYGDSDWYFDVWGQGTLTVFSGGGGSGGGGSGG
GGSEDLNKVFPPEVAVFEPSEAEISHTQKATLVCLATGFFPDHVELSWWV
NGKEVHSGVSTDPQPLKEQPALNDSRYCLSSRLRVSATFWQNPRNHFRCQ
VQFYGLSENDEWTQDRAKPVTQIVSAEAWGRADCGFTSVSYQQGVLSATI
LYEILLGKATLYAVLVSALVLMAMVKRKDF

SEQ ID NO: 50 (sdAbBCMA-anti-CD3 scFv-tgC/tdC; P2A sequence is underlined; signal peptide sequence is bolded; sdAbBCMA-anti-CD3 scFv-tgC sequence is italicized; sdAbBCMA-anti-CD3 scFv-tdC sequence is squared)

MRWALLVLLAFLSPASQEVQLVESGGGLVQAGGSLRLSCAASGRTFTMGW FRQAPGKEREFVAAISLSPTLAYYAESVKGRFTISRDNAKNTVVLQMNSL KPEDTALYYCAADRKSVMSIRPDYWGQGTQVTVSSGGGGSGGGGSGGGGS GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSGGGGSGGGGSDKQLDADVSPKPTIFLPSIAETKLQKAGTY LCLLEKFFPDVIKIHWQEKKSNTILGSQEGNTMKTNDTYMKFSWLTVPEK SLDKEHRCIVRHENNKNGVDQEIIFPPIKTDVITMDPKDNCSKDANDTLL LQLTNTSAYYMYLLLLLKSWYFAlITCCLLRRTAFCCNGEKSGSGATNFS LLKQAGDVEENPGPMILTVGFSFLFFYRGTLCEVQLVESGGGLVQAGGSL
RLSCAASGRTFTMGWFRQAPGKEREFVAAISLSPTLAYYAESVKGRFTIS
RDNAKNTVVLQMNSLKPEDTALYYCAADRKSVMSIRPDYWGQGTQVTVSS
GGGGSGGGGSGGGGSGGGGSMDIQMTQTTSSLSASLGDRVTISCRASQDI
RNYLNWYQQKPDGTVKLLIYYTSRLHSGVPSKFSGSGSGTDYSLTISNLE
QEDIATYFCQQGNTLPWTFAGGTKLEIKGGGGSGGGGSGGGGSGGGGSEV
QLQQSGPELVKPGASMKISCKASGYSFTYTMNWVKQSHGKNLEWMGLINP
YKGVSTYNQKFKDKATLTVDKSSSTAYMELLSLTSEDSAVYYCARSGYYG
DSDWYFDVWGQGTTLTVFSGGGGSGGGGSGGGGSSQPHTKPSVFVMKNGT
NVACLVKEFYPKDIRINLVSSKKITEFDPAIVISPSGKYNAVKLGKYEDS
NSVTCSVQHDNKTVHSTDFEVKTDSTDHVKPKETENTKQPSKSCHKPKAI
VHTEKVNMMSLTVLGLRMLFAKTVAVNFLLTAKLFFL

SEQ ID NO: 51 (sdAbBCMA-anti-CD3 scFv-taC; BCMA sdAb is underlined; anti-CD3 scFv is bolded; taC is italicized; signal peptide sequence is bolded and italicized; linker is squared)

MAMLLGASVLILWLQPDWVNS EVQLVESGGGLVQAGGSLRLSCAASGRTF TMGWFRQAPGKEREFVAAISLSPTLAYYAESVKGRFTISRDNAKNTVVLO MNSLKPEDTALYYCAADRKSVMSIRPDYWGQGTQVTVSSGGGGGSGGGGS
GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSGGGGSGGGGSPNIQNPDPAVYQLRDSKSSDKSVCLFTDFD SQTNVSQSKDSDVYITDKTVLDMRSMDFKSNSAVAWSNKSDFACANAFNN SIIPEDTFFPSPESSCDVKLVEKSFETDTNLNFQNLSVIGFRILLLKVAG FNLLMTLRLWSS

SEQ ID NO: 52 (sdAbBCMA-anti-CD3 scFv-tbC; BCMA sdAb is underlined; anti-CD3 scFv is bolded; tbC is italicized; signal peptide sequence is bolded and italicized; linker is squared)

MGTSLLCWMALCLLGADHADT EVQLVESGGGLVQAGGSLRLSCAASGRTF TMGWFRQAPGKEREFVAAISLSPTLAYYAESVKGRFTISRDNAKNTVVLQ MNSLKPEDTALYYCAADRKSVMSIRPDYWGQGTQVTVSSGGGGGSGGGGS
GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSGGGGSGGGGSEDLNKVFPPEVAVFEPSEAEISHTQKATLV CLATGFFPDHVELSWWUNGKEVHSGVSTDPQPLKEQPALNDSRYCLSSRL RVSATFWQNPRNHFRCQVQFYGLSENDEWTQDRAKPVTQIVSAEAWGRAD CGFTSVSYQQGVLSATILYEILLGKATLYAVLVSALVLMAMVKRKDF

SEQ ID NO: 53 (sdAbBCMA-anti-CD3 scFv-tgC; BCMA sdAb is underlined; anti-CD3 scFv is bolded; tgC is italicized; signal peptide sequence is bolded and italicized; linker is squared)

MAMLLGASVLILWLQPDWVNS EVQLVESGGGLVQAGGSLRLSCAASGRTF TMGWFRQAPGKEREFVAAISLSPTLAYYAESVKGRFTISRDNAKNTVVLQ MNSLKPEDTALYYCAADRKSVMSIRPDYWGQGTQVTVSSGGGGGSGGGGS
GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSGGGGSGGGGSPMCWPDPAVYQLRDSKSSDKSVCLFTDFDS QTNVSQSKDSDVYITDKTVLDMRSMDFKSNSAVAWSNKSDFACANAFNNS IIPEDTFFPSPESSCDVKLVEKSFETDTNLNFQNLSVIGFRILLLKVAGF NLLIMTLRLWSS

SEQ ID NO: 54 (sdAbBCMA-anti-CD3 scFv-tdC; BCMA sdAb is underlined; anti-CD3 scFv is bolded; tdC is italicized; signal peptide sequence is bolded and italicized; linker is squared)

MGTSLLCWMALCLLGADHADT EVQLVESGGGLVQAGGSLRLSCAASGRTF TMGWFRQAPGKEREFVAAISLSPTLAYYAESVKGRFTISRDNAKNTVVLQ MNSLKPEDTALYYCAADRKSVMSIRPDYWGQGTQVTVSSGGGGGSGGGGS
GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSGGGGSGGGGSEDLNKVFPPEVAVFEPSEAEISHTQKATLV CLATGFFPDHVELSWWVNGKEVHSGVSTDPQPLKEQPALNDSRYCLSSRL RVSATFWQNPRNHFRCQVQFYGLSENDEWTQDRAKPVTQIVSAEAWGRAD CGFTSVSYQQGVLSATILYEILLGKATLYAVLVSALVLMAMVKRKDF

SEQ ID NO: 55 (sdAbBCMA-anti-CD3 scFv-sta; BCMA sdAb is underlined; anti-CD3 scFv is bolded; sta is italicized; signal peptide sequence is bolded and italicized; linker is squared)

MAMLLGASVLILWLQPDWVNS EVQLVESGGGLVQAGGSLRLSCAASGRTF TMGWFRQAPGKEREFVAAISLSPTLAYYAESVKGRFTISRDNAKNTVVLQ MNSLKPEDTALYYCAADRKSVMSIRPDYWGQGTQVTVSSGGGGGSGGGGS
GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSGGGGSGGGGSVIGFRILLLKVAGFNLLMTLRLWSS

SEQ ID NO: 56 (sdAbBCMA-anti-CD3 scFv-stb; BCMA sdAb is underlined; anti-CD3 scFv is bolded; stb is italicized; signal peptide sequence is bolded and italicized; linker is squared)

MAMLLGASVLILWLQPDWVNS EVQLVESGGGLVQAGGSLRLSCAASGRTF TMGWFRQAPGKEREFVAAISLSPTLAYYAESVKGRFTISRDNAKNTVVLO MNSLKPEDTALYYCAADRKSVMSIRPDYWGQGTQVTVSSGGGGGSGGGGS
GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSGGGGSGGGGSILLGKATLYAVLVSALVLMAMVKRKDF

SEQ ID NO: 57 (sdAbBCMA-anti-CD3 scFv-stg; BCMA sdAb is underlined; anti-CD3 scFv is bolded; stg is italicized; signal peptide sequence is bolded and italicized; linker is squared)

MAMLLGASVLILWLQPDWVNS EVQLVESGGGLVQAGGSLRLSCAASGRTF TMGWFRQAPGKEREFVAAISLSPTLAYYAESVKGRFTISRDNAKNTVVLO MNSLKPEDTALYYCAADRKSVMSIRPDYWGQGTQVTVSSGGGGGSGGGGS
GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSGGGGSGGGGSYYMYLLLLLKSVVYFAIITCCLLRRTAFCC NGEKS

SEQ ID NO: 58 (sdAbBCMA-anti-CD3 scFv-std; BCMA sdAb is underlined; anti-CD3 scFv is bolded; std is italicized; signal peptide sequence is bolded and italicized; linker is squared)

MAMLLGASVLILWLQPDWVNS EVQLVESGGGLVQAGGSLRLSCAASGRTF TMGWFRQAPGKEREFVAAISLSPTLAYYAESVKGRFTISRDNAKNTVVLO MNSLKPEDTALYYCAADRKSVMSIRPDYWGQGTQVTVSSGGGGGSGGGGS
GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSGGGGSGGGGSLGLRMLFAKTVAVNFLLTAKLFFL

SEQ ID NO: 59 (sdAbBCMA-anti-CD3 scFv-sta/stb; P2A sequence is underlined; signal peptide sequence is bolded; sdAbBCMA-anti-CD3 scFv-sta sequence is italicized; sdAbBCMA-anti-CD3 scFv-stb sequence is squared)

MAMLLGASVLILWLQPDWVNSEVQLVESGGGLVQAGGSLRLSCAASGRTF TMGWFRQAPGKEREFVAAISLSPTLAYYAESVKGRFTISRDNAKNTVVLQ MNSLKPEDTALYYCAADRKSVMSIRPDYWGQGTQVTVSSGGGGGSGGGGS GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSGGGGSGGGGSVIGFRILLLKVAGFNLLMTLRLWSSGSGAT NFSLLKQAGDVEENPGPMAMLLGASVLILWLQPDWVNSEVQLVESGGGLV
QAGGSLRLSCAASGRTFTMGWFRQAPGKEREFVAAISLSPTLAYYAESVK
GRFTISRDNAKNTVVLQMNSLKPEDTALYYCAADRKSVMSIRPDYWGQGT
QVTVSSGGGGGSGGGGSGGGGSMDIQMTQTTSSLSASLGDRVTISCRASQ
DIRNYLNWYQQKPDGTVKLLIYYTSRLHSGVPSKFSGSGSGTDYSLTISN
LEQEDIATYFCQQGNTLPWTFAGGTKLEIKGGGGSGGGGSGGGGSGGGGS
EVQLQQSGPELVKPGASMKISCKASGYSFTGYTMNWVKQSHGKNLEWMGL
INPYKGVSTYNQKFKDKATLTVDKSSSTAYMELLSLTSEDSAVYYCARSG
YYGDSDWYFDVWGQGTTLTVFSGGGGSGGGGSGGGGSILLGKATLYAVLV
SALVLMAMVKRKDF

SEQ ID NO: 60 (sdAbBCMA-anti-CD3 scFv-stg/std; P2A sequence is underlined; signal peptide sequence is bolded; sdAbBCMA-anti-CD3 scFv-stg sequence is italicized; sdAbBCMA-anti-CD3 scFv-std sequence is squared)

MAMLLGASVLILWLQPDWVNSEVQLVESGGGLVQAGGSLRLSCAASGRTF TMGWFRQAPGKEREFVAAISLSPTLAYYAESVKGRFTISRDNAKNTVVLQ MNSLKPEDTALYYCAADRKSVMSIRPDYWGQGTQVTVSSGGGGGSGGGGS GGGGSMDIQMTQTTSSLSASLGDRVTISCRASQDIRNYLNWYQQKPDGTV KLLIYYTSRLHSGVPSKFSGSGSGTDYSLTISNLEQEDIATYFCQQGNTL PWTFAGGTKLEIKGGGGSGGGGSGGGGSGGGGSEVQLQQSGPELVKPGAS MKISCKASGYSFTGYTMNWVKQSHGKNLEWMGLINPYKGVSTYNQKFKDK ATLTVDKSSSTAYMELLSLTSEDSAVYYCARSGYYGDSDWYFDVWGQGTT LTVFSGGGGSGGGGSGGGGSYYMYLLLLLKSVVYFAIITCCLLRRTAFCC NGEKSGSGATNFSLLKAGDVEENPGPMAMLLGASVLILWLQPDWVNSEVQ
LVESGGGLVQAGGSLRLSCAASGRTFTMGWFQAPGKEREFVAAISLSPTL
AYYAESVKGRFTISRDNAKNTVVLQMNSLKPEDTALYYCAADRKSVMSIR
PDYWGQGTQVTVSSGGGGGSGGGGSGGGGSMDIQMTQTTSSLSASLGDRV
TISCRASQDIRNYLNWYQQKPDGTVKLLIYYTSRLHSGVPSKFSGSGSGT
DYSLTISNLEQEDIATYFCQQGNTLPWTFAGGTKLEIKGGGGSGGGGSGG
GGSGGGGSEVQLQQSGPELVKPGASMKISCKASGYSFTGYTMNWVKQSHG
KNLEWMGLINPYKGVSTYNQKFKDKATLTVDKSSSTAYMELLSLTSEDSA
VYYCARSGYYGDSDWYFDVWGQGTTLTVFSGGGGSGGGGSGGGGSLGLRM
LFAKTVAVNFLLTAKLFFL

SEQ ID NO: 61 (tandem sdAbBCMA-anti-CD3 scFv-e; BCMA sdAb is underlined; anti-CD3 scFv is bolded; CD3_{ε} is italicized; signal peptide sequence is bolded and italicized; linker is squared)

MQSGTHWRVLGLCLLSVGVWGQ EVQLVESGGGLVQAGGSLRLSCAASGRTF TMGWFRQAPGKEREFVAAISLSPTLAYYAESVKGRFTISRDNAKNTVVLQ MNSLKPEDTALYYCAADRKSVMSIRPDYWGQGTQVTVSSGGGGSEVQLVE SGGGLVQAGGSLRLSCAASGRTFTMGWFRQAPGKEREFVAAISLSPTLAY YAESVKGRFTISRDNAKNTVVLQMNSLKPEDTALYYCAADRKSVMSIRPD YWGQGTQVTVSSGGGSGGGGSGGGGSMDIQMTQTTSSLSASLGDRVTISC RASQDIRNYLNWYQQKPDGTVKLLIYYTSRLHSGVPSKFSGSGSGTDYSL TISNLEQEDIATYFCQQGNTLPWTFAGGTKLEIKGGGGSGGGGSGGGGSG GGGSEVQLQQSGPELVKPGASMKISCKASGYSFTGYTMNWVKQSHGKNLE WMGLINPYKGVSTYNQKFKDKATLTVDKSSSTAYMELLSLTSEDSAVYYC ARSGYYGDSDWYFDVWGQGTTLTVFSGGGGSGGGGSGGGGSDGNEEMGGI TQTPYKVSISGTTVILTCPQYPGSEILWQHNDKNIGGDEDDKNIGSDEDH LSLKEFSELEQSGYYVCYPRGSKPEDANFYLYLRARVCENCMEMDVMSVA TIVIVDICITGGLLLLVYYWSKNRKAKAKPVTRGAGAGGRQRGQNKERPP PVPNPDYEPIRKGQRDLYSGLNQRRI

SEQ ID NO: 62 (sdAbBCMA-anti-TCR Ab-tgC; BCMA sdAb is underlined; anti-TCR Ab is bolded; tgC is italicized; signal peptide sequence is bolded and italicized; linker is squared)

MRWALLVLLAFLSPASQ EVQLVESGGGLVQAGGSLRLSCAASGRTFTMGW FRQAPGKEREFVAAISLSPTLAYYAESVKGRFTISRDNAKNTVVLQMNSL KPEDTALYYCAADRKSVMSIRPDYWGQGTQVTVSSGGGGSGGGGSGGGGS
GGGGSQVQLQQSGPELVKPGASVKISCKISDYTFSNSWMNWVKQRPGQGL EWIGRIYPGDGDTKYNGEFKAKATLTADKSARTAYMQLNNLTSVDSAVYF CARWERYDGGFTYWGQGTLVTVSPGGGGSGGGGSGGGGSDIQMTQSPASL SASVGETVTITCRASENIYSYLSWYQQKQGKSPQLLVYYANTLAEGVPSR FSGSGYGTQFSLKINSLQPVDFGNYYCQHHYGLPPTFGGGTKLEIKGGGG
SGGGGSGGGGSDKQLDADVSPKPTIFLPSIAETKLQKAGTYLCLLEKFFP DVIKIHWQEKKSNTILGSQEGNTMKTNDTYMKFSWLTVPEKSLDKEHRCI VRHENNKNGVDQEIIFPPIKTDVITMDPKDNCSKDANDTLLLQLTNTSAY YMYLLLLLKSWYFAIITCCLLRRTAFCCNGEKS

SEQ ID NO: 63 (sdAbBCMA-anti-TCR Ab-tdC; BCMA sdAb is underlined; anti-TCR Ab is bolded; tdC is italicized; signal peptide sequence is bolded and italicized; linker is squared)

MILTVGFSFLFFYRGTL CEVQLVESGGGLVQAGGSLRLSCAASGRTFTMG WFRQAPGKEREFVAAISLSPTLAYYAESVKGRFTISRDNAKNTVVLQMNS LKPEDTALYYCAADRKSVMSIRPDYWGQGTQVTVSSGGGGSGGGGSGGGG
SGGGGSQVQLQQSGPELVKPGASVKISCKISDYTFSNSWMNWVKQRPGQG LEWIGRIYPGDGDTKYNGEFKAKATLTADKSARTAYMQLNNLTSVDSAVY FCARWERYDGGFTYWGQGTLVTVSPGGGGSGGGGSGGGGSDIQMTQSPAS LSASVGETVTITCRASENIYSYLSWYQQKQGKSPQLLVYYANTLAEGVPS RFSGSGYGTQFSLKINSLQPVDFGNYYCQHHYGLPPTFGGGTKLEIKGGG
GSGGGGSGGGGSQPHTKPSVFVMKNGTNVACLVKEFYPKDIRINLVSSKK ITEFDPAIVISPSGKYNAVKLGKYEDSNSVTCSVQHDNKTVHSTDFEVKT DSTDHVKPKETENTKQPSKSCHKPKAIVHTEKVNMMSLTVLGLRMLFAKT VAVNFLLTAKLFFL

SEQ ID NO: 64 (sdAbBCMA-anti-TCR Ab-tgC/tdC; P2A sequence is underlined; signal peptide sequence is bolded; sdAbBCMA-anti-TCR Ab-tgC sequence is italicized; sdAbBCMA-anti-TCR Ab-tdC sequence is squared)

MRWALLVLLAFLSPASQEVQLVESGGGLVQAGGSLRLSCAASGRTFTMG WFRQAPGKEREFVAAISLSPTLAYYAESVKGRFTISRDNAKNTVVLQMNS LKPEDTALYYCAADRKSVMSIRPDYWGQGTQVTVSSGGGGSGGGGSGGGG SGGGGSQVQLQQSGPELVKPGASVKISCKISDYTFSNSWMNWVKQRPGQG LEWIGRIYPGDGDTKYNGEFKAKATLTADKSARTAYMQLNNLTSVDSAVY FCARWERYDGGFTYWGQGTLVTVSPGGGGSGGGGSGGGGSDIQMTQSPAS LSASVGETVTITCRASENIYSYLSWYQQKQGKSPQLLVYYANTLAEGVPS RFSGSGYGTQFSLKINSLQPVDFGNYYCQHHYGLPPTFGGGTKLEIKGGG GSGGGGSGGGGSDKQLDADVSPKPTIFLPSIAETKLQKAGTYLCLLEKFF PDVIKIHWQEKKSNTILGSQEGNTMKTNDTYMKFSWLTVPEKSLDKEHRC IVRHENNKNGVDQEIIFPPIKTDVITMDPKDNCSKDANDTLLLQLTNTSA YYMYLLLLLKSVVYFAIITCCLLRRTAFCCNGEKSGSGATNFSLLKQAGD VEENPGPMILTVGFSFLFFYRGTLCEVQLVESGGGLVQAGGSLRLSCAAS
GRTFTMGWFRQAPGKEREFVAAISLSPTLAYYAESVKGRFTISRDNAKNT
VVLQMNSLKPEDTALYYCAADRKSVMSIRPDYWGQGTQVTVSSGGGGSGG
GGSGGGGSGGGGSQVQLQQSGPELVKPGASVKISCKISDYTFSNSWMNWV
IKQRPGQGLEWIGRIYPGDGDTKYNGEFKAKATLTADKSARTAYMQLNNL
TSVDSAVYFCARWERYDGGFTYWGQGTLVTVSPGGGGSGGGGSGGGGSDI
QMTQSPASLSASVGETVTITCRASENIYSYLSWYQQKQGKSPQLLVYYAN
TLAEGVPSRFSGSGYGTQFSLKINSLQPVDFGNYYCQHHYGLPPTFGGGT
KLEIKGGGGSGGGGSGGGGSSQPHTKPSVFVMKNGTNVACLVKEFYPKDI
RINLVSSKKITEFDPAIVISPSGKYNAVKLGKYEDSNSVTCSVQHDNKTV
HSTDFEVKTDSTDHVKPKETENTKQPSKSCHKPKAIVHTEKVNMMSLTVL
GLRMLFAKTVAVNFLLTAKLFFL

SEQ ID NO: 65 (sdAbBCMA-anti-CD3 scFv-CD4; BCMA sdAb is underlined; anti-CD3 scFv is bolded; CD4 transmembrane domain and intracellular domain is italicized; signal peptide sequence is bolded and italicized; linker is squared)

MDFQVQIFSFLLISASVI EVQLVESGGGLVQAGGSLRLSCAASGRTFTMG WFRQAPGKEREFVAAISLSPTLAYYAESVKGRFTISRDNAKNTVVLQMNS LKPEDTALYYCAADRKSVMSIRPDYWGQGTQVTVSSGGGGSGGGGSGGGG
SMDIQMTQTTSSLSASLGDRVTISCRASQDIRNYLNWYQQKPDGTVKLLI YYTSRLHSGVPSKFSGSGSGTDYSLTISNLEQEDIATYFCQQGNTLPWTF AGGTKLEIKGGGGSGGGGSGGGGSGGGGSEVQLQQSGPELVKPGASMKIS CKASGYSFTGYTMNWVKQSHGKNLEWMGLINPYKGVSTYNQKFKDKATLT VDKSSSTAYMELLSLTSEDSAVYYCARSGYYGDSDWYFDVWGQGTTLTVF SGGGGSSGQVLLESNIKVLPTWSTPVQPMALIVLGGVAGLLLFIGLGIFF CVRCRHRRRQAERMSQIKRLLSEKKTCQCPHRFQKTCSPI

SEQ ID NO: 66 (P2A amino acid sequence)

GSGATNFSLLKQAGDVEENPGP

SEQ ID NO: 67 (linker)

GGGGGSGGGGSGGGGS

SEQ ID NO: 68 (linker)

GGGGSS

1. A chimeric receptor polypeptide comprising:
a) an extracellular target binding domain;
b) an extracellular T-cell receptor (TCR) binding domain;
c) a transmembrane domain comprising a transmembrane domain of a first TCR subunit; and
d) an intracellular domain comprising an intracellular domain of a second TCR subunit,
wherein the first TCR subunit and the second TCR subunit are each independently selected from the group consisting of CD3ε, CD3γ, CD3δ, TCRα, TCRβ, TCRγ, and TCRδ.

2. The chimeric receptor polypeptide of claim 1, wherein the extracellular TCR binding domain comprises a TCR antigen binding domain specifically recognizing a TCR subunit selected from the group consisting of TCRα, TCRβ, TCRγ, TCRδ, CD3ε, CD3δ, CD3γ, and CD3ζ.

3. (canceled)

4. The chimeric receptor polypeptide of claim 2, wherein the TCR antigen binding domain is a single chain Fv (scFv) or a single domain antibody (sdAb).

5. The chimeric receptor polypeptide of claim 1, wherein the extracellular TCR binding domain comprises two or more TCR antigen binding domains arranged in tandem.

6. The chimeric receptor polypeptide of claim 1, wherein the chimeric receptor polypeptide does not comprise an extracellular domain of the first TCR subunit, the second TCR subunit, or any TCR subunit.

7. (canceled)

8. The chimeric receptor polypeptide of claim 1, wherein the chimeric receptor polypeptide does not comprise an intracellular co-stimulatory domain.

9. The chimeric receptor polypeptide of claim 1, wherein the first TCR subunit and the second TCR subunit are different.

10-11. (canceled)

12. The chimeric receptor polypeptide of claim 1, wherein the first TCR subunit and the second TCR subunit are the same.

13-19. (canceled)

20. The chimeric receptor polypeptide of claim 1, wherein the extracellular target binding domain is at the N-terminus of the extracellular TCR binding domain.

21. The chimeric receptor polypeptide of claim 1, wherein the extracellular target binding domain is at the C-terminus of the extracellular TCR binding domain.

22. The chimeric receptor polypeptide of claim 1, wherein the extracellular target binding domain comprises a target antigen binding domain specifically recognizing a target antigen.

23. The chimeric receptor polypeptide of claim 22, wherein the target antigen binding domain is an scFv, an sdAb, or a designed ankyrin repeat protein (DARPin).

24. The chimeric receptor polypeptide of claim 22, wherein the extracellular target binding domain comprises two or more target antigen binding domains arranged in tandem.

25-26. (canceled)

27. The chimeric receptor polypeptide of claim 22, wherein the target antigen is selected from the group consisting of BCMA, NY-ESO-1, VEGFR2, MAGE-A3, AFP, CD4, CD19, CD20, CD22, CD30, CD33, CD38, CD70, CD123, CEA, EGFR (such as EGFRvIII), GD2, GPC-2, GPC3, HER2, LILRB4, IL-13Rα2, IGF1R, mesothelin, PSMA, ROR1, WT1, NKG2D, CLL1, TGFaRII, TGFbRII, CCR5, CXCR4, CCR4, HPV related antigen, and EBV related antigen (such as LMP1 and LMP2).

28-31. (canceled)

32. The chimeric receptor polypeptide of claim 1, comprising from the N-terminus to the C-terminus:
a) optional signal peptide - extracellular target binding domain - optional first linker -extracellular TCR binding domain - optional second linker - optional hinge region -transmembrane domain - intracellular domain; or
b) optional signal peptide - extracellular TCR binding domain - optional first linker -extracellular target binding domain - optional second linker - optional hinge region -transmembrane domain - intracellular domain.

33. An isolated nucleic acid encoding the chimeric receptor polypeptide of claim 1.

34. A nucleic acid vector comprising one or more nucleic acids of claim 33.

35. An isolated immune cell comprising one or more chimeric receptor polypeptides of claim 1.

36-41. (canceled)

42. A pharmaceutical composition comprising the isolated immune cell of claim 35, and an optional pharmaceutically acceptable excipient.

43. A method of treating a disease in an individual, comprising administering to the individual an effective amount of the pharmaceutical composition of claim 42.

44-46. (canceled)


SEQUENCE LISTING (68 sequences)

SEQ ID NO: 1 - protein, 5 aa, Artificial Sequence
  linker
GGGGS

SEQ ID NO: 2 - protein, 10 aa, Artificial Sequence
  linker
GGGGSGGGGS

SEQ ID NO: 3 - protein, 15 aa, Artificial Sequence
  linker
GGGGSGGGGSGGGGS

SEQ ID NO: 4 - protein, 20 aa, Artificial Sequence
  linker
GGGGSGGGGSGGGGSGGGGS

SEQ ID NO: 5 - protein, 6 aa, Artificial Sequence
  linker
GGSGGS

SEQ ID NO: 6 - protein, 6 aa, Artificial Sequence
  linker
SGGGGS

SEQ ID NO: 7 - protein, 12 aa, Artificial Sequence
  linker
GRAGGGGAGGGG

SEQ ID NO: 8 - protein, 6 aa, Artificial Sequence
  linker
GRAGGG

SEQ ID NO: 9 - protein, 1 aa, Artificial Sequence
  linker
  VARIANT (1)...(1): Can be present in repeats of one or more
G

SEQ ID NO: 10 - protein, 2 aa, Artificial Sequence
  linker
  VARIANT (1)...(2): Can be present in repeats of one or more
GS

SEQ ID NO: 11 - protein, 5 aa, Artificial Sequence
  linker
  VARIANT (1)...(5): Can be present in repeats of one or more
GSGGS

SEQ ID NO: 12 - protein, 4 aa, Artificial Sequence
  linker
  VARIANT (1)...(4): Can be present in repeats of one or more
GGGS

SEQ ID NO: 13 - protein, 5 aa, Artificial Sequence
  linker
  VARIANT (1)...(5): Can be present in repeats of one or more
GGGGS

SEQ ID NO: 14 - protein, 2 aa, Artificial Sequence
  linker
GG

SEQ ID NO: 15 - protein, 4 aa, Artificial Sequence
  linker
GGSG

SEQ ID NO: 16 - protein, 5 aa, Artificial Sequence
  linker
GGSGG

SEQ ID NO: 17 - protein, 5 aa, Artificial Sequence
  linker
GSGSG

SEQ ID NO: 18 - protein, 5 aa, Artificial Sequence
  linker
GSGGG

SEQ ID NO: 19 - protein, 5 aa, Artificial Sequence
  linker
GGGSG

SEQ ID NO: 20 - protein, 5 aa, Artificial Sequence
  linker
GSSSG

SEQ ID NO: 21 - protein, 229 aa, Artificial Sequence
  linker
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 117 aa, Artificial Sequence
  anti-CD3 sdAb
QVKLEESGGGLVQAGGSLRVSCTASGRTFDTMGWFRQAPGKEREFVAAVRWSSGNTLYGNTVKGRFTISRDTATNTVYLQMSSLKHEDTAVYYCAARVGGRGAADHWGQGTQVTVSS

SEQ ID NO: 23 - protein, 122 aa, Artificial Sequence
  anti-CD3 sdAb
QVQLVESGGGLVQAGGSLRLSCAASGRTFSSRAVGWFRQAPGKERQFVAAIDSGGGETGYADSVKGRFAISRDNAKNTVYLQMNSLKPEDTAVYYCAVADLLVTWPRAYKYWGQGTQVTVSS

SEQ ID NO: 24 - protein, 250 aa, Artificial Sequence
  anti-CD3 scFv (UCHT1)
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 25 - protein, 6 aa, Artificial Sequence
  MMP seq
PLGLAG

SEQ ID NO: 26 - protein, 118 aa, Artificial Sequence
  anti-BCMA sdAb
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 27 - protein, 241 aa, Artificial Sequence
  anti-TCR gamma/delta Monoclonal Antibody (B1.1)
QVQLQQSGPELVKPGASVKISCKISDYTFSNSWMNWVKQRPGQGLEWIGRIYPGDGDTKYNGEFKAKATLTADKSARTAYMQLNNLTSVDSAVYFCARWERYDGGFTYWGQGTLVTVSPGGGGSGGGGSGGGGSDIQMTQSPASLSASVGETVTITCRASENIYSYLSWYQQKQGKSPQLLVYYANTLAEGVPSRFSGSGYGTQFSLKINSLQPVDFGNYYCQHHYGLPPTFGGGTKLEIK

SEQ ID NO: 28 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 29 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 30 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 31 - protein, 45 aa, Artificial Sequence
  CD8 hinge
TTTPAPRPPTPAPTIASQPLSLRPEACRPAAGGAVHTRGLDFACD

SEQ ID NO: 32 - protein, 142 aa, Artificial Sequence
  taC
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 33 - protein, 177 aa, Artificial Sequence
  tbC
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 34 - protein, 25 aa, Artificial Sequence
  sta
VIGFRILLLKVAGFNLLMTLRLWSS

SEQ ID NO: 35 - protein, 27 aa, Artificial Sequence
  stb
ILLGKATLYAVLVSALVLMAMVKRKDF

SEQ ID NO: 36 - protein, 173 aa, Artificial Sequence
  tgC
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 37 - protein, 153 aa, Artificial Sequence
  tdC
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 38 - protein, 35 aa, Artificial Sequence
  stg
YYMYLLLLLKSVVYFAIITCCLLRRTAFCCNGEKS

SEQ ID NO: 39 - protein, 24 aa, Artificial Sequence
  std
LGLRMLFAKTVAVNFLLTAKLFFL

SEQ ID NO: 40 - protein, 81 aa, Artificial Sequence
  se (CD3 Epsilon no extracellular)
VMSVATIVIVDICITGGLLLLVYYWSKNRKAKAKPVTRGAGAGGRQRGQNKERPPPVPNPDYEPIRKGQRDLYSGLNQRRI

SEQ ID NO: 41 - protein, 66 aa, Artificial Sequence
  sg (CD3 Gamma no extracellular)
GFLFAEIVSIFVLAVGVYFIAGQDGVRQSRASDKQTLLPNDQLYQPLKDREDDQYSHLQGNQLRRN

SEQ ID NO: 42 - protein, 66 aa, Artificial Sequence
  sd (CD3 Delta no extracellular)
GIIVTDVIATLLLALGVFCFAGHETGRLSGAADTQALLRNDQVYQPLRDRDDAQYSHLGGNWARNK

SEQ ID NO: 43 - protein, 605 aa, Artificial Sequence
  sdAbBCMA-anti-CD3 scFv-e
MQSGTHWRVLGLCLLSVGVWGQEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 44 - protein, 580 aa, Artificial Sequence
  sdAbBCMA-anti-CD3 scFv-g
MEQGKGLAVLILAIILLQGTLAEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 45 - protein, 570 aa, Artificial Sequence
  sdAbBCMA-anti-CD3 scFv-d
MQSGTHWRVLGLCLLSVGVWGQEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 46 - protein, 536 aa, Artificial Sequence
  sdAbBCMA-anti-CD3 scFv-se
MQSGTHWRVLGLCLLSVGVWGQEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSTTTPAPRPPTPAPTIASQPLSLRPEACRPAAGGAVHTRGLDFACDVMSVATIVIVDICITGGLLLLVYYWSKNRKAKAKPVTRGAGAGGRQRGQNKERPPPVPNPDYEPIRKGQRDLYSGLNQRRI

SEQ ID NO: 47 - protein, 521 aa, Artificial Sequence
  sdAbBCMA-anti-CD3 scFv-sg
MEQGKGLAVLILAIILLQGTLAEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSTTTPAPRPPTPAPTIASQPLSLRPEACRPAAGGAVHTRGLDFACDGFLFAEIVSIFVLAVGVYFIAGQDGVRQSRASDKQTLLPNDQLYQPLKDREDDQYSHLQGNQLRRN

SEQ ID NO: 48 - protein, 521 aa, Artificial Sequence
  sdAbBCMA-anti-CD3 scFv-sd
MQSGTHWRVLGLCLLSVGVWGQEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSTTTPAPRPPTPAPTIASQPLSLRPEACRPAAGGAVHTRGLDFACDGIIVTDVIATLLLALGVFCFAGHETGRLSGAADTQALLRNDQVYQPLRDRDDAQYSHLGGNWARNK

SEQ ID NO: 49 - protein, 1181 aa, Artificial Sequence
  sdAbBCMA-anti-CD3 scFv-taC/tbC
MAMLLGASVLILWLQPDWVNSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMGTSLLCWMALCLLGADHADTEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 50 - protein, 1189 aa, Artificial Sequence
  sdAbBCMA-anti-CD3 scFv-tgC/tdC
MRWALLVLLAFLSPASQEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGATNFSLLKQAGDVEENPGPMILTVGFSFLFFYRGTLCEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 51 - protein, 562 aa, Artificial Sequence
  sdAbBCMA-anti-CD3 scFv-taC
MAMLLGASVLILWLQPDWVNSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 52 - protein, 597 aa, Artificial Sequence
  sdAbBCMA-anti-CD3 scFv-tbC
MGTSLLCWMALCLLGADHADTEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 53 - protein, 562 aa, Artificial Sequence; identical to SEQ ID NO: 51
  sdAbBCMA-anti-CD3 scFv-tgC

SEQ ID NO: 54 - protein, 597 aa, Artificial Sequence; identical to SEQ ID NO: 52
  sdAbBCMA-anti-CD3 scFv-tdC

SEQ ID NO: 55 - protein, 445 aa, Artificial Sequence
  sdAbBCMA-anti-CD3 scFv-sta
MAMLLGASVLILWLQPDWVNSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSVIGFRILLLKVAGFNLLMTLRLWSS

SEQ ID NO: 56 - protein, 447 aa, Artificial Sequence
  sdAbBCMA-anti-CD3 scFv-stb
MAMLLGASVLILWLQPDWVNSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSILLGKATLYAVLVSALVLMAMVKRKDF

SEQ ID NO: 57 - protein, 455 aa, Artificial Sequence
  sdAbBCMA-anti-CD3 scFv-stg
MAMLLGASVLILWLQPDWVNSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSYYMYLLLLLKSVVYFAIITCCLLRRTAFCCNGEKS

SEQ ID NO: 58 - protein, 444 aa, Artificial Sequence
  sdAbBCMA-anti-CD3 scFv-std
MAMLLGASVLILWLQPDWVNSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSLGLRMLFAKTVAVNFLLTAKLFFL

SEQ ID NO: 59 - protein, 914 aa, Artificial Sequence
  sdAbBCMA-anti-CD3 scFv-sta/stb
MAMLLGASVLILWLQPDWVNSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSVIGFRILLLKVAGFNLLMTLRLWSSGSGATNFSLLKQAGDVEENPGPMAMLLGASVLILWLQPDWVNSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSILLGKATLYAVLVSALVLMAMVKRKDF

SEQ ID NO: 60 - protein, 921 aa, Artificial Sequence
  sdAbBCMA-anti-CD3 scFv-stg/std
MAMLLGASVLILWLQPDWVNSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSYYMYLLLLLKSVVYFAIITCCLLRRTAFCCNGEKSGSGATNFSLLKQAGDVEENPGPMAMLLGASVLILWLQPDWVNSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSLGLRMLFAKTVAVNFLLTAKLFFL

SEQ ID NO: 61 - protein, 728 aa, Artificial Sequence
  tandem sdAbBCMA-anti-CD3 scFv-e
MQSGTHWRVLGLCLLSVGVWGQEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 62 - protein, 584 aa, Artificial Sequence
  sdAbBCMA-anti-TCR Ab-tgC
MRWALLVLLAFLSPASQEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSGGGGSQVQLQQSGPELVKPGASVKISCKISDYTFSNSWMNWVKQRPGQGLEWIGRIYPGDGDTKYNGEFKAKATLTADKSARTAYMQLNNLTSVDSAVYFCARWERYDGGFTYWGQGTLVTVSPGGGGSGGGGSGGGGSDIQMTQSPASLSASVGETVTITCRASENIYSYLSWYQQKQGKSPQLLVYYANTLAEGVPSRFSGSGYGTQFSLKINSLQPVDFGNYYCQHHYGLPPTFGGGTKLEIKGGGGSGGGGSGGGGSDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 63 - protein, 565 aa, Artificial Sequence
  sdAbBCMA-anti-TCR Ab-tdC
MILTVGFSFLFFYRGTLCEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSGGGGSQVQLQQSGPELVKPGASVKISCKISDYTFSNSWMNWVKQRPGQGLEWIGRIYPGDGDTKYNGEFKAKATLTADKSARTAYMQLNNLTSVDSAVYFCARWERYDGGFTYWGQGTLVTVSPGGGGSGGGGSGGGGSDIQMTQSPASLSASVGETVTITCRASENIYSYLSWYQQKQGKSPQLLVYYANTLAEGVPSRFSGSGYGTQFSLKINSLQPVDFGNYYCQHHYGLPPTFGGGTKLEIKGGGGSGGGGSGGGGSSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 64 - protein, 1171 aa, Artificial Sequence
  sdAbBCMA-anti-TCR Ab-tgC/tdC
MRWALLVLLAFLSPASQEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSGGGGSQVQLQQSGPELVKPGASVKISCKISDYTFSNSWMNWVKQRPGQGLEWIGRIYPGDGDTKYNGEFKAKATLTADKSARTAYMQLNNLTSVDSAVYFCARWERYDGGFTYWGQGTLVTVSPGGGGSGGGGSGGGGSDIQMTQSPASLSASVGETVTITCRASENIYSYLSWYQQKQGKSPQLLVYYANTLAEGVPSRFSGSGYGTQFSLKINSLQPVDFGNYYCQHHYGLPPTFGGGTKLEIKGGGGSGGGGSGGGGSDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGATNFSLLKQAGDVEENPGPMILTVGFSFLFFYRGTLCEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSGGGGSQVQLQQSGPELVKPGASVKISCKISDYTFSNSWMNWVKQRPGQGLEWIGRIYPGDGDTKYNGEFKAKATLTADKSARTAYMQLNNLTSVDSAVYFCARWERYDGGFTYWGQGTLVTVSPGGGGSGGGGSGGGGSDIQMTQSPASLSASVGETVTITCRASENIYSYLSWYQQKQGKSPQLLVYYANTLAEGVPSRFSGSGYGTQFSLKINSLQPVDFGNYYCQHHYGLPPTFGGGTKLEIKGGGGSGGGGSGGGGSSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 65 - protein, 490 aa, Artificial Sequence
  sdAbBCMA-anti-CD3 scFv-CD4
MDFQVQIFSFLLISASVIEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSGGGGSGGGGSGGGGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 66 - protein, 22 aa, Artificial Sequence
  P2A
GSGATNFSLLKQAGDVEENPGP

SEQ ID NO: 67 - protein, 16 aa, Artificial Sequence
  linker
GGGGGSGGGGSGGGGS

SEQ ID NO: 68 - protein, 6 aa, Artificial Sequence
  linker
GGGGSS